(19) United States
(12) Patent Application Publication
(10) Pub. No.: US 2025/0134504 A1
(43) Pub. Date: May 1, 2025

(54) Systems for Atherectomy and Pulsatile Intravascular Lithotripsy and Methods for Using the Same

(71) Applicant: Amplitude Vascular Systems, Inc., Boston, MA (US)

(72) Inventors: Robert Chisena, Boston, MA (US); Sean Gilligan, Ponte Vedra Beach, FL (US)

(21) Appl. No.: 18/691,387

(22) PCT Filed: Feb. 6, 2024
(86) PCT No.: PCT/US2024/014611

§ 371 Date: Mar. 12, 2024

Related U.S. Application Data
(60) Provisional application No. 63/444,414, filed on Feb. 9, 2023.

(51) Int. Cl. A61B 17/00; A61B 17/22; A61B 17/3207; A61B 90/00

(52) U.S. Cl. CPC A61B 17/00234; A61B 17/22012; A61B 17/320758; A61B 90/06; A61B 2017/00075; A61B 2017/00084; A61B 2017/00154; A61B 2017/00238; A61B 2017/00305; A61B 2017/00464; A61B 2017/00477; A61B 2017/00778; A61B 2017/22025; A61B 2017/22038; A61B 2017/22051; A61B 2017/320766; A61B 2090/066

(57) ABSTRACT

Systems configured for performing atherectomy and pulsatile intravascular lithotripsy are provided. Aspects of the systems include a console; and a handle, wherein the handle is configured to be interchangeably operably connectable to: an atherectomy subsystem, and a pulsatile intravascular lithotripsy subsystem. Also provided are components of the systems that include the same. The systems and assemblies find use in a variety of different applications, including combined atherectomy and pulsatile intravascular lithotripsy procedures as well as balloon angioplasty applications.

### CROSS-REFERENCE TO RELATED APPLICATIONS

Pursuant to 35 U.S.C. § 119 (e), this application claims priority to the filing date of U.S. provisional patent application Ser. No. 63/444,414 filed Feb. 9, 2023, the disclosure of which application is incorporated herein by reference in its entirety.

### INTRODUCTION

Ischemic heart disease, the number one cause of death in the world, is caused by atherosclerotic plaque build-up within human vasculature. Worldwide, these diseases represent 84.5% of cardiovascular deaths and 28.2% of overall mortality. Ischemic heart disease is developed through a mechanism called atherosclerosis, which is the accumulation of fatty and calcified materials that cause stenosis, the narrowing of the arterial lumen. Both the coronary and peripheral arteries may suffer from atherosclerotic plaque accumulation. The plaque buildup from atherosclerosis limits blood flow through these arteries and can lead to major adverse cardiovascular events such as myocardial infarction, limb amputation, and mortality. In the early stages of atherosclerosis, plaques are soft and fatty, but as time passes and the disease progresses, these plaques physically harden, or calcify. Calcified plaque (CP) that develops in the innermost layer of the artery wall occurs most frequently. CP results from the deposition and remodeling of calcium hydroxyapatite, a process that mimics bone formation. Vessels with CP have reduced vascular elasticity and impaired vessel perfusion. Because of this reduced compliance and perfusion, the presence of CP in the vasculature is associated with an increased risk of mortality and other adverse events.

While many patients with CP are asymptomatic, a substantial number of patients develop symptoms and signs related to ischemia and undergo endovascular or surgical repair. Endovascular approaches are generally favored over surgical treatments because of the less invasive nature of the approach. However, often the strength of the calcified plaque may pose special challenges for effective intravascular treatments. There are many common intravascular treatments used to treat CP. One such method is balloon angioplasty (BA), in which the CP lesion is pre-dilated. During BA, a balloon is advanced to the affected lesion and is expanded to dilate a plaque-burdened vessel to restore normal blood flow. If successful, the pre-dilation step is followed by secondary therapies such as applying drug-coated balloons or stents. For successful pre-dilation, BA must mechanically fracture the CP to ensure the long-term opening, or patency, of the vessel and to re-establish the elasticity of the surrounding healthy vessel. Often, high-pressure, non-compliant balloons are used to achieve success. However, because of the strength of CP, full balloon expansion is often restricted, and the CP remains unfractured.

Another treatment strategy directed to fracturing CP is cutting and scoring balloon angioplasty. Cutting balloons, which are balloons surrounded by sharp-tipped metallic blades, and scoring balloons, which are balloons constrained in a metallic cage, aim to generate stress concentrations for CP fracture. During balloon pressurization, the metallic blades or cage can become embedded in soft-tissue or CP causing major procedural issues. Poor outcomes have been associated with these balloons including restenosis in 20-30% of cases and major adverse events such as vessel perforation, myocardial infarction, or death in 6% of cases.

Shockwave intravascular therapy employs low-pressure balloons with embedded shockwave-generating lithotripters. Short-term efficacy and safety with lithotripsy devices have been demonstrated through clinical trials; however, recent case reports have shown that these >50 ATM cavitation explosions may lead to dangerous arterial dissections and perforations.

Another commonly employed treatment for CP is atherectomy, a technique that uses grinding, for example, to modulate or debulk CP. However, atherectomy is more challenging technically and may grind surrounding healthy tissue in addition to CP, which can lead to long-term vessel injury.

Other treatment methods combine standard treatment approaches to overcome the difficulty in the strength of the CP. One such procedure that clinicians have developed is referred to as Rota-Shock (Mclaughlin et al., “First United States Experience with Rota-Shock: A Case Series”, Cardiovascular Revascularization Medicine (2022) 40:209-213), which is a combination of Boston Scientific's Rotablator™ rotational atherectomy system with Shockwave Medical's intravascular lithotripsy platform. Using the Rotablator™ rotational atherectomy system, a small grinding burr is first used to create a channel in the narrow or completely closed occlusion. Once the channel is created, a Shockwave Medical intravascular lithotripsy balloon is passed through the channel and across the lesion. Intravascular lithotripsy energy is delivered to the surrounding tissue, which creates radial fractures in the calcium and allows the vessel to expand. While the Rota-Shock approach may provide benefits in certain situations, this method is bulky, time consuming, and requires the insertion and removal of multiple mechanical systems into vasculature. Aspects of the present invention address these drawbacks.

### SUMMARY

Systems for performing atherectomy and pulsatile intravascular lithotripsy are provided. Aspects of the systems include a console; and a handle, wherein the handle is configured to be interchangeably operably connectable to: an atherectomy subsystem, and a pulsatile intravascular lithotripsy subsystem. In some embodiments, the handle is operably connected to the atherectomy subsystem. In other embodiments, the handle is operably connected to the pulsatile intravascular lithotripsy subsystem. In embodiments, the atherectomy subsystem and the pulsatile intravascular lithotripsy subsystem each comprises an interface configured to operably connect to an interface of the handle. In some embodiments, the atherectomy subsystem comprises: an atherectomy tool that is a rotational atherectomy tool or an orbital atherectomy tool or an orbital atherectomy tool or a laser tool or an ultrasound tool or an electrohydraulic lithotripsy (EHL) cavitation emitter tool or a mechanotransduction tool.

Embodiments of systems of the invention further comprise a rotational assembly configured to transduce energy transmitted from the console into rotational energy. In some embodiments, the atherectomy subsystem comprises: a lateral transmission assembly configured to propagate rotational energy from the rotational assembly to an atherectomy tool. In other embodiments, the atherectomy subsystem comprises: a sensor configured to sense one or more of: current, rotational position, speed, acceleration, temperature, linear position, torque, pressure or flow.

Embodiments of systems of the invention comprise a pulsatile intravascular lithotripsy subsystem comprising: a proximal connector configured to operably connect to the handle, a distal balloon, and a catheter, wherein the distal balloon is operably connected to the catheter, and the catheter is operably connected to the proximal connector. In certain such embodiments, the proximal connector and a connector of the atherectomy subsystem each comprise an identical handle interface.

Embodiments of systems of the invention further comprise an integrated atherectomy and pulsatile intravascular lithotripsy subsystem comprising: the atherectomy subsystem, and the pulsatile intravascular lithotripsy subsystem. In some embodiments, the pulsatile intravascular lithotripsy subsystem comprises a guidewire lumen, the atherectomy subsystem comprises a lateral transmission assembly comprising a guidewire, and the guidewire is present within the guidewire lumen. In certain such cases, the atherectomy tool is present on a distal region of the guidewire.

Methods for treating a diseased vessel. Methods according to embodiment of the invention comprise deploying a system according to an embodiment of the present invention so that an atherectomy tool of the system is adjacent to an occlusion of a diseased vessel, actuating the system such that the atherectomy tool creates a channel in the occlusion of the diseased vessel, guiding a distal balloon of the system through the channel, and actuating the system to impart pulsatile energy to the diseased vessel. Methods according to other embodiments of the invention comprise introducing a guidewire into luminal tissue; using the guidewire to introduce into luminal tissue an atherectomy tool of an atherectomy subsystem of a system according to an embodiment of the present invention; removing the atherectomy subsystem from the luminal tissue; and using the guidewire to introduce into luminal tissue a distal balloon of a pulsatile intravascular lithotripsy subsystem of the system.

### BRIEF DESCRIPTION OF THE FIGURES

FIG. 1A provides a schematic diagram of aspects of a system in accordance with embodiments of the invention. FIG. 1B provides a schematic diagram of aspects of an atherectomy subsystem of a system in accordance with embodiments of the invention. FIG. 1C provides a schematic diagram of aspects of a pulsatile intravascular lithotripsy subsystem of a system in accordance with embodiments of the invention.

FIG. 2A provides a schematic diagram of aspects of another system in accordance with embodiments of the invention with combined tools. FIG. 2B provides a schematic diagram of aspects of an atherectomy subsystem and a pulsatile intravascular subsystem with combined tools in accordance with embodiments of the invention.

FIG. 3A provides a view of a console according to embodiments of the invention. FIG. 3B provides a view of another console according to other embodiments of the invention.

FIG. 4A provides a view of a handle according to an embodiment of the invention; FIG. 4B provides a view of a handle according to another embodiment of the invention as well as a view of a connector according to an embodiment of the invention, illustrating the releasable and operable interconnection between such elements.

FIG. 5A is a schematic view of an elastic conduit (e.g., artery) having a hardened material (e.g., calcified plaque) embedded therein to be treated by the dynamic balloon angioplasty (DBA) techniques and devices according to some embodiments of the present teachings. FIG. 5B is a schematic view of the elastic conduit of FIG. 5A having a DBA angioplasty balloon navigated to the affected region and pre-pressurized. FIG. 5C is a schematic view of the elastic conduit of FIG. 5A having the DBA angioplasty balloon cycled to a low pressure. FIG. 5D is a schematic view of the elastic conduit of FIG. 5A having the DBA angioplasty balloon cycled to a high pressure. FIG. 5E is a schematic view of the elastic conduit of FIG. 5A having the hardened material fractured according to the principles of the present teachings.

FIG. 6 provides a depiction of a pulsatile treatment plan in accordance with an embodiment of the invention.

### DETAILED DESCRIPTION

Disclosed herein are systems and methods for combining two procedures, atherectomy and pulsatile intravascular lithotripsy, into a single treatment modality. In embodiments, systems of the invention include a console; and a handle, wherein the handle is configured to be interchangeably operably connectable to: an atherectomy subsystem, and a pulsatile intravascular lithotripsy subsystem. In some embodiments, the handle is operably connected to the atherectomy subsystem. In other embodiments, the handle is operably connected to the pulsatile intravascular lithotripsy subsystem. Aspects of the invention further include methods of performing atherectomy followed by pulsatile intravascular lithotripsy to treat CP. Methods of the invention comprise operably connecting a handle to an atherectomy subsystem and engaging the atherectomy subsystem to perform an atherectomy procedure, and operably connecting the handle to pulsatile intravascular lithotripsy subsystem and engaging the pulsatile intravascular lithotripsy subsystem to perform a pulsatile intravascular lithotripsy procedure. In further describing various aspects of the invention, the systems and components thereof are described first in greater detail, followed by a review of methods of using the systems.

As described, systems of the invention include a console; and a handle, wherein the handle is configured to be interchangeably operably connectable to: an atherectomy subsystem, and a pulsatile intravascular lithotripsy subsystem. Further, in certain embodiments, the handle is operably connected to the atherectomy subsystem, and in other embodiments, the handle is operably connected to the pulsatile intravascular lithotripsy subsystem. By “interchangeably operably connectable,” it is meant, for example, that embodiments of handles of the invention comprise a single interface that is configured such that the same handle interface can be connected to either an atherectomy subsystem or a pulsatile intravascular lithotripsy subsystem. That is, in embodiments, “interchangeably operably connectable,” means that a handle may first be operably connected to an atherectomy subsystem such that the system may be used to perform an atherectomy procedure, after which procedure is performed, the handle may be disengaged from such subsystem and then operably connected to the pulsatile intravascular lithotripsy subsystem such that the system may be used to perform a pulsatile intravascular lithotripsy procedure. In embodiments, the handle interface may comprise one or more structural elements, such as a shape or depth or other volumetric features, connector elements, such as a pneumatic connector, including, for example, an O-ring and/or seating for an O-ring, alignment elements, keying elements, electrical connections, or the like, where the arrangement and shape of such elements of the interface are such that they align with corresponding elements of each of the atherectomy subsystem and pulsatile intravascular lithotripsy subsystem. That is, in embodiments, each of the atherectomy subsystem and pulsatile intravascular subsystem comprise an interface with alignment or other connection features, such as those described above and herein, that correspond with analogous features of the handle interface. The interfaces of each of the handle and the subsystems are arranged or otherwise configured such that, when the handle is connected to either subsystem, potential energy may be transmitted from the handle to the connected subsystem, as described in detail herein.

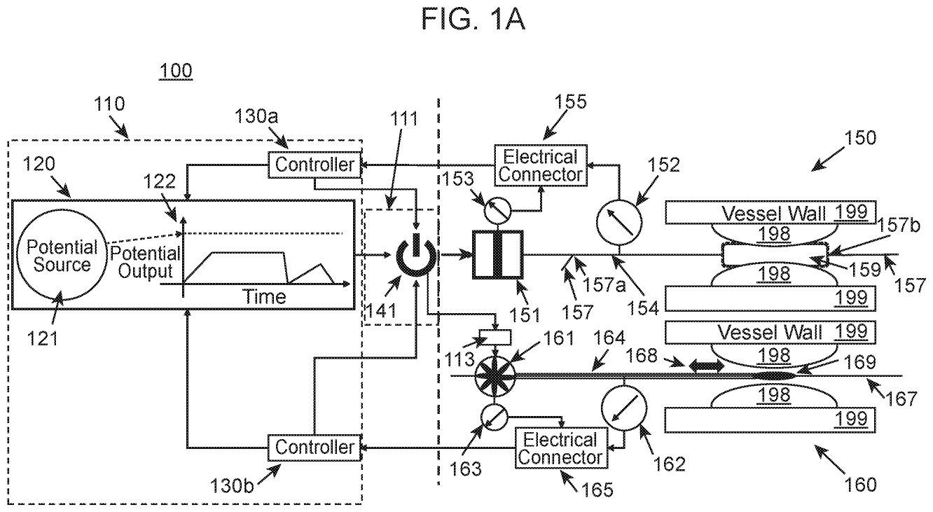

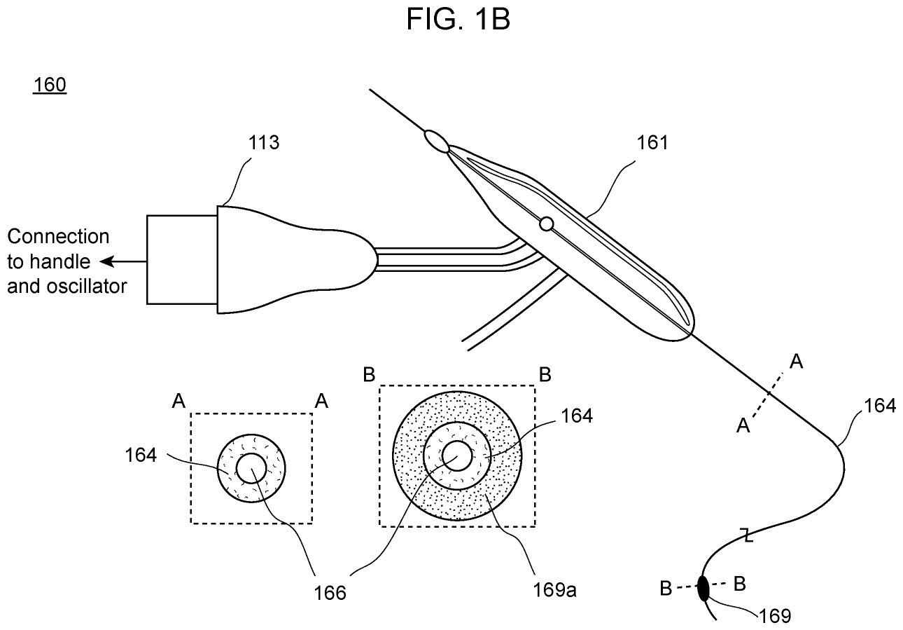

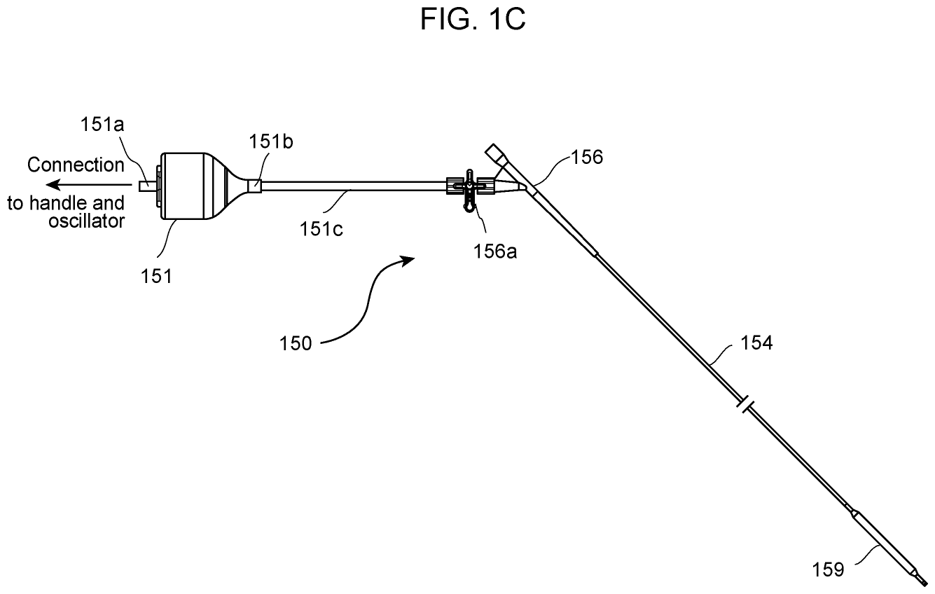

FIGS. 1A-C depict system 100, illustrating a combined atherectomy and pulsatile intravascular lithotripsy platform with separate tools. System 100 may also be referred to as a combined atherectomy and pulsatile intravascular lithotripsy platform with non-integrated tools. System 100 comprises separate tools in part, because distal balloon 159 is separate from atherectomy tool 169. That is, distal balloon 159 and atherectomy tool 169 are not directly connected to one another, e.g., via catheter 154 or lateral transmission assembly 164 or guidewire 167 or guidewire 157 or another mechanism, when such tools are present within vessel wall 199. Lateral transmission assembly may also be referred to as lateral transmission element.

As described herein, atherectomy and pulsatile intravascular lithotripsy subsystems 160 and 150 are configured such that each subsystem is interchangeable operably connectable to handle 111. That is, the system may be configured such that the atherectomy subsystem 160 may be first connected to handle 111 and subsequently, pulsatile intravascular lithotripsy subsystem 150 may be connected to handle 111. Handle 111 and atherectomy and pulsatile intravascular lithotripsy subsystems 160 and 150 are configured such that the handle and each subsystem can be operably connected, subsequently released and subsequently operably connected. Handle 111 comprises an interface, e.g., a shape and/or other interlocking elements, such as keyed surfaces or alignment features and electrical connectors, positioned such that when handle 111 interfaces with atherectomy subsystem 160, handle 111 supplies potential energy to atherectomy subsystem 160, as well as any control or data interconnections, such as electrical connectors, for supplying electrical potential to power circuitry, e.g., controllers, or sending/receiving sensor data or the like. Similarly, a shape and/or any other interlocking elements of handle 111 are positioned such that handle 111 also interfaces with pulsatile intravascular lithotripsy subsystem 150 such that handle 111 can supply potential energy to pulsatile intravascular lithotripsy subsystem 150, as well as any control or data interconnections or the like. In embodiments, handle interfaces with connector 113 of atherectomy subsystem 160 and proximal connector 151 of pulsatile intravascular lithotripsy subsystem 150, such that the same handle 111 (as well as the same console 110) may be used to supply potential energy to each of the tools of atherectomy subsystem 160 and pulsatile intravascular lithotripsy subsystem 150. Connectors 113 and 151 may interchangeably be referred to as amplifiers or amplifier assemblies.

FIGS. 1A-C provide a schematic of each of the atherectomy and pulsatile intravascular lithotripsy subsystems 160 and 150, respectively, each connecting to common handle 111 and console 110 in accordance with an embodiment of the invention. As illustrated in FIG. 1A, the system 100 includes a console 110 with a potential source 121 that is set to provide an output 122 of potential energy, which output may vary, such as a pre-determined output, user-set output, or feedback/feedforward-controlled output. Console 110 may also be referred to as a console assembly or a console subsystem.

The potential source 121 can vary, where examples of potential sources include, but are not limited to, electromagnetic, such as electrical voltage or current, potential sources or a high-pressure gas, such as nitrogen, carbon dioxide, compressed air or mixtures of gases, etc. The potential source output 122 can be set to a certain potential output 122 such as voltage or pressure or another output such as current or flow rate. This output 122 can be adjusted, e.g., via a regulator (as described herein), between a minimum and maximum level (e.g., minimum and maximum level or pressure or voltage or current, etc.), which may or may not exceed the level of the input potential source 121.

In some cases, multiple potential sources may be used at once or at separate times. For example, multiple potential sources may be used via a single console or multiple consoles and/or a single handle or multiple handles. As illustrated, the potential output 122 is transmitted to handle 111 comprising switch 141, for example an electronic and or mechanical switch, solenoid or the like. Switch 141 is located within handle 111. Handle 111 may comprise multiple output connections such as an output to a pulsatile intravascular lithotripsy subsystem 150 with distal balloon 159 and/or an output to an atherectomy subsystem 160 with atherectomy tool 169; or handle 111 may comprise a single output connection configured such that such single output connection can be operably connected to each of a pulsatile intravascular lithotripsy subsystem 150 and an atherectomy subsystem 160. That is, output of handle 111 comprising switch 141 may first be operably connected to atherectomy subsystem 160 and, upon disconnecting atherectomy subsystem 160, may subsequently be operably connected to pulsatile intravascular lithotripsy subsystem 150; i.e., handle 111 is configured (e.g., shaped and/or comprising interlocks) such that handle 111 may be interchangeable operably connected to each of atherectomy subsystem 160 and pulsatile intravascular lithotripsy subsystem 150.

Handle 111 may also be referred to as handle assembly. Handle 111 comprises switch 141 and may be operably connected to an amplifier assembly, e.g., amplifier assemblies 113, 151, corresponding to each of atherectomy subsystem 160 and pulsatile intravascular lithotripsy subsystem 150, respectively. Amplifier assemblies 113, 151 may also be referred to as amplifiers or proximal connectors or connectors. Amplifier assemblies 151, 113 and handle assembly 111 are each configured (e.g., shaped and/or comprising interlocks and/or a standardized shape and interconnections) such that handle assembly 111 can be releasably engaged (i.e. capable of being operably connected such that potential energy is transmitted from handle to amplifier assembly, and then disengaged) with each amplifier assembly 151, 113. In certain cases, handle assembly 111 is reusable (e.g., across different patients and/or different procedures), whereas the atherectomy subsystem 160 and pulsatile intravascular lithotripsy subsystem 150, including amplifier assemblies 151, 113, are disposable.

In embodiments, handle assembly 111 can be disengaged from, for example, amplifier assembly 113 in order to subsequently operably connect handle assembly 111 to, for example, amplifier assembly 151. For example, handle assembly 111 may first be operably connected with atherectomy amplifier assembly 113. That is, an operator of system 100 may first connect atherectomy subsystem 160 to handle 111 such that atherectomy subsystem 160 is operably connected to handle 111 and console 110 to perform an atherectomy procedure. The operator may then disengage handle assembly 111 from atherectomy amplifier assembly 113 and operably connect handle assembly 111 to pulsatile intravascular lithotripsy amplifier 151 such that pulsatile intravascular lithotripsy subsystem 150 is operably connected to handle 111 and console 110 to perform a pulsatile intravascular lithotripsy procedure. That is, while system 100 comprises separate atherectomy and pulsatile intravascular lithotripsy subsystems, such subsystems each connect to a common potential source (i.e., handle 111, which itself is operably connected to console 110) providing ease of use for an operator as well as time-savings for combined atherectomy and pulsatile intravascular lithotripsy procedures.

As described herein, embodiments of systems of the invention may further comprise a handle assembly 111, amplifier assembly 151 configured for a pulsatile intravascular lithotripsy subsystem 150, as well as amplifier assembly 113 configured for an atherectomy subsystem 160. In such embodiments, handle assembly 111 and amplifier assembly 113 can be disengaged from each other in order to subsequently operably connect the handle assembly 111 to another amplifier assembly, such as amplifier assembly 151. For example, a clinician may first insert atherectomy subsystem 160 into vessel 199, connect atherectomy amplifier 113 to handle 111, treat lesion 198, then disconnect atherectomy subsystem 160 from handle 11, then connect pulsatile intravascular lithotripsy amplifier 151 to handle 111 and treat lesion 198. Such steps may be repeated as needed during a combined atherectomy, pulsatile intravascular lithotripsy procedure.

Console 110 may be referred to as a console assembly or a console subsystem. In some embodiments as illustrated schematically in FIG. 1A, the system 100 includes a console 110, which may comprise one or more console units 120; a handle 111 comprising a switch 141; an atherectomy subsystem 160, which comprises connector 113, rotational assembly 161, lateral transmission assembly 164 and atherectomy tool 169; and a pulsatile intravascular lithotripsy subsystem 150, which comprises a proximal connector 151, as well as catheter 154 with a fluidic passage connecting distal and proximal regions of catheter 154 with distal balloon 159. Switch may be referred to as oscillator 141. In embodiments, switch 141 is located in handle 111. In embodiments, atherectomy subsystem 160 comprises rotational assembly 161 operably connected to connector 113, which connector 113 is configured to pass potential energy from handle 111 to rotational assembly 161.

Handle 111, comprising oscillator 141, is connected to atherectomy subsystem 160 for transmitting pulsatile energy (i.e., second pulse energy) or static energy to atherectomy tool 169 via connector 113, rotational assembly 161 and lateral transmission assembly 164. Atherectomy subsystem 160 is further configured to translate in distal and proximal directions, as shown by arrows 168. Handle 111, comprising oscillator 141, is connected to pulsatile intravascular lithotripsy subsystem 150 for transmitting pulsatile energy (i.e., second pulse energy) or static energy to distal balloon 159 via proximal connector 151 and catheter 154, for pressurizing balloon 159.

Console assembly 110 of system 100 comprises a single console 120. Console 120 may also be referred to as a console unit. However, embodiments of systems of the invention may comprise one or more console units. When a plurality of console units are employed, such console units may be combined into a single physical component (i.e., housing) or separated into a plurality of housings, e.g., one housing per each console unit. In certain such cases, console units are configured to be operated independently of each other, i.e., are capable of being controlled independently, whether the console units are present in a single housing or multiple housings. In some cases, a first console unit may be employed in connection with providing potential energy to atherectomy subsystem 160, and a second console unit may be employed in connection with providing potential energy to pulsatile intravascular lithotripsy subsystem 150.

Console unit 120 includes potential source 121 for generating or providing energy for transmission to handle 111 comprising switch 141 via a potential regulator 122 for modulating transmitted potential energy. In certain embodiments, potential source 121 may be separate from console unit 120 and console assembly 110. That is, potential source 121 may be operably connected to console assembly 110 but not enclosed within the same housing as console assembly 110. The output from potential source 121 may include regulated 122 or un-regulated potential energy, such as that from high-pressure fluid or voltage or current. A potential regulator 122 may be used to modify potential energy output from potential source 121 into a form that can be transmitted and further manipulated by switch 141. Switch 141 may also be referred to as an oscillator. Switch 141 can generate pulse energy for output to a subsystem from energy transmitted from potential source 121.

In some embodiments, multiple console units can be included in console assembly 110 and can operate substantially in parallel (i.e., independently) to generate multiple potential outputs 122 for transmission to multiple handles, each comprising an oscillator, or, in other cases, to a single handle with multiple oscillators present therein. In some embodiments, multiple console units 120 can be configured to generate multiple potential outputs 122 in cases where treatment of tissue, such as treatment of cardiovascular tissue, such as treatment of tissue with calcified plaque deposits, comprises applying different configurations of energy, such as pulsatile energy, to such tissue, for example, where the atherectomy subsystem and the pulsatile intravascular lithotripsy subsystem are configured to receive potential energy in different forms (e.g., electrical potential and high-pressure gas, respectively, etc.) and/or at different frequencies, duty cycles and/or amplitudes. In such instances, different potential outputs 122 may be separately applied to tissue, e.g., cardiovascular tissue, e.g., via the atherectomy subsystem or via the pulsatile intravascular lithotripsy subsystem, or at different times. That is, one potential source is operably connected to system 100 at a first time for use with an atherectomy procedure and, subsequently, another potential source is operably connected to system 100 at a second time for use with a pulsatile intravascular lithotripsy procedure. In other instances, potential outputs from multiple console units are combined, e.g., at a single handle. In still other instances, multiple console units are configured to generate multiple potential outputs including different forms of potential energy (e.g., high pressure fluid or voltage potential energy) in cases where treatment of tissue, such as cardiovascular tissue, such as treatment of tissue with calcified plaque deposits, requires application of different forms of energy. For example, an embodiment may comprise an atherectomy subsystem that utilizes a rotational tool as well as one or more of a laser tool or an ultrasound tool or an electrohydraulic lithotripsy (EHL) cavitation emitter tool or a mechanotransduction tool, etc.

Console assembly 110 further comprises controllers 130a, 130b, in each case, configured to receive input, such as, for example, control and/or data input signals, such as, sensor data signals, from at least one of console assembly 110, potential source 121, handle 111, switch 141, atherectomy subsystem 160, amplifier 113, pulsatile intravascular lithotripsy subsystem 150, proximal connector 151 or other aspects of system 100. In the depicted embodiment, controller 130a receives input from intravascular pulsatile lithotripsy subsystem 150. That is, controller 130a receives input signals originating from sensors 152, 153 of pulsatile intravascular lithotripsy subsystem 150, via electrical connector 155. Sensors 152, 153 may comprise any sensor configured to sense any relevant characteristic of pulsatile intravascular lithotripsy subsystem 150 capable of detection. For example, sensor 152 may comprise a pressure transducer configured to measure a pressure within pulsatile intravascular lithotripsy subsystem 150, such as, for example, a pressure of a fluid channel of catheter 154 or a pressure of an aspect of distal balloon 159 such as an angioplasty balloon, and sensor 153 may comprise a volume sensor (e.g., a displacement sensor, such as a Hall sensor, integrated into proximal connector 151, for example), configured to measure a volume of fluid present in or displaced into, for example, distal balloon 159. That is, where in such embodiment, operation of proximal connector 151 displaces fluid into catheter 154 and/or distal balloon 159.

Similarly, controller 130b receives input from atherectomy subsystem 160. That is, controller 130b receives input signals originating from sensors 162, 163 of atherectomy subsystem 160, via electrical connector 165. Sensors 162, 163 may comprise any convenient sensor configured to sense any relevant characteristic of atherectomy subsystem 160 capable of detection. For example, sensors 162, 163 may comprise sensors configured to sense rotational position, speed, acceleration, temperature, linear position, torque or pressure of aspects of lateral transmission assembly 164 or atherectomy tool 169 and/or may comprise a sensor configured to sense current associated with the atherectomy subsystem 160 interfacing with occluded lesion 198 or a current response to the atherectomy subsystem 160 penetrating occluded lesion 198; and/or sensors 162, 163 may be configured to sense one or more of rotational position, speed, acceleration, temperature, linear position, torque, pressure associated with rotational assembly 161 or atherectomy tool 169. In some cases, sensors 162, 163 may comprise an optical encoder or a rotational encoder or another mechanism configured for ascertaining the rotational motion or rotational displacement of aspects of the atherectomy subsystem, such as a position, velocity, acceleration or jerk.

In some cases, one or both of sensors 162, 163 are configured to measure torque, e.g., torque applied by atherectomy tool 169 to lesion 198. For example, such a torque sensor may be configured to sense how much resistance the atherectomy tool is encountering as it is engaged with lesion 198 or as it is advanced into lesion 198. Changes in torque measured by such sensor may be indicative of the extent to which atherectomy tool 169 has penetrated lesion 198. For example, a sudden drop in the amount of torque applied indicates that atherectomy tool 169 may no longer be grinding against aspects of lesion 198 (e.g., no longer griding against calcified plaque of lesion 198).

Input from sensors, such as sensors 162, 163, may be utilized in connection with controlling aspects of system 100, such as aspects of atherectomy system 160. For example, in embodiments, acceleration and/or rotational speed of atherectomy tool 169, as detected by one or more sensors, may be utilized in connection with establishing a control loop for controlling configurable aspects of the subsystem, such as, for example, the rotational speed of atherectomy tool or lateral motion (e.g., distal advancement) of atherectomy tool. Such control may take the form of a feedback or feedforward control technique. In some cases, a PID control loop mechanism is applied based on data detected by one or more sensors, such as sensors 162, 163. When rotational assembly 161 is operated based on electrical potential, a control strategy, such as the control loops described above, may be utilized in connection with controlling voltage or current applied to rotational assembly 161. In some cases, a sensor is present in system 100 that is configured to detect lateral motion, e.g., along arrows 168, of lateral transmission assembly 164. Any convenient positional sensor may be applied for such purpose, such as, for example, an optical encoder. In some cases, sensors of interest comprise sensors configured to detect fluid pressure, e.g., for sensing a pressure within system 100 or sensing pressure within vessel 199, for example.

In instances, controllers 130a, 130b may be configured to receive input from a plurality of sensors, including sensors configured to measure any relevant aspect of system 100 or the environment in which system 100 is applied (e.g., a pressure sensor, a temperature sensor, a volume sensor, a displacement sensor or the like) and may be configured to capture data from any location throughout system 100, including, for example, one or more locations of system 100, e.g., locations on atherectomy tool 169, lateral transmission assembly 164, connector 113, rotational assembly 161, distal balloon 159, catheter 154, proximal connector 151, handle 111, oscillator 141, console unit 120 or console assembly 110. Controllers 130a, 130b are located within console assembly 110. However, in other embodiments, controllers 130a, 130b may be present within handle 111 or within pulsatile intravascular lithotripsy subsystem 150 or proximal connector 151 or atherectomy subsystem 160 or amplifier 113 or rotational assembly 161, for example. Embodiments of systems may comprise one or more controllers, such as one, two, three, four, five or more controllers and such may be located within the same aspect or subsystem of the system or may be distributed in different locations throughout the system.

In general, in embodiments, sensors can be configured at any desired location of system 100 to gather any desired information regarding use of system 100, e.g., in connection with a treatment procedure. In other instances, controllers 130a, 130b are configured to receive input from user inputs such as buttons or switches for specifying treatment options, such as, for example, system pressures, frequencies, duty cycles, etc.

Controller 130a receives input from pressure transducer 152 of pulsatile intravascular lithotripsy subsystem 150 and, based at least in part on such input, generates a control signal for controlling aspects of console assembly 110, such as, for example, a magnitude of potential output 122, i.e., via an active regulator used to adjust a magnitude of potential output 122 (e.g., an output pressure). Similarly, controller 130b receives input from sensor 162 of atherectomy subsystem 160 and, based at least in part on such input, generates a control signal for controlling aspects of console assembly 110, such as, for example, a magnitude of potential output 122, i.e., via an active regulator used to adjust a magnitude of potential output 122 (e.g., an output pressure). In embodiments, controllers 130a, 130b may be combined into a single controller 130; i.e., such that controller 130 comprises logically distinct (but not physically distinct) controllers 130a, 130b.

An output of console assembly 110 is operably connected to handle 111 comprising oscillator 141, such that energy transmitted (i.e., regulated potential output 122) from potential source 121 of console unit 120 is transmitted to oscillator 141 of handle 111. Oscillator 141 is configured to generate pulsatile or static energy (e.g., energy of varying magnitudes over a time period or of a static magnitude over a time period) from energy transmitted from potential source 121 (i.e., regulated potential output 122). In instances, oscillator 141 may comprise a solenoid valve configured to either allow or interrupt transmission of energy to pulsatile intravascular lithotripsy subsystem 150 and/or atherectomy subsystem 160. In other instances, oscillator 141 may include any applicable electrical, e.g., electrical solenoid, optical or mechanical switch, as such are known in the art. As described herein, the behavior of oscillator 141 may be controlled by, for example, controllers 130a, 130b, based on any desired feedback, such as, for example, feedback from system 100 or external signals, such as, for example, inputs from an operator of system 100, such as, for example, a clinician.

Controllers 130a, 130b are shown connected to oscillator 141 within handle 111. In instances, oscillator 141 may be configured so that aspects of behavior of oscillator 141, such as, for example, an oscillation frequency and/or duty cycle, may be controlled by controllers 130a, 130b. For example, controllers 130a, 130b may control a position (e.g., an open or closed position) or other aspect, such as frequency or duty cycle, of the behavior of a solenoid of oscillator 141.

FIG. 1B provides a more detailed view of certain aspects of atherectomy subsystem 160 comprising amplifier 113 and rotational assembly 161, as seen in system 100 of FIG. 1A. Referring now to FIGS. 1A and 1B, atherectomy subsystem 160 is configured for removing or disrupting occlusions 198, such as, for example, calcified plaque deposits or chronic total occlusions (CTOs), in blood vessels 199, and includes lateral transmission assembly 164 having a distal end and a proximal end, the distal end being configured for insertion into vessel 199. In some cases, systems of the invention find use addressing calcified plaque deposits where the calcification prevents or inhibits compression of the plaque deposit. Atherectomy subsystem 160 further comprises atherectomy tool 169 that comprises a rotational cutting mechanism, located at the distal end of lateral transmission assembly 164, for cutting and removing occlusions 198 from blood vessel 199. Atherectomy tool 169 may comprise, for example, a burr. Atherectomy subsystem 160 further comprises amplifier 113 and rotational assembly 161, configured to be operably connected to handle 111 and potential source 121 of console 110. Connector 113 may also be referred to as an amplifier or as a proximal connector. Connector 113 is configured to physically connect to an output of handle 111 and transmit potential energy received therefrom to rotational assembly 161. In some cases, connector 113 is merely a pass-through device, configured to transmit potential energy received from handle 111 to rotational assembly 161. Rotational assembly 161 is configured to operably connect to the proximal end of lateral transmission assembly 164 for providing rotational power to atherectomy tool 169.

Atherectomy subsystem 160 comprises connector 113 that connects to handle 111. In embodiments, connector 113 may comprise a pass-through device. That is, connector 113 may be configured to allow potential energy, such as, for example, pressurized gas or electrical potential, etc., to pass through connector 113 to rotational assembly 113. In some cases, connector 113 is configured to allow pressurized gas to pass through connector 113 and into rotational assembly 161, such that pressurized gas is used to power rotation and/or orbit and/or lateral translation of atherectomy tool 169 via rotational assembly 161. In other cases, connector 113 is configured to allow electrical potential energy to pass through connector 113 and into rotational assembly 161, again to power movement, such as rotation and/or orbit and/or lateral translation, of atherectomy tool 169. In certain cases, connector 113 is used to provide an interface, e.g., a standard or uniform or modular interface, between rotational assembly 161 and output of handle 111. For example, handle 111 may be configured such that it comprises only a single output for transmitting potential energy to distal aspects of system 100, and such handle 111 output may be configured, e.g., comprising a shape and/or other interlocking features, such that it can operably connect to connector 161 as well as proximal connector 151 of pulsatile intravascular lithotripsy subsystem 150. In certain cases, the handle and connectors comprise interfaces that enable their use such that: connector 113 of atherectomy subsystem 160 is first operably connected to output of handle 111 and then subsequently disengaged from handle 111, and then subsequently proximal connector 151 of pulsatile intravascular lithotripsy subsystem 150 is operably connected to output of handle 111.

Embodiments of the present invention are advantageous relative to traditional approaches to atherectomy and/or lithotripsy at least insofar as each of the atherectomy subsystem 160 and pulsatile intravascular lithotripsy subsystem 150 may be operably connected to a single handle 111. That is, the use of a single handle 111 and console 120, to which each of atherectomy subsystem 160 and pulsatile intravascular lithotripsy subsystem 150 are separately operably connected simplifies and streamlines performing a combined atherectomy and pulsatile intravascular lithotripsy procedure.

Atherectomy subsystem 160 further comprises rotational assembly 161 operably connected, via laterally transmission element 164, to atherectomy tool 169, comprising, for example, a rotational cutting mechanism, and configured for controlling the rotational and/or orbital speed, direction, acceleration, etc. of the rotational cutting mechanism 169, as well as retrieving exhaust gas.

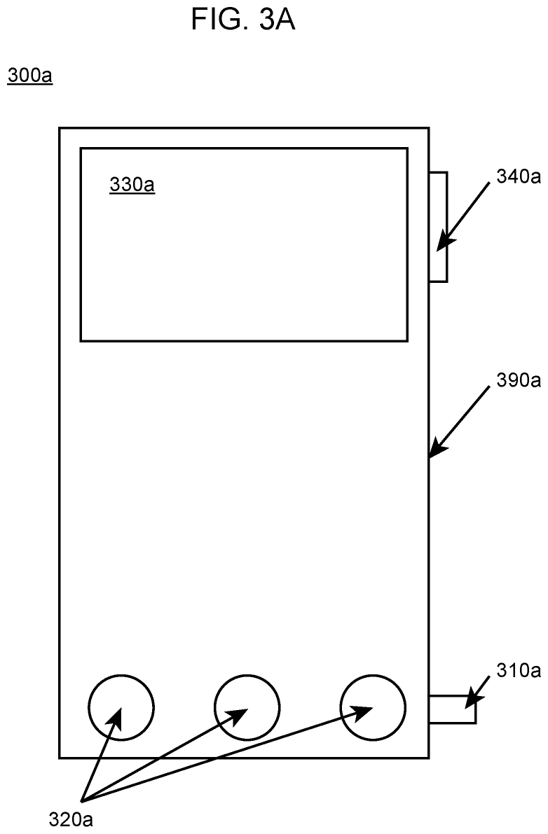

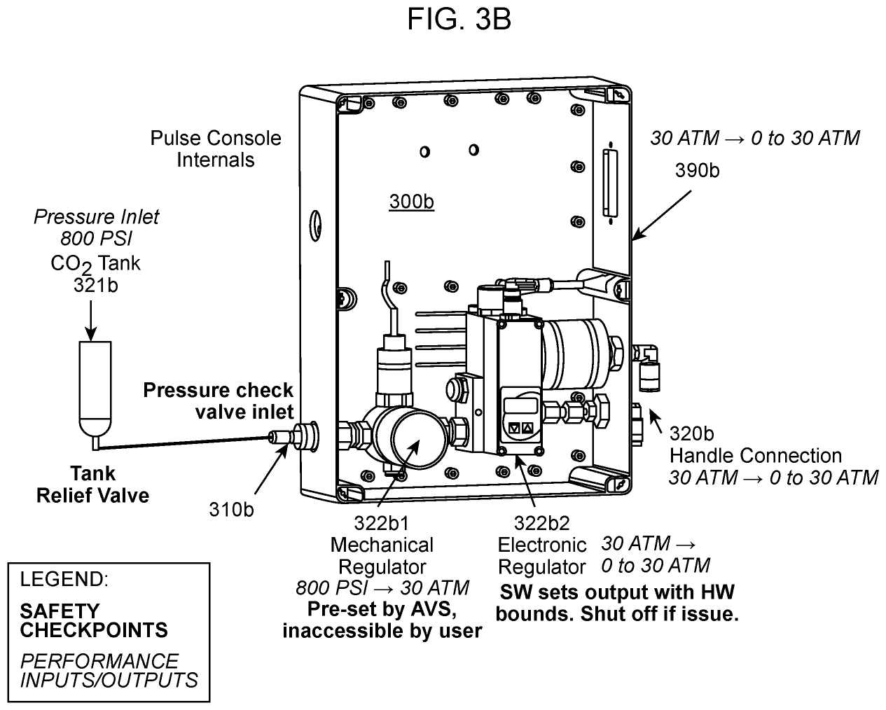

Atherectomy subsystem 160 may further comprise a visualization mechanism, such as a fluoroscope-based or ultrasound-based mechanism, connected to a control unit, such as, for example, an external control unit or unit configured for visualizing aspects of system 100, or connected to a console with display (such as display 330a of console 300a of FIG. 3A) for visualizing the distal end of lateral transmission element 164 or atherectomy tool 169 during an atherectomy procedure, i.e., for visualizing lesion 198 or aspects of vessel 199 as well as for visualizing the location of atherectomy tool 169 relative to lesion 198 or aspects of vessel 199. Lateral transmission element 164 and/or atherectomy tool 169 may comprise radiopaque markers at any convenient location, such as at a distal region or at a distal end, configured to facilitate such visualization. In embodiments, the operator of a system may be able see X-ray imaging while using an embodiment of a system to conduct an atherectomy procedure in order to visualize the atherectomy tool, relative to a lesion, and procedural efficacy. In some instances, feedback, e.g., visual, audio, feel and the like (e.g., vibration of the handle), may be provided to the operator to indicate procedural characteristics such as whether the atherectomy tool has engaged a lesion or crossed a lesion, for example.

As described herein, lateral transmission element 164 may comprise, e.g., a catheter or a guidewire or a combination thereof. Rotational assembly 161 may be attached to the proximal end of lateral transmission element 164 and further configured for controlling the position and orientation of the distal end of the catheter 164 within the vessel, e.g., configured for translating atherectomy tool 169 in a distal direction to further engage occlusion 198. Lateral transmission element 164 of atherectomy subsystem 160 comprises lumen 166, extending through lateral transmission element 164 from a proximal region to a distal region of lateral transmission element 164, for aspirating any removed pieces of occlusions 198 from blood vessel 199, for example, by applying vacuum pressure to aspirate such pieces. Cross sections A and B of FIG. 1B show cross sections of lateral transmission element 164 and atherectomy tool 169 at positions A and B of lateral transmission element 164. In cross sections A and B, lateral transmission element 164 of atherectomy subsystem 160 is depicted as a braided shaft with lumen 166. In other embodiments, lateral transmission element 164 need not be braided and may or may not comprise multiple constituent fibers. When lumen 166 is present, a supporting member, e.g., a guidewire or the like may be present within lumen 166 over all or a portion of the length of lateral transmission assembly, in order to provide further support for atherectomy tool 169. Certain embodiments may comprise a solid shaft for lateral transmission assembly 164, in which embodiments, lumen 166 is not present.

Atherectomy subsystem 160 further comprises a mechanism for detecting and reporting the progress of the atherectomy procedure. Such mechanism may comprise one or more pressure sensors or torque sensors or occlusion sensors or current sensors, connected to a controller, where such mechanism may be configured to detect the extent to which atherectomy tool 169 has pierced a lesion 198, such as a chronic total occlusion.

As described herein, cross sectional diagrams A and B depict cross sections of lateral transmission element 164 at cross sections labeled A and B. At cross section A, lateral transmission element 164 and lumen 166 are shown. At cross section B, lateral transmission element 164 with lumen 166 are shown as well as a cross section of grinding and/or binding material 169a extending outwards radially from lateral transmission element 164 forming atherectomy tool 169, in this case, a rotational grinding burr configured to drill holes in, and expand the diameter of lesions, such as lesion 198 within vessel 199.

Atherectomy subsystem 160 is operated by inserting the distal end of atherectomy subsystem 160 (i.e., distal end of lateral transmission element 164 and atherectomy tool 169) into vessel 199, where atherectomy tool 169 cuts and removes occlusion material 198, such as calcified plaque, from vessel 199. The removed material may be aspirated through lumen 166 and out of vessel 199 and out of the body of the subject, while the progress of the atherectomy procedure may be monitored and reported by a control unit. A visualization mechanism, such a fluoroscopy-based mechanism or an ultrasound-based mechanism, allows an operator, such as a clinician or physician or other operator, to view the distal end of lateral transmission element 164 or atherectomy tool 169, during the procedure, and rotational assembly 161 allows an operator to control the position and orientation of the distal end of lateral transmission element 164 or atherectomy tool 169. For example, aspects of atherectomy subsystem may comprise radiopaque markers.

Amplifier 113 of atherectomy subsystem 160 is releasably connected to handle 111, comprising oscillator 141, and console 110 with potential source 121, which provides the appropriate energy for rotational assembly 161 to rotate atherectomy tool 169 via lateral transmission element 164. Connector 113 receives regulated potential energy such as voltage or pressure or, in other cases, current or flow, from handle 111, and transfers such energy to rotational assembly 161. In some cases, an atherectomy control unit converts the energy to rotational energy. In some cases, such as when handle 111 transmits potential energy in the form of pressurized gas, amplifier 113 receives exhaust gas from rotational assembly 161 and ports it back to console 110 for release into the atmosphere. In some cases, the amplifier 113 connects to various sensors that are present on an atherectomy control unit.

Rotational assembly 161 of atherectomy subsystem 160 is an integral component of the overall system design, and may be configured to enable precise control over the position and orientation of the distal end of the lateral transmission element 164, i.e., precise control over the position and orientation of atherectomy tool 169, within vessel 199 and relative to occlusion 198. Rotational assembly 161 may be designed to allow for both manual and/or automatic control of a linear stage or an advancer responsible for positioning and orienting a lateral position of the distal end of lateral transmission element 164 or atherectomy tool 169, i.e., along arrows 168.

A linear stage present within rotational assembly 161 may be powered by a motor and/or pneumatics and/or manual control by an operator of system 100, providing smooth and precise movement of the distal end of lateral transmission element 164 or atherectomy tool 169. Such a linear stage can be programmed with limits to ensure that the distal end of lateral transmission element 164 or atherectomy tool 169, do not extend beyond a certain range, helping to maintain the safety and efficacy of the atherectomy procedure. Such linear stage may comprise one or more sensors for detecting linear movement of atherectomy tool 169 or may comprise one or more visual references for indicating linear movement of atherectomy tool 169. In embodiments, linear movement of atherectomy tool 169 as measured or indicated at the linear stage may be evaluated or compared, in some cases, in real time during a procedure, against visualization or measurement of aspects of lesion 198. Such comparison enables the detection of whether the distance that the atherectomy tool 169 is translated by the linear stage equals or exceeds the visualized or measured depth of lesion 198. Such measurements or reference distances may be utilized to determine whether the atherectomy tool 169 has moved across lesion 198. In embodiments, the linear stage may be configured such that the linear stage moves the rotational assembly 161. In some embodiments, the linear stage may be configured such that the linear stage moves a motor of the rotational assembly 161 responsible for generating rotation of lateral transmission element 164, which itself is fixed to the lateral transmission element 164. Rotational assembly 161 may be configured to grip, or be fixed to, lateral transmission element 164, such that a linear stage moves both rotational assembly 161 and lateral transmission element 164 together in order to advance atherectomy tool 169. When the linear stage is configured to allow manual control of lateral (i.e., longitudinal) movement of atherectomy tool, the manual linear stage may take the form of a syringe-like structure, wherein the rotational assembly 161 is urged forward relative to an outer housing, for example, upon application of manual force.

To ensure that the distal end of lateral transmission element 164 or atherectomy tool 169, is positioned correctly, rotational assembly 161, as well as other aspects of atherectomy subsystem 160, may be equipped with various feedback sensors. Such sensors provide real-time information on the position, orientation, and movement of the distal end of lateral transmission element 164 or atherectomy tool 169, allowing a control unit to make any necessary adjustments to the position and orientation of the distal end of lateral transmission element 164 or atherectomy tool 169.

The position of the atherectomy tool 169 can be guided using various parameters, such as absolute or relative change in vessel flow or pressure, or based on the rotational characteristics of lateral transmission element 164, such as torque, speed, position, and acceleration. This information can be used by a control unit to optimize the performance of atherectomy subsystem 160, ensuring that occlusions are removed from the vessel as effectively and efficiently and safely as possible.

Atherectomy tool 169 is a grinding burr with a diameter between 0.1 mm to 5 mm and a length between 1 mm to 50 mm. When atherectomy tool 169 comprises a grinding burr, such can be eccentric or concentric with a substantially circular cross section, such as shown in cross section B of tool 169 in FIG. 1B. In embodiments used to treat crossable but narrow lesions, atherectomy tool 169 may comprise a burr located or installed between 1 mm and 15 cm from distal end of lateral transmission element 164. In embodiments used to treat uncrossable lesions, atherectomy tool 169 may comprise a burr located or installed between 0 mm and 20 mm from distal end of lateral transmission element 164.

Atherectomy tool 169, as well as other aspects of atherectomy subsystem 160, such as rotational assembly 161, may be configured such that atherectomy tool 169 is rotated in an orbital and/or a rotational motion. In some cases, the mechanics of rotation are based on the speed, direction, pulsatility, i.e., pulsation, or acceleration of rotation, drive shaft construction, internal mandrel or guidewire, or the position, orientation, mass, or bias of atherectomy tool 169. As described herein, atherectomy tool 169 may be a grinding burr, for example. In embodiments, the mechanics of atherectomy subsystem 160 or atherectomy tool 169 can be controlled to optimize cutting aspects for an atherectomy procedure. In embodiments, the combination of both rotating and orbiting the atherectomy tool has the effect of providing a safeguard with respect to using the atherectomy tool to disrupt lesion tissue only and prevent disruption of healthy tissue. In such cases, the rotation and orbit speeds or other mechanical characteristics (e.g., orbit diameter, etc.) may be selected or otherwise configured such that the movement of the abrasive aspect of the tool is able to engage with lesion tissue, which comprises relatively harder calcified plaque; however, when such abrasive aspect is inadvertently brought into contact with healthy tissue, such as luminal wall tissue, the abrasive aspect of the atherectomy tool is not able to engage with such tissue due to, for example, the relative elasticity of such healthy tissue, minimizing any inadvertent damage to healthy tissue. Such combination of rotational and orbital motion of the atherectomy tool offers an advantage of embodiments of the present invention over traditional techniques at least insofar as embodiments of the present invention provide such safeguard described above with respect to preventing damage to healthy tissue, such as vessel wall 199.

In embodiments, atherectomy tool 169 comprises any abrasive material, binder or mixture for creating a grinding material. Abrasive materials of interest comprise diamond, quartz, aluminum oxide, silicon carbide, zirconia, ceramic alumina, or the like. Binder materials of interest comprise any glass-like or resin bond. In embodiments, abrasive material used to form atherectomy tool 169 is present at a certain diameter that creates a successful grinding effect on calcified tissue, such as lesion 198, such as the specified diameter of atherectomy tool 169 shown in cross section B of FIG. 1B.

In embodiments, lateral transmission element 164, configured to propagate energy to atherectomy tool 169 comprises a suitable braided material such as nitinol, stainless steel, titanium, platinum, aluminum, or the like. Lateral transmission elements of interest may be produced with a core or without a core and may comprise outer and/or inner lubricious coating such as a silicone coating or biocompatible lubrication coating. Lateral transmission elements of interest may be unifiler (comprising a single filament, e.g., an arrangement comprising a single filament) or multifiler (comprising a plurality of filaments, e.g., a braid comprising a plurality of filaments). Lateral transmission elements of interest may be configured to transmit rotational energy from a relatively proximal position of lateral transmission element to a relatively distal position thereof. For example, lateral transmission element may be configured to avoid, or resist, twisting or kinking as it is rotated. That is, lateral transmission element may be configured to resist twisting onto itself or tangling onto itself or kinking itself as it is rotated. In other words, lateral transmission element may be configured to be sufficiently rigid such that it is capable of transmitting rotational energy from a relatively proximal region of the lateral transmission element to a relatively distal region.

Lateral transmission assemblies of interest may have any convenient length, diameter and shape, such as, for example, an outside diameter between 0.002 in to 0.032 in. In some instances, lateral transmission assemblies have a length ranging from 10 cm to 5 m, such as 100 cm to 300 cm. Lateral transmission assemblies may also be referred to as lateral transmission elements. In embodiments in which a lumen or the like is present within lateral transmission element, e.g., for passing over a guidewire, inner diameters of embodiments of lateral transmission elements may be between 0.003 in to 0.087 in. As described, lateral transmission elements of interest, such as lateral transmission element 164, may be configured to create an eccentric or concentric atherectomy tool 169. In some cases, atherectomy tools are created by coating a region of lateral transmission element 164 with grinding and/or binding and/or abrasive material and/or mixtures thereof. In embodiments of lateral transmission elements, metallic particles may be electroplated to the outside of the lateral transmission element 164 in one or more selective locations, e.g., at or around a distal region of lateral transmission element 164.

As described herein, atherectomy subsystem 160 comprises atherectomy tool 169. Atherectomy tool 169 may be any convenient tool for use in modulating lesion 198 or boring a hole through lesion 198 such that pulsatile intravascular lithotripsy subsystem 150 can be used to create cracks in such lesion 198, e.g., along the length of such lesion 198. That is, atherectomy tool 169 may be any convenient tool for modifying lesion 198 such that distal balloon 169 of pulsatile intravascular lithotripsy subsystem 150 can be positioned within lesion 198 such that a pulsatile intravascular lithotripsy treatment can be provided.

Examples of atherectomy tools include rotational, orbital, laser, ultrasound, electrohydraulic lithotripsy (EHL) cavitation emitter or a mechanotransduction tool, etc. In some cases, the atherectomy subsystem 160 comprises guidewire 167 and utilizes mechanotransduction of guidewire 167 to create a bore through an occlusion or lesion or chronic total occlusion 198.

Depending on the type of occlusion 198 and type of atherectomy tool 169 employed, atherectomy tools may be configured to grind within a lesion or to cross a lesion. In some cases, atherectomy tools are configured to create a new channel within an occlusion or to drill a hole in an occlusion. In embodiments, atherectomy tool 169 is a burr, such as a grinding burr. As described, atherectomy subsystem 160 may be employed to create a channel, hole or other space within a lesion or occlusion or other obstruction of a vessel or luminal tissue, such as a lesion comprising calcified plaque, such that distal balloon 159 of pulsatile intravascular lithotripsy subsystem 150 can be positioned within this new channel to further disrupt lesion 198. That is, atherectomy subsystem 160 may be employed to create a volume in which distal balloon 159 can fit within lesion to provide pulsatile intravascular lithotripsy treatment, further disrupting the lesion.

Referring again to FIG. 1A, two representations of the same vessel wall 199 with the same occlusion or lesion 198 are depicted on the right-hand side of the figure. Such vessel wall 199 with occlusion or lesion 198 are the same in the top and bottom representations and are duplicated in order to illustrate how each of the atherectomy subsystem 160 and pulsatile intravascular lithotripsy subsystem 150 interface with the same vessel wall 199 with the same occlusion or lesion 198. As reviewed herein, occlusion or lesion 198, such as a chronic total occlusion, may comprise calcified plaque (CP). In some embodiments, atherectomy subsystem 160 further comprises one or more sensors, such as sensor 162, configured to sense electrical changes, such as a current or potential, or to sense mechanical changes, such as changes in speed or torque applied by atherectomy tool, resulting from atherectomy tool 169 penetrating occlusion 198.

As described herein, atherectomy subsystem 160 of system 100 includes connector 113 and rotational assembly 161. Rotational assembly 161 is operably connected, via connector 113, to console unit 120 via handle 111 and is configured to utilize potential energy 122 transmitted from console unit 120 to produce rotational energy. That is, rotational assembly 161 is configured to transduce energy transmitted from console unit 120 via handle 111 and connector 113 into rotational energy for use by atherectomy tool 169. Rotational assembly 161 may comprise a motor, such as an air motor or turbine-based motor, for example, or an electric motor. In some cases, rotational assembly 161 and connector 113 are configured such that rotational assembly 161 draws electric potential from handle 111, via connector 113.

As described herein, atherectomy subsystem 160 of system 100 includes lateral transmission assembly 164. Lateral transmission assembly 164 is configured to propagate rotational energy from rotational assembly 161 to atherectomy tool 169 such that atherectomy tool 169 can be used to create a hole or bore or otherwise disrupt occlusion 198 such that distal balloon 159 of pulsatile intravascular lithotripsy subsystem 150 may be subsequently inserted into lesion 198. Lateral transmission assembly 164 comprises a flexible drive shaft for use propagating rotational energy to atherectomy tool 169. Lateral transmission assembly 164 may be configured to be sufficiently flexible to traverse a vessel to an occlusion and simultaneously sufficiently rigid such that rotational energy is transmitted without causing the lateral transmission assembly to twist or kink. Lateral transmission assembly 164 is located distal to rotational assembly 161 and proximal to atherectomy tool 169. Lateral transmission assembly 164 comprises a guidewire lumen with guidewire 167 present therein in part for guiding atherectomy tool 169 to occlusion 198 within vessel 199 and in part for imparting stability to the lateral transmission assembly 164 while propagating rotational energy to atherectomy tool 169.

In some cases, lateral transmission assemblies may comprise a fluid bearing configured for, in part, dissipating heat, e.g., caused by rotation of one or more aspects of the lateral transmission assembly, such as rotation of a guidewire present in fluid, or rotation of a catheter around a guidewire present in fluid or the like.

Embodiments of atherectomy subsystem 160 comprise an advancer integrated as part of rotational assembly 161 configured to advance and retract atherectomy tool 169 in distal and proximal directions. Such distal and proximal advancing and retracting is illustrated by arrows 168 that show how distal tool 169 can be advanced towards, and retracted from, occlusion 198. In some cases, the advancer is configured to pulse atherectomy tool 169 into and out of an occluded lesion 198; i.e., such that atherectomy tool 169 pecks at one or more surfaces of occlusion 198.

Referring now to FIG. 1C, this figure provides a more detailed view of certain aspects of pulsatile intravascular lithotripsy subsystem 150 of system 100. Referring now to FIGS. 1A and 1C, pulsatile intravascular lithotripsy subsystem 150 includes proximal connector 151 having a proximal port 151a for operably connecting to a pneumatic output of handle 111, a proximal flexible tube 151c coupled to a distal port 151b of proximal connector 151; a distal catheter shaft 154 having an angioplasty balloon 159, such as a composite balloon, located at a distal end thereof; and a Y-connector 156 connecting the distal end of the proximal flexible tube 151c to the proximal end of the distal catheter shaft 154. Proximal connector 151 may also be referred to as a connector or an amplifier. Also shown is optional valve 156a positioned between the distal end of the proximal flexible tube 151c and the Y-connector 156.

When present, the proximal flexible tube 151c acts as a strain relief between the proximal connector 151 and the Y-connector 156, distal catheter shaft 154, and angioplasty balloon 159. When present, valve 156a may be employed to introduce fluid into the liquid passageway or passageways of pulsatile intravascular lithotripsy subsystem 150. In some instances, valve 156a is not present. For example, as described herein, pulsatile intravascular lithotripsy subsystem 150 may be a closed or sealed system that is provided to a user prefilled, in some cases with a suitable contrast agent containing liquid. In such instances, valve 156a may not be provided since fluid priming would not be required to use the pulsatile intravascular lithotripsy subsystem 150. In other instances, valve 156a is a tee connector with a one-way valve protruding from one of the tee connectors. Such one-way valve 156a allows for easy priming of catheter 154 and balloon 159. In other embodiments comprising tee connectors, the connections to the proximal flexible tube 151c and Y-connector 156 are made with a rotating Luer to facilitate easy positioning of the catheter 154 and valve 156a with respect to other aspects of system 100.

Additional details regarding pulsatile intravascular lithotripsy subsystems that may be incorporated into embodiments of the present invention are provided in U.S. Pat. No. 11,464,949, United States Published Patent Application Publication No. 20200046949 as well as pending PCT Application Serial No. PCT/US2020/055458 and pending PCT Application Serial No. PCT/US2022/014785; the disclosures of which are herein incorporated by reference.

As described herein, an output of handle 111 comprising oscillator 141 is operably connected to pulsatile intravascular lithotripsy subsystem 150. In particular, an output of handle 111 is connected to an input of proximal connector 151. Proximal connector 151 is configured to transduce potential energy such as, for example, pneumatic pressure (i.e., a first pulse energy), generated by oscillator 141, to a second potential energy such as, for example, hydraulic pressure (i.e., a second pulse energy). In certain embodiments, systems may include multiple connectors with individual connectors corresponding to each of a plurality of handles. In embodiments, proximal connector 151 comprises an interface for releasably operably connecting to an interface of handle 111 with identical shape and/or connections and/or interlocking elements as connector 113 of atherectomy subsystem 160, such that each of connector 113 and proximal connector 151 can be interchangeable operably connectable to handle 111.

An output of proximal connector 151 is operably connected to catheter 154 to enable potential energy output from proximal connector 151 (i.e., second pulse energy) to be input into catheter 154 (e.g., one or more fluidic channels within catheter 154, such that a volume within proximal connector 151 is in fluidic communication with catheter 154). In some cases, pulsatile intravascular lithotripsy subsystem 150 comprises more than one catheter 154, or catheter 154 comprises more than one fluidic channel that is internal or external to catheter 154, such that, in either case, different sections of distal balloon 159 or catheter 154 may be independently operated. For example, distal balloon 159 may comprise a plurality of different balloons, each independently pressurized or inflated and deflated, such as, for example, different aspects of a heart-tissue-conforming element.

Referring again to FIG. 1A, in pulsatile intravascular lithotripsy subsystem 150 of system 100, catheter 154 includes a lumen dedicated for a guidewire 157 so that catheter 154 can be navigated to a cardiovascular tissue treatment site 198 via a standard over-the-wire (OTW) guidewire approach. Alternatively, in embodiments, catheter 154 includes one or more guidewire ports for guidewire 157 such that catheter 154 and balloon 159 can be navigated to a cardiovascular tissue treatment site 198 via a standard rapid exchange (RX) guidewire approach. In other embodiments, pulsatile intravascular lithotripsy subsystem 150 is configured for use with a monorail approach.

Pulsatile intravascular lithotripsy subsystem 150 may be configured to include a guidewire exit port 157a for a proximal region of a guidewire 157 threaded through a lumen or a guidewire channel or a ring or a loop of catheter 154. Pulsatile intravascular lithotripsy subsystem 150 may include a guidewire exit port 157b opposite guidewire entrance port and present at a relatively distal region of intravascular pulsatile lithotripsy subsystem 150, e.g., distal to distal balloon 159. In embodiments, a guidewire exit port 157b may be oriented in a manner that is substantially parallel to the longitudinal axis of catheter 154, and therefore parallel to the longitudinal axis of a guidewire channel within catheter 154, in order to avoid any unnecessary bends in guidewire 157. As described herein, system 100 may be configured with respect to guidewire 157 and the pulsatile intravascular lithotripsy subsystem 150 so that system 100 is an over-the-wire (OTW) or rapid exchange (RX) or monorail system or the like.

In some cases, atherectomy subsystems and pulsatile intravascular lithotripsy subsystems may comprise the same guidewire. That is, with reference to FIGS. 1A-C, guidewire 157 and guidewire 167 may be the same guidewire. In such cases, guidewire 167 is initially inserted into vessel 199 and used to guide atherectomy subsystem 160 into a desired location such that an atherectomy procedure is performed; subsequently, atherectomy subsystem 160 is removed from vessel 199, while guidewire 167 remains in place within vessel 199; guidewire 167 is then used to guide pulsatile intravascular lithotripsy subsystem 150 into a desired location such that a pulsatile intravascular lithotripsy procedure cab be performed. In such cases, guidewire 167 is inserted into vessel 199 a single time and utilized by each of the atherectomy subsystem 160 and pulsatile intravascular lithotripsy subsystem 150. Embodiments of the present invention offer advantages relative to traditional approaches at least insofar as a single guidewire, such as guidewire 167, can be used with both atherectomy subsystem 160 and pulsatile intravascular lithotripsy subsystem 150, enabling a simplified and streamlined process for using each subsystem to treat lesion 198.

In embodiments as illustrated schematically in FIG. 1A, distal balloon 159 is present at a distal region of catheter 154. In system 100, distal balloon 159 is configured to engage lesion 198 present within vessel wall 199, such that application of pressure to distal balloon 159 causes calcium deposits of lesion 198 to crack or to otherwise be disrupted within vessel 199. Distal balloon 159 may comprise non-compliant/compliant material, as described herein. In embodiments, the space within lesion 198 in which distal balloon 159 is positioned was created using atherectomy tool 169 of atherectomy subsystem 160. Any convenient balloon, or plurality of balloons or heart-tissue-conforming element, for example, may be employed for distal balloon 159. Suitable balloons include, but are not limited to, standard angioplasty balloons, such as compliant and non-compliant angioplasty balloons. In one embodiment, the balloon is a composite balloon that includes two distinct layers, which layers include a non-compliant layer and compliant layer.

In certain embodiments, distal balloon 159 may comprise more than one balloon, each configured to be independently operable, i.e., independently pressurized and depressurized, by, for example, being pressurized by hydraulic pressure transmitted via a separate fluidic channel of catheter 154.

In embodiments, as distal balloon 159 is repeatedly pressurized and depressurized, fluid, e.g., blood, is allowed to perfuse past distal balloon 159 even while applying pulsatile energy to tissue, e.g., cardiovascular tissue, at treatment site 198 (i.e., pulsatile inflation allows fluid to perfuse past balloon during parts of a pulsatile cycle when the balloon is substantially deflated). Such a configuration enables blood flow past distal balloon 159, enabling extended treatment times.

Embodiments of pulsatile intravascular lithotripsy subsystem 150 may be further configured to isolate, suspend or otherwise filter fluid perfusing past distal balloon 159 in order to, for example, protect surrounding vasculature from distal emboli. In some cases, pulsatile intravascular lithotripsy subsystem 150 comprises a filter located distal to distal balloon 159, which may be attached to distal balloon 159 and/or catheter 154 and/or guidewire 157.

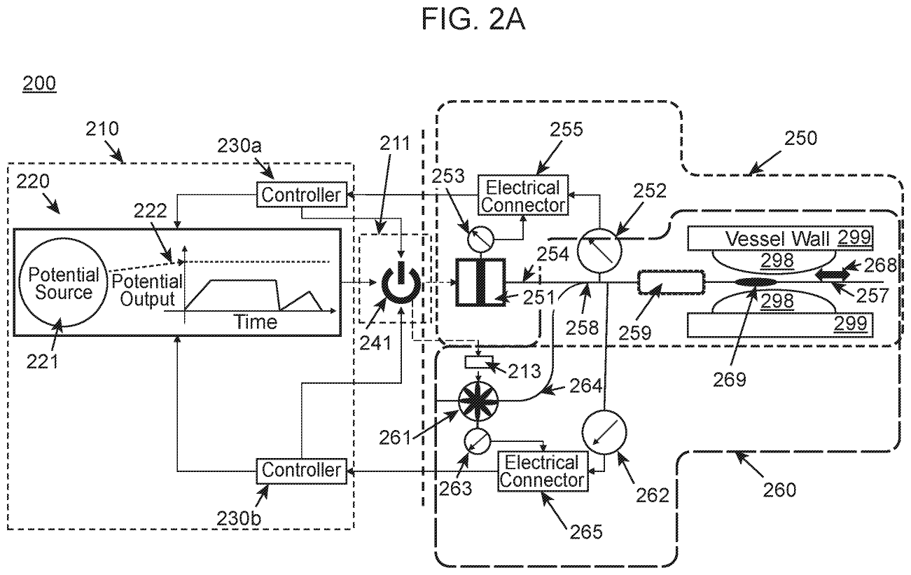

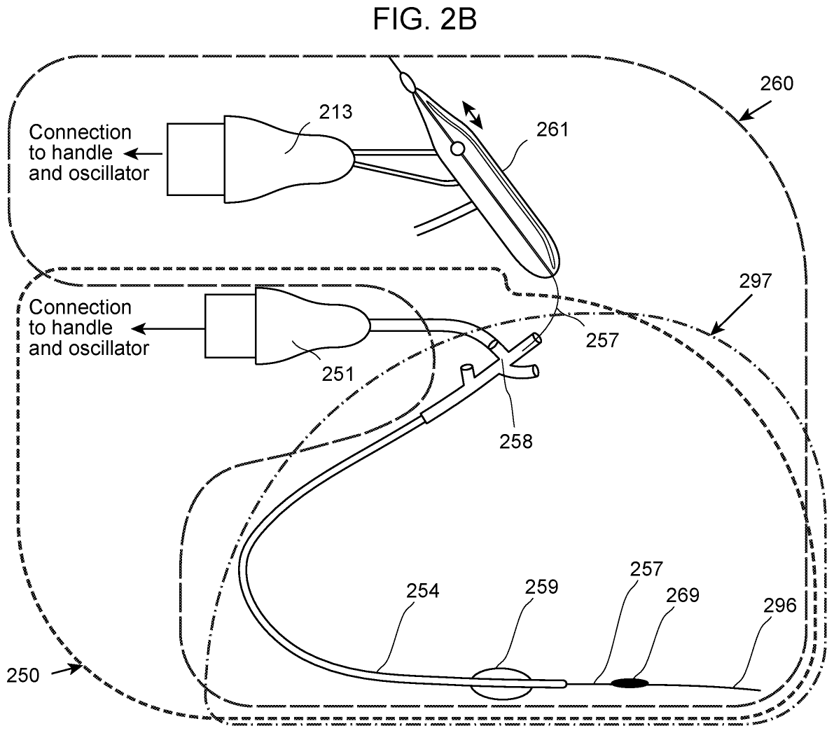

In embodiments, one or more filters may be attached to a region, such as a distal region or the distal end of guidewire 157, 167 or microcatheter 154 or lateral transmission assembly 164. Such filter may comprise any convenient filter configured to receive and collect dislodged pieces, or fractured parts, of lesion 198, e.g., calcified plaque, such as emboli, as such pieces are dislodged or disrupted during operation of the atherectomy subsystem or the pulsatile intravascular lithotripsy subsystem, immobilizing and preventing such pieces or emboli from traveling throughout the vascular system. While, as described herein, FIGS. 1A-C present a combined atherectomy and pulsatile intravascular lithotripsy system with separate tools, and FIGS. 2A-B present a combined atherectomy and pulsatile intravascular lithotripsy system with integrated tools, as described below, one or more distal filters is not limited to one or the other of such types of embodiments nor limited to one or the other of embodiments of atherectomy subsystems and pulsatile intravascular lithotripsy subsystems. Similarly, embodiments of the present invention may be configured such that a distal region of the atherectomy subsystem and/or the pulsatile intravascular lithotripsy subsystem are configured to aspirate fluid, such that dislodged pieces, or fractured parts, of a lesion, e.g., calcified plaque, such as emboli, are aspirated, removing them from the vasculature and preventing them from traveling throughout the vasculature.

In instances, aspects of system 100, such as, for example, aspects of console assembly 110 (e.g., controllers 130a, 130b or potential source 121), handle 111, oscillator 141, pulsatile intravascular lithotripsy subsystem 150 (e.g., proximal connector 151 or distal balloon 159) or atherectomy subsystem 160 (e.g., rotational assembly 161 or connector 113 or lateral transmission assembly 164 or atherectomy tool 169) may be configured to be reusable. In instances, aspects of system 100, such as, for example, catheter 154 or distal balloon 159 or lateral transmission assembly 164 or atherectomy tool 169 may be configured to be used a single time. That is, such elements may be disposable. The terms “reusable” and “disposable” as employed here and elsewhere throughout the description are used for convenience in describing an embodiment of the invention, such as that illustrated in FIGS. 1A-C as well as that illustrated in FIGS. 2A-B, described in detail below. However, the invention is not so limited. As such, any part of the system may be configured for one time use or for use multiple times, as desired.

Now referring to FIGS. 2A-B, in accordance with another embodiment, another combined atherectomy and pulsatile intravascular lithotripsy platform, in this case, with combined tools, is described. In FIGS. 2A-B, elements having the same or similar reference numerals have the same or similar features as corresponding elements in FIGS. 1A-C, unless explicitly stated otherwise. Descriptions of elements already described above in connection with FIGS. 1A-C are not repeated below.

System 200 comprises integrated atherectomy subsystem 260 and pulsatile intravascular lithotripsy subsystem 250 with combined tool 297 (labeled in FIG. 2B). Such subsystems are integrated in a relatively distal region from connection 258. Embodiments integrated as such enable a user, such as, for example, an operator or a clinician, to insert a single catheter assembly into a vessel, connect atherectomy subsystem 260 to handle 211, perform an atherectomy procedure using the integrated atherectomy subsystem 260, disconnect atherectomy subsystem 260 from handle 211, connect pulsatile intravascular lithotripsy subsystem 250 to handle 211 and perform a pulsatile intravascular lithotripsy procedure. That is, use of such an embodiment with integrated tools enables a user to insert a catheter assembly of integrated atherectomy subsystem 260 and pulsatile intravascular lithotripsy subsystem 250 (i.e., combined tool 297) into a vessel only once over the course of an atherectomy and pulsatile intravascular lithotripsy treatment. Avoiding inserting multiple assemblies or catheters or guidewires into a vessel offers advantages of reducing procedure time and complications.

In certain cases, as described in greater detail herein, the atherectomy tool 269 is combined with guidewire 257 used for pulsatile intravascular lithotripsy. That is, atherectomy tool 269 may be present on guidewire 257 operably connected to distal balloon 259 of pulsatile intravascular lithotripsy subsystem 250. In such cases, guidewire 257 may be referred to as atherectomy wire. Similar to conventional guidewires, atherectomy wire 257 has a proximal end (i.e., relatively nearer to connector 251 and rotational assembly 261) and distal end (i.e., relatively closer to distal balloon 259 and atherectomy tool 269). In embodiments, the distal end of guidewire 257 can be coated with a grinding material to create a burr that forms atherectomy tool 269. In other cases, a burr forming atherectomy tool 269 can be assembled onto the shaft of guidewire 257.

Depending on the axial location of atherectomy tool 269, this tool can be used in two ways: (1) to cross narrow lesions 298 within a vessel 299 and then grind within that lesion or (2) to create a new channel by butting up against a narrow or occluded vessel 299 and grinding or drilling a hole. The proximal end of guidewire 257 can be backloaded onto pulsatile intravascular lithotripsy catheter 254, or regions thereof, to provide support for catheter 254. In turn, pulsatile intravascular lithotripsy catheter 254 provides support for atherectomy tool 269 during rotation or orbit of such tool and provides a containment area for guidewire 257 to rotate. Lateral transmission assembly 264 comprises guidewire 257 as well as catheter 254. That is, in a region distal from connection 258, lateral transmission assembly 264 comprises guidewire 257 present within catheter 254, and in a region proximal from connection 258, lateral transmission assembly 264 comprises guidewire 257 separate from, or not enclosed within, catheter 254.

In a relatively distal direction from connection 258, lateral transmission assembly 264 comprises guidewire 257 integrated with catheter 254, such that guidewire 257 can provide support for catheter 254, and catheter 254 can provide support for atherectomy tool 269 as well as a containment area for lateral transmission assembly 264 to rotate or orbit.

System 200 is an embodiment of a combined atherectomy and pulsatile intravascular lithotripsy platform with combined tools in part because aspects of pulsatile intravascular lithotripsy subsystem 250 and atherectomy subsystem 260 are integrated together at, and distal to, connection 258. As described, in a relatively distal direction from connection 258, catheter 254, lateral transmission assembly 264, guidewire 257 are integrated, such that guidewire 257 may be present within or otherwise attached to catheter 254. In some embodiments, lateral transmission assembly 264 comprises guidewire 257 and such guidewire is configured to rotate, thereby transmitting energy from rotational assembly 261 to atherectomy tool 269, within catheter 254. That is, rotational assembly 261 rotates guidewire 257 within catheter 254. In some cases, catheter 254 and guidewire 257 are configured to form a fluid bearing to facilitate rotation of guidewire 257, in particular to facilitate heat dissipation associated with rotation of guidewire 257.

As illustrated in FIGS. 2A-B, similar to FIGS. 1A-C, the atherectomy lateral transmission assembly 264 connects to rotational assembly 261, which provides the rotational motion of atherectomy tool 269. As described herein, rotational assembly 261 may comprise a motor, e.g., a motor configured to rotate lateral transmission assembly 264. This rotational motion is used to drill areas of lumen 299 that are too narrow for a pulsatile intravascular lithotripsy catheter 254 and distal balloon 259 to cross. Such drilling is performed to expand lumen 299 within lesion 298, such as, for example, an occlusion and/or calcifications, such that pulsatile intravascular lithotripsy can be performed on calcifications 298 with distal balloon 259 of pulsatile intravascular lithotripsy subsystem 250. In embodiments, such as system 200, that are embodiments of a combined atherectomy and pulsatile intravascular lithotripsy platform with combined tools, after a hole is drilled or expanded in lesion 298, the combined tools, comprising atherectomy tool 269, distal balloon 258, guidewire 257, catheter 254 and lateral transmission assembly 264 can be advanced by moving them along longitudinal axis 268 such that distal balloon 259 can be positioned with respect to lesion 298 to apply pulsatile energy to lesion 298, cracking or otherwise breaking up or disrupting calcified plaque therein, from the hole drilled or expanded by atherectomy tool 269. In such case, distal balloon 259 can be positioned to apply pulsatile energy to lesion 298 from the hole drilled or expanded by atherectomy tool 269 without the need to remove atherectomy subsystem 250 from lumen 299. Instead, distal balloon 259 is positioned into or near lesion 298 by, for example, moving catheter 254 with distal balloon 259 in a relatively distal direction within vessel 299, and pulsatile intravascular lithotripsy 250 is engaged to apply pulsatile energy to lesion 298.

In embodiments, atherectomy subsystem 260 is detached from handle 211, and pulsatile intravascular lithotripsy subsystem 250 is attached to handle 211 without the need to reinsert additional or different tooling into lumen 299. That is, since pulsatile intravascular lithotripsy subsystem 250 and atherectomy subsystem 260 are integrated together in a region distal to connection 258, there is no need to insert additional tooling into vessel 299 when transitioning from an atherectomy procedure to a pulsatile intravascular lithotripsy procedure.

FIG. 2B provides a schematic of a combined tool 297 of system 200 comprising aspects of atherectomy subsystem 260 and aspects of pulsatile intravascular lithotripsy subsystem 250, in accordance with an embodiment of the invention. Atherectomy tool 269 is present at a distal region of guidewire 257, and is configured to receive power from rotational assembly 261 in order that atherectomy tool 269 can drill a hole in lesion 298 within vessel 299. In the embodiment depicted, lateral transmission assembly comprises guidewire 257. Rotational assembly 261 is operably connected to connector 213, itself operably connected to handle 211. As described herein with respect to connector 113 of FIGS. 1A-C, connector 213 may similarly be a pass-through device configured to pass potential energy from output of handle 211 to rotational assembly 261.

In combined tool 297, atherectomy tool 269 is directly connected to distal balloon 259 and catheter 254 of the pulsatile intravascular lithotripsy subsystem 250, such that guidewire 257 may be housed within catheter 254 over a portion of the lateral extent of guidewire 257. Distal balloon 259 receives power transmitted from proximal connector 251 via catheter 254 for use performing pulsatile intravascular lithotripsy. Using combined tool 297 may involve drilling a hole, or expanding an opening, in lesion 298 in vessel 299 using atherectomy tool 269 of atherectomy subsystem 260. Once such hole is drilled or expanded, catheter 254 of the intravascular pulsatile lithotripsy subsystem 250 can be advanced in a distal direction within vessel 299 such that distal balloon 259 interfaces with lesion 298 via the newly drilled or expanded hole. Once distal balloon 259 can be arranged in a desired location within such hole in lesion 298, pulsatile intravascular lithotripsy can be commenced by transmitting pulse energy from proximal connector 251 to distal balloon 259. That is, an important feature of combined tool 297 is that instead of atherectomy tool 269 utilizing a separate wire or catheter mechanism, atherectomy tool 269 is present on guidewire 257 that itself is connected to distal balloon 259, such that atherectomy subsystem 260 need not be removed from vessel 299 before commencing a pulsatile intravascular lithotripsy procedure using pulsatile intravascular lithotripsy subsystem 250. In such embodiments, guidewire 257 with atherectomy tool 269 provides support to catheter 254 and balloon 259, and vice versa.

In embodiments with combined tools, such as in combined tool 297, system 200 is a unified atherectomy and pulsatile intravascular lithotripsy system. In embodiments, the atherectomy subsystem 260 and the pulsatile intravascular lithotripsy subsystem 250 comprise a unitary tool 297. For example, the atherectomy subsystem 260 and the pulsatile intravascular lithotripsy subsystem 250 may comprise a common distal region, such as, for example, a region of system 200 that is distal to connection 258. In some cases, both the atherectomy subsystem 260 and the pulsatile intravascular lithotripsy subsystem 250 comprise a common guidewire 257. In system 200, the lateral transmission assembly is guidewire 257.

System 200 may be configured such that system 200 is an over the wire (OTW) system. For example, in some cases, a guidewire is present over a substantial length of the atherectomy subsystem 260 and the pulsatile intravascular lithotripsy subsystem 250. In other instances, system 200 may be configured such that system 200 is a rapid exchange (RX) system. For example, in some cases, guidewire 257 is present over only a distal region of the atherectomy subsystem 260 and the pulsatile intravascular lithotripsy subsystem 250. In still other instances, embodiments of systems may be configured as a monorail system. That is, embodiments of systems may utilize a guidewire in a monorail technique.

In embodiments of systems with combined tools comprising a common guidewire shared between the atherectomy subsystem and the pulsatile intravascular lithotripsy subsystem, a distal region of the guidewire may be coated with a grinding material, and such grinding material coating may comprise an atherectomy tool, e.g., a distal burr. In some cases, the grinding material coating has a predetermined diameter selected based on treatment efficacy. In other cases, the grinding material coating has a predetermined diameter selected based on a diameter of a distal balloon of the pulsatile intravascular lithotripsy subsystem. In embodiments, such predetermined diameter is selected such that a distal balloon of the pulsatile intravascular lithotripsy subsystem can be inserted into a hole created by the grinding material.

In other cases of systems with combined tools comprising a common guidewire shared between the atherectomy subsystem and the pulsatile intravascular lithotripsy subsystem, the pulsatile intravascular lithotripsy subsystem comprises a guidewire lumen. In such cases, the common guidewire may be present within the guidewire lumen of the pulsatile intravascular lithotripsy subsystem. In some embodiments, the system may further comprise a filter present on the guidewire, distal to the atherectomy tool, e.g., such as region 296 comprising a filter. In embodiments, such filter is configured to protect vasculature from distal emboli, e.g., associated with drilling into and/or applying pulsatile energy to a lesion.

In other embodiments of the invention that do not comprise combined tools or combined distal regions, the atherectomy subsystem and the pulsatile intravascular lithotripsy subsystem have separate distal regions. In such embodiments that do not comprise combined tools, a catheter component of the pulsatile intravascular lithotripsy subsystem is separate from the atherectomy subsystem.

An embodiment of a method for using a system of the present invention with combined tools, such as, for example, system 200, comprises: inserting a sheath/guide catheter into a vessel 299; inserting guidewire 257 with atherectomy tool 269 into vessel 299; loading pulsatile intravascular lithotripsy balloon 259 and catheter 254 over guidewire 257 such that guidewire 257 provides stability to catheter 254; loading guidewire 257 into rotational assembly 261 that is an atherectomy motor; locking guidewire 257 onto rotational assembly 261; connecting connector 213 into handle 211; running therapy comprising an atherectomy procedure by holding a handle button or the like and advancing guidewire 257 and therefore advancing atherectomy tool 269; generating sufficient luminal gain to allow the pulsatile intravascular lithotripsy balloon 259 to pass into lesion 298; pushing guidewire 257 further into lesion 298 in a distal direction such that atherectomy tool 269 advances past the treatment site; that is, in a location where the atherectomy tool was used to expand the lesion; advancing the pulsatile intravascular lithotripsy balloon 259 across such treatment site; disconnecting atherectomy connector 213 from handle 211 and connecting pulsatile intravascular lithotripsy proximal connector 251 to handle 211; and treating with pulsatile intravascular lithotripsy subsystem 250, i.e., transmitting pulsatile energy to lesion 298 via distal balloon 259. Atherectomy connector 213 can be disconnected from handle 211, and proximal connector 251 can be connected to handle 211 because each such element comprises an interface with features, such as, for example, shape or interlocks or connectors or other alignment features, that enable each of connector 213 and proximal connector 251 to be interchangeable operably connectable to the handle 211.

### Console:

Console devices of embodiments of the invention may vary. In some instances, the console device is a compact, standalone unit designed for ease of use and versatility. The device may include an enclosure that protects the internal components and provides a secure and stable base for operation. The enclosure may be designed to be durable and resistant to damage from handling and environmental factors, such as dust and moisture. The console device can be configured to be attached to an IV pole, e.g., using clamps, such as using two clamps that securely hold the device in place. In such embodiments, the clamps can be adjusted to fit a variety of IV pole sizes, ensuring compatibility with a wide range of equipment. In addition, the console device may include table stands that provide stability when the device is used on a flat surface.

In some instances, the console device includes a gas inlet port that allows for the connection of high-pressure gas, such as compressed gas, carbon dioxide, nitrogen, or the like. The gas inlet port may be designed to ensure a secure and leak-free connection to the source of gas. The console device may include a pressure reducing regulator that regulates the incoming high-pressure gas to a desired output pressure. The regulator may be designed to provide precise and consistent pressure regulation, ensuring the safe and effective delivery of the regulated gas. Console devices of embodiments of the invention may include a proportional or electronic control valve that outputs either a desired pressure or flow rate. The valve may be designed to provide precise and accurate control of the output pressure or flow rate, ensuring the safe and effective delivery of the regulated gas.

As the console device uses power to operate, power may be provided through a power inlet cable connector. The cable connector may be designed to be secure and resist damage from handling and environmental factors. The console device may include harnesses that provide connectivity to the internal components to ensure a secure and reliable electrical connection. Where desired, the console device may include a voltage converter that converts the incoming power to the voltage required by the internal components. The voltage converter may be designed to be efficient and reliable, ensuring consistent operation of the console device.

In embodiments, the console device may include fluid management components to handle fluid output, e.g., of pressurized sterile saline. This may include a sterile saline reservoir, a pump for pressurizing the saline, and tubing for directing the saline to the treatment area. The fluid management components may be designed to ensure the sterile and safe delivery of the pressurized saline, ensuring the overall safety and effectiveness of the treatment process. The fluid management system may also be designed to be easy to use and maintain, ensuring efficient operation and maintenance of the console device.

The console device may utilize various sensors to monitor the performance of the console device and ensure the safe delivery of potential energy, such as, for example, high-pressure gas. Pressure transducers, for example, may be used to measure the pressure within the system and can be used to gauge the remaining amount of gas. Optical or other types of flow sensors may be employed to detect changes in the flow of gas, and temperature sensors monitor the temperature of components to prevent overheating or cold environments. Humidity sensors, e.g., inside and/or outside of the console, may be used to ensure that the humidity levels remain within acceptable ranges, to detect a leak, and/or to detect condensation buildups due to the exhaust gases. Additionally, connection sensors may be used to detect when a tether, e.g., as described herein, is properly connected to the console, as well as when it is disconnected. In embodiments, this information may be crucial for ensuring that the console operates as intended and can stop the flow of gas if a disconnection is detected. By using a combination of these sensors, in embodiments the console is able to monitor its own performance and make adjustments to ensure the safe and effective delivery of potential energy, such as, for example, high-pressure gas. The sensors may be configured to provide valuable feedback to the system, enabling it to operate more efficiently and effectively, while also protecting the user and patient from potential risks.

The console device may include a display screen that provides real-time information to the user, such as pressure and flow rate readings, treatment duration, and other relevant information. The display screen may be easy to read and provide clear and concise information to the user. In embodiments, the console device is equipped with a display screen that presents information to the user, including, for example, the current treatment type, intensity, and duration. The console device may also be equipped with software that runs programs that determine the type, intensity, and duration of treatment, and this software can be preset, user-adjusted, based on sensor input, or based on artificial intelligence, machine learning or the like.

The console device may be further equipped with an adjusting component that can alter the device's settings based on user feedback or other sensor input. This component can be configured to allow the console device to continuously monitor and adjust its performance to ensure the regulated gas output remains within safe and effective parameters.

The software of the console device may be designed to take various sensor inputs and adjust treatment settings in real-time. The software may be configured to continuously monitor the input from various sensors, such as those measuring gas pressure, flow rate, imaging, vessel pressure and/or flow, and patient vital signs. The software then uses this input to adjust the treatment settings, such as the intensity and duration of the regulated gas output. The software can be programmed with various presets for different types of treatments, and these presets can be modified by the user or based on artificial intelligence algorithms. Such artificial intelligence algorithms can analyze the sensor input to determine if the treatment settings need to be adjusted to ensure the regulated gas output remains within safe and effective parameters. Additionally, the software can also incorporate user feedback, such as manual adjustments made by the caregiver or the patient, to further refine the treatment settings. The software will incorporate this feedback and adjust the treatment settings accordingly to provide the most effective and efficient treatment.

The console device may be configured to adjust itself based on feedback or feedforward models, providing a highly adaptable and responsive system for supplying potential energy, such as, for example, administering regulated gas. Feedback models refer to adjustments made by the console device based on actual sensor inputs and performance results. For example, the console device may monitor the gas pressure and adjust the regulating component to maintain the pressure within a safe and effective range. The software can also analyze the sensor input and make real-time adjustments to the treatment settings, such as the intensity and duration of the regulated gas output. Feedforward models refer to adjustments made by the console device based on predicted future inputs or performance results. For example, the software can be programmed with artificial intelligence algorithms that analyze sensor data and predict potential future changes in the patient's condition. Based on these predictions, the console device can pre-emptively adjust the treatment settings to ensure the regulated gas output remains within safe and effective parameters.

In embodiments, the console device can communicate with cloud-based servers or cloud-based data or otherwise previously stored data to improve or recommend treatment profiles. This communication allows the device to access a wealth of information and knowledge to provide a more personalized and effective treatment experience for the patient. In embodiments, the console device can communicate with a cloud-based database or electronic medical records system to access a patient's previous treatment history and other relevant information, such as their medical history, current medications, and vital signs. This information can then be used to tailor the treatment settings to the individual patient, providing a more personalized and effective treatment experience. The console device may be configured to access previously stored data to improve its performance over time. For example, the device can analyze past treatment data to identify trends and patterns, and this information can be used to improve the accuracy and efficiency of future treatments.

In some instances, the console device can be configured to communicate with other devices, such as remote monitoring devices, to access additional information that can be used to improve treatment profiles. For example, the console device can be configured to receive data from a wearable device that measures the patient's vital signs, and this information can be used to adjust the treatment settings to ensure the regulated gas output remains within safe and effective parameters. In embodiments, the console device can communicate with a wide range of external devices to be programmed by, receive information from, or provide information to. This communication is supported through a variety of wired and wireless communication protocols, including RS232, USB, Ethernet, Wi-Fi, and Bluetooth. Additionally, the Console device can support other communication platforms such as Zigbee, NFC (Near Field Communication), MQTT (Message Queue Telemetry Transport), MODBUS, and CAN (Controller Area Network), depending on the specific needs and requirements of the device. These communication protocols allow the console device to be highly flexible and adaptable, ensuring compatibility and communication with a wide range of external devices.

The Console device may include connectors, such as a pair of mating connectors, that allow for secure and reliable connection between the console device and a tether. In such instances, one connector may be fixed on the outside of the console, while the other is connected to the tether. The connectors may mate two gas ports (inlet and exhaust), as well as an electrical connector that provides power and communication.

FIGS. 3A-B provide illustrations of console devices 300a, 300b in accordance with embodiments of the invention. Console devices 300a, 300b are configured to supply potential energy in the form of high-pressure gas, e.g., from tank 321b. Connectors 310a, 310b are configured for connecting console devices 300a, 300b to a high-pressure gas source 321b. Input/output connectors and controls 320a may be used to supply regulated high-pressure gas onwards to other aspects of the system (e.g., an atherectomy subsystem and a pulsatile intravascular lithotripsy subsystem, in each case via a handle) and may include console input/output ports, which in some cases may comprise an HDMI port or a USB port for transmitting data to and or from consoles 300a, 300b. Display 330a may be used to provide real-time information to a user or operator, such as a clinician, such as pressure and flow rate readings, treatment duration, and other relevant information. Console 300a includes console inputs 340a for input to a controller present within console 300a for adjusting behavior of the system. Inputs 340a include buttons or controls for selecting a treatment intensity and mode as well as an emergency shut-off button for enabling and disabling the console assembly 300a. Consoles 300a, 300b also include a console encasement 390a, 390b configured to form a housing e.g., configured to protect the console components, e.g., during an accidental fall or during packaging. Console encasements 390a, 390b, when present, may be fabricated from a suitably rigid material, e.g., polymeric material, and may be transparent or opaque, as desired. Console 300b further comprises mechanical regulator 322b1 and electronic regulator 322b2 for adjusting potential energy received from pressure source 321b via inlet 310b prior to being output at handle connection 320b.

As reviewed, systems of the present invention include a console. The console, also referred to as console unit or console subsystem or console assembly, is used in embodiments of systems according to the present invention to generate the required power and control for treatment of biological tissue, such as cardiovascular tissue, using the system.

Embodiments of consoles according to the present invention include, or are operably connected to, a potential source. Potential sources of embodiments of the invention are configured to provide energy, which may be regulated as desired by a regulator. Any convenient potential source may be employed, where examples of potential sources include voltage sources, pressure sources, electromagnetic sources, electric field sources, chemical sources, and the like. In some embodiments, the potential source is a pressure source, where examples of suitable pressure sources include, but are not limited to: compressed gas cylinders, compressors and the like. Where desired, the potential source may be operably coupled to a regulator, which serves to modulate energy from the potential source to a suitable form so that it may be further acted upon, e.g., by an oscillator or other aspects of a manifold assembly. For example, where the potential source is a high-pressure gas source, the regulator may serve to regulate the pressure of the gas to a suitable value that can be input to an oscillator. In addition to positive potential sources (e.g., high-pressure gas), potential sources of interest may also include a negative potential compared with a reference or standard potential, e.g., a potential source configured to provide a vacuum potential compared to standard atmospheric conditions.

In some embodiments, the console comprises more than one potential source. In embodiments that comprise more than one potential source, the potential energy supplied by each potential source may all be of the same type or may be a combination of different potential types. For example, each potential source may be a pressure source (at the same or different potential levels), or, alternatively, one potential source may be a pressure source and another potential source may be a voltage source.

In embodiments, console assemblies may further comprise one or more regulators (i.e., power regulators), an output port and a controller. With respect to power regulators, as described above, in embodiments, the potential of the potential source may be regulated from a first, input potential, to a second potential, e.g., a potential that is suitable for transmitting to an oscillator or another aspect of a manifold assembly or the atherectomy subsystem or the pulsatile intravascular lithotripsy subsystem, ultimately for treatment of biological tissue, such as cardiovascular tissue. The potential of the potential source may be regulated to a pre-determined value, a user-set value or may be adjusted according to a variety of feedback inputs that occur during treatment. In some cases, the potential of the potential source may be dynamically regulated based at least in part on conditions related to treatment involving imparting pulsatile energy to biological tissue, such as cardiovascular tissue, e.g., based on changes in tissue compliance during treatment. In some cases, the potential of the potential source may be regulated in real time or substantially in real time. In certain embodiments, the potential of the potential source may be adjusted to an optimal value for a certain treatment. For example, the potential of the potential source may be adjusted to an optimal value for treatment of diseased heart tissue, such as heart tissue with calcifications or an occlusion, or, for example, the potential of the potential source may be adjusted to an optimal value for treatment of diseased heart tissue utilizing atherectomy subsystem versus the pulsatile intravascular lithotripsy subsystem. In some cases, one or more inputs from one or more of the console, the handle, the atherectomy subsystem, the pulsatile intravascular lithotripsy subsystem, a user or an oscillator, or from a source external to the system (e.g., other measurements regarding a subject, such as imaging of the subject) may be used to determine an optimal treatment condition, e.g., output potential of the potential source appropriate for the desired treatment (such as treatment utilizing the atherectomy subsystem versus treatment utilizing the pulsatile intravascular lithotripsy subsystem of the combined system), and then to adjust to that condition.

In embodiments that comprise a regulator (i.e., a power regulator or potential regulator) configured to regulate the potential of the potential source, such a regulator may be a passive (i.e., preset or user-adjusted regulator) or an active regulator (i.e., a regulator that is controlled with, for example, an electrical impulse or other dynamic signal from, e.g., a controller). Regulators of interest may comprise regulators typically used for fluidic regulation such as a directional or diaphragm valve, electrical regulation such as a voltage regulator, optical power regulation or the like. In embodiments that comprise more than one potential source, the potentials of the various potential sources may be regulated together or separately.

In embodiments, the regulated and/or unregulated potential (i.e., potential energy) from the potential source is output through an output port operably coupled to a handle for transmission to an atherectomy subsystem and/or a pulsatile intravascular lithotripsy subsystem. Any convenient output port, such as commercially available connectors, such as pneumatic, hydraulic, electrical or optical connectors, may be employed in embodiments. In certain instances, the unregulated or regulated potential energy may be converted to another energy form prior to or, in some cases, after the energy is output from the console, i.e., passed or otherwise transmitted to other aspects of the system.

In some cases, the console may include more than one physically separate or connected units, i.e., each, a console unit, that may be operably interconnected (e.g., electrically, fluidically, using radio frequency (RF) or the like). That is, the console may comprise a unitary assembly or two or more distinct, operably connected units.

In some instances, at least some of the console components are present in a unit that is configured to be hand-held or manipulated, e.g., moved, by hand. While the form factor of such a unit may vary as desired, in some instances, such units may be configured substantially as a rectangular box having height ranging from 10 to 100 cm, such 20 cm to 30 cm, width from 5 to 100 cm, such as 10 to 20 cm and depth ranging from 10 to 100 cm, such as 20 to 30 cm, and a mass ranging from 1 to 20 kg, such as 5 to 8 kg.

In an embodiment, a console may include a first console component that houses a potential source and regulator and actuator for the pressure source, e.g., a manipulatable button. The console may include an electrical connector for providing electrical connection to various other components of the system, as desired. For example, an electrical connector may be used to receive data regarding a position and/or configuration of a distal balloon or an atherectomy tool, such as a location or orientation vis-à-vis a tissue undergoing treatment, e.g., cardiovascular tissue, or lesion, e.g., a chronic total occlusion, or pressure or volume measurements, and to provide power to sensors configured to collect such data regarding treatment using the system.

In some instances, at least some of the console components are present in a mountable unit that is configured to be positioned or fixed on or proximal to an operating table near a subject, i.e., a patient, so that an operator, e.g., a physician, does not need to physically interact with the console assembly (for example, the operator does not need to be physically present in an operating room or a procedure room and can communicate with the system via a remote control at a distance) to treat the subject. In such instances, the mountable unit is designed to be easily clamped, fixed, or independently stable on, or proximal to, the operating table and can be operated by a distal control unit. In such instances, the mountable unit may include a communicator that provides for communication between the console assembly and the distal control unit, which may be implemented by any desired hardware and/or software configuration and may be configured to communicate using wired or wireless protocols.

Consoles and/or potential sources thereof that are employed in systems of the invention may be configured to be reusable or single use, as desired. Console assemblies employed in systems of the invention may be configured to receive a sterile sleeve such that the console assembly may be used while not contaminating a sterile field of the operating room. Further details regarding aspects of console units, potential sources, regulators, etc., that may be employed in embodiments of the present invention are provided in U.S. Pat. No. 11,464,949, United States Published Patent Application Publication No. 20200046949 as well as pending PCT Application Serial No. PCT/US2020/055458 as well as U.S. Application No. 63/274,832; the disclosures of which are herein incorporated by reference.

### Controller:

Embodiments of consoles of systems according to the present invention include one or more controllers also referred to as a controller or control assembly or control subsystem. Embodiments of systems may utilize a first logical controller operably connected to the atherectomy subsystem and a second logical controller operably connected to the pulsatile intravascular lithotripsy subsystem. In embodiments, such first and second controllers may be referred to as the controller. In embodiments such first and second controllers control, among other things, the amount and duration of energy transmitted to tissue, such as, for example, cardiovascular tissue, by each of the atherectomy subsystem and the pulsatile intravascular lithotripsy subsystem.

In some instances, embodiments of systems may utilize a controller to measure the effect of treatment on cardiovascular tissue, such as an extent to which an atherectomy tool has penetrated a lesion comprising calcified tissue or a degree of disruption of a lesion comprising calcified tissue. For example, controller may be configured to measure an extent to which a treatment has caused changes in cardiovascular tissue compliance. In still other instances, embodiments of systems may utilize a controller to control the distal and proximal movement of an atherectomy tool as it pecks into an occlusion.

In embodiments, the controller may be connected to, receive information from, and/or adjust or control aspects of one or more of the system, including the console thereof, such as a pressure source or a regulator thereof, the handle, an oscillator, the atherectomy subsystem or the pulsatile intravascular lithotripsy subsystem. The controller may also be configured to receive information from, and/or control, external systems such as an electrocardiogram (ECG), an intravascular or external pressure monitor, a blood volume sensor, patient vitals sensors or an imaging system such as an imaging system utilizing fluoroscopy, intravascular ultrasound (IVUS) or optical coherence tomography (OCT). Further, the controller may comprise multiple control units, e.g., the first and second controllers described above, interconnected such that one or more of the units synchronize and communicate with each other.

In some cases, the controller, or control units that, together, comprise the controller, may be configured to communicate with components of the system such that energy transmitted via the atherectomy subsystem, including via the atherectomy tool, or via the pulsatile intravascular lithotripsy subsystem, including via the distal balloon, is, in each case, appropriate, i.e., appropriate for a particular treatment involving utilizing an atherectomy tool and/or applying pulsatile energy to tissue. In other embodiments, the controller may receive information, e.g., data signals, from sensors regarding the status of a cardiovascular tissue treatment, such as, for example, an atherectomy treatment where sensors provide information regarding location, such as, for example, location of the atherectomy tool relative to an occlusion, a degree to which a hole is drilled through an occlusion, a degree to which a hole in a lesion or occlusion is expanded, and the like.

In embodiments, a controller may be configured to provide feedback to an operator of a system of the present invention in any convenient manner. In some cases, the controller is configured to provide tactile feedback to an operator by, for example, vibrating. For example, the controller may be configured to cause the handle to vibrate or to cause another interface to vibrate upon a relevant change or determination, such as measurement of a sensor, for example, the piercing of a hole through an occlusion or relevant changes in compliance of the cardiovascular tissue. Such tactile feedback may be used in connection with indicating to an operator of an embodiment of a system to change a configuration of the system.

### Manifold Assembly, Oscillator or Switch:

Embodiments of systems of the present invention include a manifold assembly. The manifold assembly, also referred to as manifold unit or manifold subsystem, may be used in embodiments of systems according to the present invention to receive energy transmitted from the potential source of the console and to transmit such energy to the atherectomy subsystem and to the pulsatile intravascular lithotripsy subsystem. In embodiments, the manifold assembly comprises an oscillator configured to generate pulse energy from energy transmitted from the potential source. In such instances, the oscillator is used to modulate a magnitude and/or timing and/or frequency of the potential energy from the potential energy source in order to provide for the desired energy for use in applying pulsatile energy to biological tissue, such as cardiovascular tissue, via an atherectomy tool and/or a distal balloon. Pulsatile energy may be utilized by atherectomy subsystem in connection with, e.g., rotating or orbiting, an atherectomy tool, or, in some cases, in connection with moving the atherectomy tool in a distal and a proximal direction in order that the atherectomy tool pecks into and out of a lesion such as a chronic total occlusion. In embodiments, a manifold assembly may be disposed within a handle, where such handle comprises an oscillator, as described herein, and is configured for manual manipulation by an operator.

In embodiments, the manifold subsystem includes an input connection operably connected to an output of the console, one or more oscillators and an output connection for use in connection with ultimately transmitting energy to the atherectomy subsystem and/or the pulsatile intravascular lithotripsy subsystem. Embodiments of handles may be configured for use with each of an atherectomy subsystem and/or a pulsatile intravascular lithotripsy subsystem, or may be configured with separate outputs for each such subsystem. In some embodiments where the console comprises one or more console subunits, as described herein, the manifold input connection comprises an input connection to one or more of the console subunits of the console. As described above, in embodiments, the manifold subsystem is configured to receive energy transmitted from the console and output energy, ultimately, to the atherectomy subsystem and the pulsatile intravascular lithotripsy subsystem. The manifold subsystem may be configured to receive various forms of potential energy from the one or more consoles (e.g., a voltage potential, an electromagnetic potential, a pressure potential or the like) and to distribute that energy to one or more oscillators in the manifold assembly. Any convenient input and output connections, such as commercially available connectors, such as pneumatic, hydraulic, electrical or optical connectors, may be employed in embodiments.

In certain instances, the manifold assembly or handle may receive potential energy such as from one or more console subunits of the console and distribute that potential energy to one or more oscillators in the manifold assembly or handle. In some cases, there is a one-to-one correspondence between console subunits and oscillators in the manifold subsystem or handle. In other cases, a single console subunit may deliver energy to one or more oscillators. In still other cases, one or more console subunits may deliver energy to a single oscillator; i.e., such that the potential energies of the one or more console subunits are combined in a single oscillator.

In embodiments, energy transmitted to an oscillator comprises a regulated or unregulated fluid under pressure. The oscillator may be actuated to output a pulsatile and/or a static pressure output. In certain embodiments where the potential energy transmitted by the console is a regulated or unregulated fluid under pressure, the oscillator may comprise a solenoid valve. Such solenoid valve may comprise, for example, a two-position, three-way, normally closed solenoid valve. In such instances, the solenoid valve is configured to receive the high-pressure regulated or unregulated fluid. Such a solenoid valve may be configured to have two modes, an “on” mode and an “off” mode. Such a solenoid valve may be configured to have three ports: a port operably connected to the high-pressure regulated or unregulated fluid (i.e., an input port), a port operably connected to, ultimately, a catheter assembly (i.e., a first output port), and an exhaust port (i.e., a second output port). The solenoid valve may be configured such that when turned on (i.e., in an “on” mode), the valve allows the high-pressure regulated or unregulated fluid to be transmitted, i.e., transmitted downstream in the system, such as transmitted to the atherectomy subsystem and/or the pulsatile intravascular lithotripsy subsystem. The valve may be further configured such that when turned off (i.e., in an “off” mode), the solenoid changes, i.e., reverses, the connected ports such that the distal side of the valve is exhausted (e.g., exhausted to atmosphere or vacuum). That is, in the “off” mode, the first output port may be connected to the second output port, thereby exhausting high pressure fluid present on the distal side of the solenoid valve.

In certain embodiments, a frequency and/or duty cycle of the oscillator may be adjusted to generate the appropriate output for treatment and for the atherectomy subsystem, including an atherectomy tool thereof, or for the pulsatile intravascular lithotripsy subsystem, including a distal balloon thereof. In various embodiments, the one or more oscillators may be configured to oscillate at one or more frequencies and/or duty cycles. In certain instances, an oscillator is configured to oscillate at a frequency between 0 and 50 Hz, such as 1-10 Hz or 10-20 Hz or 21-30 Hz or 31-40 Hz or 41-50 Hz, and a duty cycle between 10% and 90%, such as 10% or 20% or 30% or 40% or 50% or 60% or 70% or 80% or 90%. In other instances, such as those in which an oscillator is configured to use fluid pressure to deliver pulsatile pressure pulses for a treatment involving applying the atherectomy subsystem to cardiovascular tissue, the oscillator may be configured to or controlled to oscillate at a frequency between 0.25 Hz and 5 Hz, such as 1 Hz or 2 Hz or 3 Hz or 4 Hz or 5 Hz, and a duty cycle between 10 and 90%, such as 10% or 20% or 30% or 40% or 50% or 60% or 70% or 80% or 90%. In instances in which an oscillator is configured to deliver pulse energy comprising an optical or high voltage source, the oscillator may be oscillated at a frequency of 0.1 Hz to 1 GHz such as 1 Hz or 2 Hz or 3 Hz or 4 Hz or 5 Hz or more and a duty cycle between 0.0001% and 90% such as 0.001% or 0.01% or 0.1% or 1% or 10% or 20% or 30% or 40% or 50% or 60% or 70% or 80% or 90%.

Further details regarding aspects of manifold assemblies, handles, oscillators, etc., and components thereof, that may be employed in embodiments of the present invention are provided in U.S. Pat. No. 11,464,949, United States Published Patent Application Publication No. 20200046949 as well as pending PCT Application Serial No. PCT/US2020/055458 as well as U.S. Application No. 63/274,832 as well as U.S. Application No. 63/545,060; the disclosures of which are herein incorporated by reference.

In certain embodiments, output from the oscillator, or, in embodiments with more than one oscillator, outputs from the various oscillators, can be transmitted to one or more locations. In other embodiments that comprise more than one oscillator, the oscillators can be synchronized with each other, e.g., such that pulsatile energy transmitted from each oscillator is synchronized as desired, e.g., in terms of magnitude, frequency, phase, duty cycle, etc. In other embodiments with one or more oscillator, the oscillators can be synchronized with external factors or systems or sensors such as, for example, the results of an electrocardiogram (ECG), or can be adjusted based on feedback from the controller or other subsystems (e.g., such as volume or pressure measurements that originate from the atherectomy subsystem or the pulsatile intravascular lithotripsy subsystem). In other embodiments, the one or more oscillators may be controlled based on data regarding the location of the atherectomy tool, i.e., relative to an occlusion, e.g., data related to whether the atherectomy tool has pierced a lesion, such as a chronic total occlusion. That is, a controller may be configured to adjust the behavior of an oscillator based at least in part on such data.

In embodiments, the manifold assembly or handle may further comprise multiple inlet sources (e.g., connections to consoles), a housing (e.g., a manifold or handle encasement for the manifold assembly or handle), a multitude of oscillators, oscillator connection points (e.g., for transmitting energy from the oscillatory to a catheter assembly), controller connection points (e.g., for transmitting input from sensors within and/or external to the system) and a user feedback and/or control area (e.g., for a user to adjust the operation of the system). In embodiments, a manifold assembly may be disposed entirely or partially within a handle of the system, as described herein. In other embodiments, the manifold assembly may be disposed entirely or partially within the console of the system, as described herein.

In various embodiments, the manifold assembly or handle, like other aspects of systems of the invention, may be configured to be disposable or reusable. In cases where the manifold assembly or handle assembly is reusable and could contact a patient area, such assembly can be configured to be covered in a disposable, sterile sleeve or bag. In certain embodiments, the manifold assembly can be configured as a part of the console (i.e., such that components of the console and manifold assembly are located within a single common housing). In other embodiments, the manifold assembly can be configured in the form of a handle, i.e., the handle of an embodiment of the system, such that an operator of the system may hold the manifold assembly during use or therapy.

In some embodiments, components of the control subsystem or controller, as described above, can be located within the manifold assembly housing and/or within the console housing and/or within the handle. In certain cases, the manifold assembly, and/or the console and/or handle, includes a user interface configured such that an operator of the system has access to the manifold assembly or the console to start or stop treatment, adjust treatment intensities, adjust treatment modes or adjust other relevant aspects or configurations of the system.

### Handle and Tether:

Embodiments of systems of the invention also include a handle, which may be configured to operably connect with a console, e.g., as described above, via a tether. As described herein, in some embodiments, handles or handle assemblies comprise a manifold assembly, as described above, and/or oscillator. In some instances, the handle, e.g., a tether thereof, includes connectors, e.g., for connecting to the console device. The connectors may be designed to be easy to connect and disconnect, ensuring a secure and reliable connection between the console and the handle. The connectors may also be designed to provide feedback to the user, indicating a proper connection. This feedback may include an audible or visual signal, such as a click or a light indicator, or a tactile signal such as a vibration, which indicates that the connectors are properly mated, and the system is ready for use. Any convenient connectors, such as commercially available connectors, such as pneumatic, hydraulic, electrical or optical connectors, may be employed in embodiments. In other embodiments, the handle may be operably connected to, or may comprise a manifold assembly or aspects of a manifold assembly, as described herein.

In embodiments where the console includes a pneumatic system, the pneumatic system of the console may be designed to maintain pressure without leakage, ensuring the safe and effective delivery of the regulated gas. This may be achieved through the use of O-ring seals, compression fittings, or other pneumatic sealing methods. The pneumatic system may be designed to be reliable and efficient, ensuring that the regulated gas is delivered consistently and without interruption.

The electrical connector of the console may be designed to provide power and communication between the console and the handle, e.g., via a tether thereof (i.e., such that the handle may be powered by, and in communication with, the console via such electrical connector). This may be achieved through the use of a wired connection, such as a USB or RS232 cable, or a wireless connection, such as Wi-Fi or Bluetooth. The electrical connector may be designed to provide reliable and efficient power and communication, ensuring that the console and the handle, e.g., via a tether thereof, can communicate effectively and that the console and/or the handle and/or a manifold assembly, when present, can receive power as needed.

The console device and/or handle may be designed to detect when one or more connectors disconnect from each other or otherwise comprise an open circuit. This may be achieved through the use of a physical interlock mechanism, a pressure sensor, or other techniques. If the connectors become disconnected, the console and/or the handle may be configured such that the console immediately stops the delivery of energy, such as the delivery of regulated gas, and provides a visual or audible alarm to the user, i.e., operator, indicating that the connection has been lost. In such instances, the console and/or handle may be configured to also record the disconnection event and provide information to the user or an external device through the electrical connector.

In embodiments, aspects of a handle, e.g., a tether thereof, is configured to connect the console device to the handle. The tether may be designed to be flexible, durable, and reliable, allowing for easy maneuverability of the handle while maintaining a secure connection between the console and the handle, as well as components operably connected to the handle. While the tether may vary, in some instances the tether ranges from 10 inches to 20 feet long, allowing for a wide range of movement and positioning of the handle. In embodiments, the tether is typically 10 inches to 20 feet long, providing a wide range of movement and positioning for the handle, and therefore a wide range of movement and orientation available for the atherectomy subsystem and the pulsatile intravascular lithotripsy subsystem.

In embodiments, the tether includes two pneumatic lines, including a high-pressure inlet hose and an exhaust hose, as well as a bend-and-stay wire and an electrical cable. The pneumatic hoses may be configured to carry or transmit regulated gas from the console to the handle, while the bend-and-stay wire may be configured to provide stability and support to the tether. The electrical cable provides power and communication, i.e., communication of data and/or control signals, between the console and the handle.

The tether may be assembled using methods that ensure a secure and reliable connection between the various components. This may include the use of crimping, welding, or other methods to secure the pneumatic hoses and electrical cable in place. The tether may also be covered with a protective layer, such as a polyurethane or silicone coating, to protect against wear and tear and to ensure that the tether remains flexible and durable.

In embodiments, the handle may be configured to receive the tether components, including the high-pressure inlet hose, exhaust hose, bend-and-stay wire, and electrical cable. The handle may have several important functions, including controlling the gas flow output and providing a physical connection point between the console and the medical device, i.e., the atherectomy subsystem, e.g., the atherectomy tool, and/or the pulsatile intravascular lithotripsy subsystem, e.g., a pulsatile intravascular lithotripsy catheter and/or a distal balloon. The handle may be designed with a front (i.e., distal) connector configured to physically receive a medical device connector (i.e., a connector connecting the handle to the atherectomy subsystem and/or the pulsatile intravascular lithotripsy subsystem), allowing the system to regulate the gas, electrical power, and communication delivered to the medical device (i.e., the atherectomy subsystem and/or the pulsatile intravascular lithotripsy subsystem).

Where desired, an embodiment of a handle may include one or more user interface features, such as a button for user feedback, which allows the user (i.e., the operator) to provide feedback on the status of the system. This can be used to initiate or end a procedure, for example, or to adjust the intensity or duration of the treatment or to adjust the type of treatment applied, i.e., treatment via the atherectomy subsystem versus the pulsatile intravascular lithotripsy subsystem. Additionally, the handle may include LEDs, e.g., to provide the user with feedback, allowing an operator to quickly and easily determine the status of the system.

The handle may include a pneumatic outlet that provides regulated gas to the medical device (i.e., the atherectomy subsystem and/or the pulsatile intravascular lithotripsy subsystem). It may also have an electrical connection to the medical device connector, allowing for communication between the console and the medical device. The handle may be designed with a strain relief on the backend (i.e., proximal end) to ensure that the connections remain secure, even during movement or handling or otherwise manipulating the handle during treatment.

In addition to the physical components, the handle may also comprise an electronic assembly (i.e., an electronic board, such as a printed circuit board) to control the procedure and receive feedback. This electronic assembly can run software programs that determine the treatment type, intensity, and duration, and can be preset, user-adjusted, based on sensor input, or based on artificial intelligence. The handle can be configured in either a handheld or tabletop configuration, and the length of the tether, e.g., as described above, can in some instances vary between 10 inches and 20 feet, depending on the specific needs of the medical facility.

In embodiments, the handle is operably connected to the atherectomy subsystem and the pulsatile intravascular lithotripsy subsystem. In some cases, the handle comprises a single unit configured to house a switch, i.e., an oscillator, or other aspects of a manifold assembly described above and is configured to transmit energy to the intravascular pulse lithotripsy subsystem and atherectomy subsystem. In embodiments, the handle is configured to readily be connected and disconnected from each of a pulsatile intravascular lithotripsy subsystem and an atherectomy subsystem. That is, embodiments, a user of a system may first connect an atherectomy subsystem to the handle, perform an atherectomy procedure, and subsequently disconnect the atherectomy subsystem and connect a pulsatile intravascular lithotripsy subsystem and perform pulsatile intravascular lithotripsy; i.e., such that a single handle (and console and potential source) may be used with each of an atherectomy subsystem and a pulsatile intravascular lithotripsy subsystem.

In embodiments, the handle is configured to be held by an operator, e.g., during use. Handles of interest comprise any convenient shape, such as substantially cylindrical or substantially rectangular, and may, for example, have a height ranging from 10 to 100 cm, such 20 cm to 30 cm, a width from 5 to 100 cm, such as 10 to 20 cm and a depth ranging from 10 to 100 cm, such as 20 to 30 cm, and a mass ranging from 1 to 20 kg, such as 5 to 8 kg. In embodiments, the handle comprises one or more tactile features, i.e., to facilitate holding the handle by hand, e.g., by a gloved hand. Such tactile features may comprise groves or indentations.

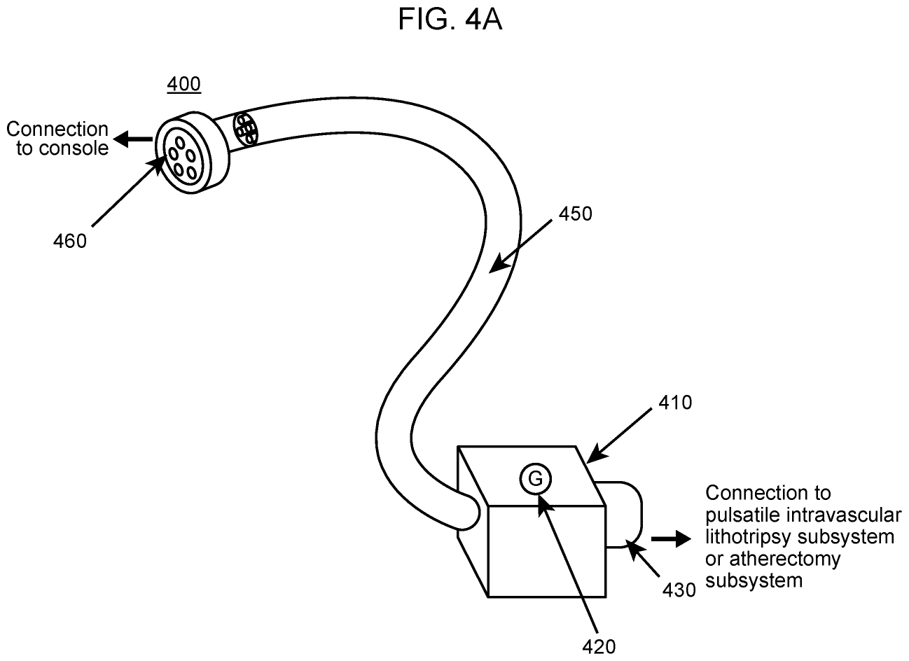

FIG. 4A presents a schematic of a handle 400 with a tether 450 according to aspects of the present invention. Handle tether assembly 450 comprises hand-held body 410 and tether 450. Hand-held body 410 is present at a relatively distal region of handle 400. Handle 400 includes button 420 for receiving user feedback. Button 420 further comprises LEDs for providing feedback to a user, i.e., an operator. Output connector 430 of hand-held body 410 of handle 400 is located at a relatively distal end of handle 400 and is configured to interface with each of an atherectomy subsystem and a pulsatile intravascular lithotripsy subsystem; that is, regulated, pressurized fluid, e.g., gas, is transmitted through output connector 430 of handle 400 to provide energy to each of an atherectomy subsystem and a pulsatile intravascular lithotripsy subsystem, when each such subsystem is connected to handle 400. Input connector 460 is present at a relatively proximal end of tether 450 of handle 400 and is configured to interface with a console. That is, input connector 460 is configured to receive pressurized fluid, e.g., gas, as well as power, e.g., electrical power, from the console via input connector 460, and also comprises an exhaust connection. Input connector 460 is further configured to receive and transmit electrical signals comprising data and/or control signals.

As described, embodiments of handles according to the invention are configured to be releasably engaged to embodiments of a connector of an atherectomy subsystem and a connector of a pulsatile intravascular lithotripsy subsystem, such that either such connector can be operably connected to the handle. FIG. 4B shows handle assembly 400 as well as a connector 401 in accordance with an embodiment of the invention. Connector 401 may be an atherectomy connector (such as, e.g., connector 113 of system atherectomy subsystem 160 or connector 213 of atherectomy subsystem 260) or a proximal connector (such as, e.g., proximal connector 151 of pulsatile intravascular subsystem 150 or proximal connector 251 of pulsatile intravascular subsystem 250). In each case, a connector of the atherectomy subsystem and a connector of the pulsatile intravascular lithotripsy subsystem comprise identical proximal interfaces for interfacing with a distal interface of the handle. Connector and handle interfaces may comprise, for example, complementary shapes, operable connections (e.g., high pressure fluid connections), electrical connectors, other interlock mechanisms, etc., and such aspects may be identical between an atherectomy connector of an atherectomy subsystem and a proximal connector of a pulsatile intravascular lithotripsy subsystem. FIG. 4B illustrates the general orientation of how connector 401 may be operably connected to handle 400, i.e., such that a proximal interface of connector 401 may be brought into contact with a distal interface of handle 400, such that the handle and connector are releasably engaged to form an operable connection therebetween, and, further, such that each of an atherectomy subsystem and a pulsatile intravascular lithotripsy subsystem can be interchangeable operably connectable to the handle. FIG. 4B further illustrates how the shape, alignment features, latching features and connectors of handle 400 and connector 401 align when such two elements are brought into contact with each other, i.e., operably connected to each other. Further details regarding interfaces between embodiments of handles and connectors are provided in U.S. Application No. 63/545,060.

### Atherectomy Subsystems:

The atherectomy component (i.e., an atherectomy subsystem) is a catheter-based system configured to intravascularly remove plaque buildup. The atherectomy component may be configured to perform any type of suitable atherectomy procedures. Atherectomy procedures of interest that may be implemented by embodiments of systems of the invention may include, but are not limited to: excisional atherectomy, laser ablation atherectomy, orbital atherectomy and rotational atherectomy, or otherwise utilize ultrasound, electrohydraulic lithotripsy (EHL) cavitation emitters and/or mechanotransduction of a guidewire to create a bore through a plaque buildup, e.g., a chronic total occlusion.

In some instances, the atherectomy tool includes a rotational device, e.g., a rotational assembly, that converts the output of the oscillator (e.g., voltage, current, pressure, or flow) provided by the handle into rotational motion. This rotational motion is transferred to a flexible drive shaft, e.g., a lateral transmission assembly, that has both a proximal and distal end. A portion of the proximal end is attached to the rotational motor, and a portion of the distal end has a grinding burr. When the motor is rotated, the motor transfers its rotational motion through the drive shaft and to the attached grinding burr. For stability, the drive shaft and grinding burr can have a guidewire which passes through it. Further, there can be an advancer, which advances or retracts the grinding burr through the lesion. The atherectomy portion of the system can have a number of sensors including current-sensing, rotational position, speed, and/or acceleration sensing, heat and/or temperature sensing, linear position sensing, torque sensing, pressure and/or flow sensing. In other instances, the atherectomy tool includes a rotational device, e.g., a rotational assembly, that converts the output of the switch (e.g., voltage, current, pressure, or flow), e.g., provided by the handle and ultimately from the console, into orbital motion, i.e., such that an atherectomy tool orbits to grind the tool through the lesion.

Rotational or orbital atherectomy tools/subsystems that may be components of systems of the invention include, but are not limited to, those described in U.S. Pat. Nos. 11,559,324; 11,478,270; 11,413,063; 11,382,652; 11,331,119; 11,291,468; 11,172,956; 11,096,716; 11,090,079; 11,065,030; 10,893,882; 10,729,460; 10,441,311; 10,405,879; 10,405,878; 9,084,627; 9,554,823; and U.S. Published Patent Application Publication Nos. 20220387074; 20220240975; 20220142667; 20220133347; 20220133346; 20220061879; 20210322052; 20210251653; 20210145474; 20210077143; 20200397464; 20200397463; 20200315653; 20200229844; 20200222075; 20200214735; 20190365412; 20190343551; 20190307483; 20190262034; 20190262032; 201902602022; 20190201052; 20190201051; 20170135719; 2016157886; 20160022307; 20140277010; 20140005699; and 20130086588; the
disclosures of which are herein incorporated by reference.

In some embodiments, the atherectomy subsystem comprises an atherectomy tool. Such atherectomy tool may comprise a rotational atherectomy tool or an orbital atherectomy tool or one or more of a laser tool, an ultrasound tool, an electrohydraulic lithotripsy (EHL) cavitation emitter tool or a mechanotransduction tool. In embodiments, the atherectomy tool is present at a distal region of the system and may be configured to grind within a lesion. In some cases, the atherectomy tool is configured to cross a lesion, such as an occluded lesion or a chronic total occlusion (CTO). In other cases, the atherectomy tool is configured to create a new channel within an occlusion. For example, the atherectomy tool may be configured to drill a hole in an occlusion. As described, the atherectomy tool may be a burr, such as a grinding burr.

As described, atherectomy subsystems may comprise a rotational assembly configured to produce rotational energy, such as a rotational motor. In embodiments, the rotational assembly is operably connected to the console or to a potential source of the console, e.g., via the handle and a tether. In embodiments, the rotational assembly is configured to transduce energy transmitted from the console into rotational energy.

The atherectomy subsystem may further comprise a lateral transmission assembly configured to propagate rotational energy from the rotational assembly to the atherectomy tool. Such lateral transmission assembly may be positioned distal to the rotational assembly and proximal to the atherectomy tool. In embodiments, the lateral transmission assembly comprises a rotational drive shaft, such as a flexible drive shaft. The lateral transmission assembly may be configured to receive a guidewire, e.g., via a guidewire lumen. Such guidewire and guidewire lumen may comprise a fluid bearing, i.e., such that fluid is present within the guidewire lumen, of the atherectomy subsystem configured to dissipate heat. When present, the guidewire may be configured to impart stability to the lateral transmission assembly while propagating rotational energy from the rotational assembly to the atherectomy tool. When present, the guidewire may be further configured such that the atherectomy subsystem comprises an atherectomy tool configured to utilize mechanotransduction of the guidewire to bore through a lesion.

In certain cases, the atherectomy subsystem comprises an advancer configured to advance and retract the atherectomy tool in distal and proximal directions, e.g., towards and away from a lesion, such as a chronic total occlusion. An advancer may be configured to pulse the atherectomy tool into and out of an occluded lesion. Such advancer behavior may utilize pulse energy transmitted from the handle. In embodiments, the atherectomy subsystem further comprises a filter located distal to the atherectomy tool, such a filter configured to protect vasculature from distal emboli.

As described, embodiments of the atherectomy subsystem further comprise a sensor, where such sensor may be configured to sense one or more of current, rotational position, speed, acceleration, temperature, linear position, torque, pressure or flow. In embodiments, the sensor is configured to sense current associated with the atherectomy subsystem interfacing with an occluded lesion. That is, it may sense a current response associated with the atherectomy subsystem penetrating a lesion, such as, for example, an occlusion comprising calcified plaque.

### Pulsatile Intravascular Lithotripsy Subsystems:

Pulsatile intravascular lithotripsy subsystems of systems of the invention are configured to apply pulsatile energy to calcified tissue, such as calcified vascular tissue. In embodiments, these subsystems may include a proximal connector configured to operably connect the balloon catheter assembly to a pulse generator and to transduce a first pulse energy generated by the pulse generator to a second pulse energy. In embodiments, pulse generators comprise, as described herein, a potential source, a console and a handle, for example. Pulsatile intravascular lithotripsy subsystems further comprise a distal balloon and a catheter component, where the catheter component includes a fluidic passage operably positioned between the proximal connector and the distal balloon, which passage is configured to propagate the second pulse energy from the proximal connector along the fluid passage to the distal balloon.

As used herein, the frequency is the number of full pressure pulse cycles (peak-to-peak) per unit time; the duty cycle is the percentage of time allocated to the high-pressure segment of a single pressure cycle; and the amplitude is the difference between the maximum and minimum pressure. As the energy imparted by the balloon to the internal tissue is pulsatile, it changes (e.g., increases and decreases) at a defined or determined frequency and duty cycle. During pulsatile intravascular lithotripsy treatment, blood flow distal to the distal end balloon may be occluded, which may limit treatment time. To achieve a successful treatment within this time, the pulsatile frequency and amplitude must impart sufficient energy to the tissue to treat it. While the frequency of pulsatile energy imparted by the balloon to tissue associated therewith may vary, in some instances the frequency is high frequency, ranging in some instances from 0 to 100 Hz, such as 0 to 25 Hz. Similarly, the duty cycle of pulsatile energy imparted by the balloon to tissue may vary ranging in some instances from 10% to 100%, such as 60% to 80%. Amplitude of pulsatile energy imparted by the balloon to tissue may vary ranging in some instances from an internal balloon pressure from 0-100 ATM, such as 0-30 ATM. In some cases, during a given procedure, the frequency may vary over the course of the procedure, i.e., not remain constant, as desired.

Pulsatile energy, when applied to diseased luminal vascular tissue, is effective in treating diseased tissue, such as CP tissue, while reducing the negative effects on surrounding healthy tissue. Important characteristics of pulsatile energy for achieving successful treatments may include the frequency and amplitude of the delivered pulsatile energy. In embodiments, such pulsatile energy can enable the safe, controlled, fatigue fracture of CP-lesions. Fatigue fracture is the process of cyclically loading a structure below the pressure that yields instantaneous failure. Whereas conventional treatments apply dangerous high-pressure bursts to the vessel that may create dissections and perforations, pulsatile intravascular lithotripsy may employ lower pressure high-frequency oscillations in a balloon to initiate low-pressure fatigue fracture of CP-lesions.

The described embodiments are dynamic physical systems, in which the output of the system (e.g., actual frequency, duty cycle, and amplitude) is governed by the system input (e.g., desired frequency, duty cycle, and amplitude) and the system characteristics (e.g., catheter length, friction, and flow channel lumen diameter). Embodiments of the systems are configured for generating controlled mechanical lithotripsy pulses in an angioplasty balloon such that the system output tracks, in some instances with minimal attenuation, the commanded, or desired, input signal. Signal attenuation is the reduction in amplitude in the system output versus the input because of the characteristics of the physical system. For successful treatment, minimal attenuation, in that the output pulsatile energy remains substantially similar, e.g., in terms of frequency, duty cycle, and/or amplitude, to the input pulsatile energy as it propagates from the system input (e.g., proximal connector) to the system output (e.g., the distal balloon), is required. As such, in some instances any change in frequency, if present at all, between the proximal connector and distal balloon would be 30% or less, such as 5% or less. In some instances, any change in amplitude, if present at all, of the pulsatile energy between the proximal connector and distal balloon would be 30% or less, such as 5% or less. In some instances, any change in duty cycle, if present at all, of the pulsatile energy between the proximal connector and distal balloon would be 30% or less, such as 5%.

As summarized above, pulsatile intravascular lithotripsy subsystems, which represent mechanical systems, according to embodiments of the invention are operably connected to a pulse generator. In embodiments, pulse generators comprise, as described herein, a potential source, a console and a handle, for example. The pulse generator comprises a component which is configured to generate a first pulsatile energy that can be transduced by the proximal connector to a second pulsatile energy, e.g., as described in greater detail below. The proximal connector is configured to receive the first pulsatile energy provided by the pulse generator and transduce it to a second pulsatile energy that may be received by the distal balloon to impart pulsatile energy to an internal tissue location, as described herein, such as for use in DBA or pulsatile intravascular lithotripsy applications as well as for use in final post-dilatation of the vessel, for example in one treatment comprising both applications. That is, embodiments of the present invention may be used first to apply pressure pulses to luminal tissue, such as a vessel, to crack calcium (i.e., CP-affected tissue) and then to subsequently expand the vessel using traditional, non-compliant balloon post-dilatation. Embodiments of aspects of pulsatile intravascular lithotripsy subsystems are now reviewed in greater detail.

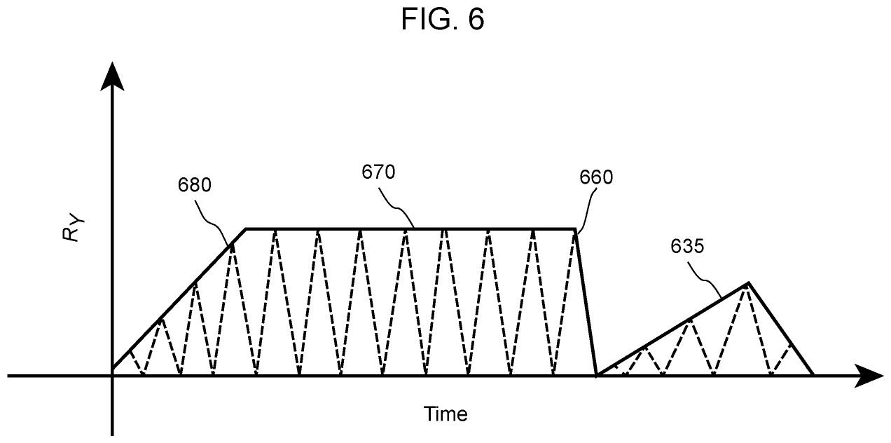

The first pulsatile energy may vary as desired, where examples of first pulsatile energy include, but are not limited to: pulsatile pressure energy, pulsatile mechanical energy, pulsatile electromagnetic energy, and the like. As the first pulsatile energy is pulsatile, the magnitude of the first energy changes or modulates over time, e.g., according to a determined or known, e.g., predetermined, frequency, according to the user, and/or according to treatment progression. While the frequency of the first pulsatile energy may vary, in some instances the frequency is high frequency, ranging in some instances from greater than 0 to 100 Hz, such as 2 to 25 Hz. As described below, during a given procedure, the frequency amplitude and/or duty cycle may vary over the course of the procedure, i.e., not remain constant, as desired (e.g., as described in conjunction with FIG. 6, below). The frequency, amplitude, and/or duty cycle also may vary depending on the type of catheter and distal balloon (e.g., balloon length and/or diameter, shaft length), treatment type, lesion stiffness, lesion density (e.g., as acquired by computed tomography or intravascular imaging), or lesion morphology etc. The frequency, amplitude, and/or duty cycle also may vary depending on user inputs, negative feedback from measurements, and/or positive feedback from system modeling.

As described herein, pulse generators of embodiments of the invention include potential sources configured to provide energy which may be regulated as desired by a regulator and an oscillator to provide for the pulsatile aspect of the first pulsatile energy. Any convenient potential source may be employed, where examples of potential sources include voltage sources, pressure sources, electromagnetic sources, electric field sources, chemical sources, laser sources and the like. In some embodiments, the potential source is a pressure source, where examples of suitable pressure sources include, but are not limited to: compressed gas cylinders, compressors, and the like. Where desired, the potential source may be operably coupled to a regulator, which serves to modulate energy to a suitable form so that it may be further acted upon by the oscillator. For example, where the potential source is a high-pressure gas source, e.g., as may be employed in a pneumatic pulse generator, the regulator may serve to regulate the pressure of the gas to a suitable value that can be input to the oscillator. In addition to the potential source and regulator, the pulse generators may include an oscillator. In such instances, the oscillator is used to modulate the magnitude and timing of the potential energy from the potential source to provide for the desired first pulsatile energy.

The disparate components of the pulse generator may be present in a single housing or provided as two or more distinct, operably connected units. In some instances, at least some of the pulse generator components are present in a unit that is configured to be hand-held, e.g., the handle. In such instances, the hand-held component, e.g., the handle, is designed to be held and operated in a single adult human hand. While the form factor of such hand-held units may vary as desired, in some instances, such units have a general diameter and/or width ranging from 20 to 150 mm, such as 50 to 80 mm and length ranging from 50 to 300 mm, such as 100 to 200 mm, and a mass ranging from 100 to 2000 g, such as 500 to 750 g. For example, a pulse generator may include a first console component, e.g., a console, as described herein, that houses, or is operably connected to, the potential source, and houses the regulator, and a second hand-held actuator, i.e., a handle as described herein, that includes the oscillator and an actuator for the oscillator, e.g., a manipulatable button. The hand-held actuator may include an electrical connector for providing electrical connection to various components of the pulsatile intravascular lithotripsy subsystem, as desired. For example, the electrical connector may be used to receive data regarding diaphragm position, memory, and/or pressure and to provide power to these sensors, where examples of such are further described herein.

In some instances, at least some of the pulse generator components are present in a mountable unit that is configured to be positioned or fixed on the operating table near a patient so that the physician does not need to be physically present to treat the patient. In such instances, the mountable unit is designed to be easily clamped, fixed, or independently stable on the operating table and can be operated by a distant control unit. In such instances, the mountable unit may include a communicator that provides for communication between the unit and the distal control unit, which may be implemented by any desired hardware and/or software configuration, and may be configured to communicate using wired or wireless (e.g., Bluetooth or radiofrequency) protocols. Pulse generators employed in systems of the invention may be configured to be reusable or single use, as desired. Pulse generators employed in systems of the invention may be configured to receive a sterile sleeve such that the generator may be used while not contaminating the sterile field of the operating room. Further details regarding pulse generators and components thereof, e.g., potential sources, oscillators, regulators, etc., that may be employed in embodiments of the present invention are provided in U.S. Pat. No. 11,464,949, United States Published Patent Application Publication No. 20200046949 as well as pending PCT Application Serial No. PCT/US2020/055458 and pending PCT Application Serial No. PCT/US2022/014785; the disclosures of which are herein incorporated by reference.

As summarized above, pulsatile intravascular lithotripsy subsystems of the invention include a proximal connector, a catheter and a distal balloon. The proximal connector is configured to receive first pulsatile energy from the pulse generator and transduce it to a second pulsatile energy that may be propagated along the length of the catheter, e.g., along the fluid, e.g., liquid, passageway thereof, to a distal balloon. As the proximal connector transduces the first pulsatile energy to a second pulsatile energy, it changes the form of the pulsatile energy in some way. Examples of changes to the form of energy that may be made by the proximal connector include, but are not limited to: gas pressure and/or flow to liquid pressure and/or flow, mechanical potential and/or kinetic energy to fluid pressure and/or flow, optical potential and/or kinetic energy to fluid pressure and/or flow, electric field potential and/or kinetic energy to fluid pressure and/or flow, magnetic potential and/or kinetic energy to fluid pressure and/or flow, and the like. For example, where the first pulsatile energy is a pneumatic first pulsatile energy, the proximal connector may be configured to transduce the pneumatic first pulsatile energy to a second hydraulic pulsatile energy that may be propagated from the proximal end of the pulsatile intravascular lithotripsy subsystem to the distal end, which is an example of gas to liquid transduction of the pulsatile energy. In some instances, the pulsatile intravascular lithotripsy subsystem propagates the second pulsatile energy from the proximal to distal end with little, if any attenuation, where the magnitude of any attenuation, if present, does not exceed 30% reduction, and in some instances does not exceed 5%, e.g., as described above.

In some instances, the pulsatile intravascular lithotripsy subsystem includes: (i) a proximal connector operably connecting the pulsatile intravascular lithotripsy subsystem to the pulse generator and configured to transduce a first pulse energy generated by the pulse generator to a second pulse energy; (ii) a distal balloon; and (iii) a catheter component that includes a fluidic passage operably positioned between the proximal connector and the distal balloon.

The proximal connector is a component of the assembly located proximally in the pulsatile intravascular lithotripsy subsystem, e.g., at the proximal end or near the proximal end, e.g., within 1 cm or closer to the proximal end, where the proximal connector is configured to operably connect the pulsatile intravascular lithotripsy subsystem to the pulse generator and transduce the first pulsatile energy to the second pulsatile energy, e.g., as described above. The manner in which the proximal connector operably connects to the pulse generator (i.e., the manner in which the proximal connector operably connects to a handle) may vary, as desired, where a given type of connector may be a press fit connector, latch connector, screw connector, threaded connector, magnetic connector, push-to-connect connector, Yor-lock connector, claw clamp connector, gasket connector, socket connector, flanged connector, cam-and-groove socket, quick-connect connector and the like, where aligners or detents may be employed, as desired, to provide for a connection that repeatably and accurately positions the proximal connector in relation to the pressure generator and/or electrical connectors. As described, such operable connection between the proximal connector and a handle is configured to be the same operable connection used to connect the connector of the atherectomy subsystem and the handle. For example, both the proximal connector and the connector of the atherectomy subsystem may comprise the same shape and the same latching mechanism, holding such connectors in place with respect to the handle. That is, systems of the invention utilize a uniform interface with respect to the atherectomy and pulsatile intravascular lithotripsy subsystems such that each subsystem may be interchangeably operably connected to the handle.

As reviewed above, in some instances the conversion is fluid to fluid energy conversion, e.g., where the first pulsatile energy is pneumatic pulsatile energy, and the second pulsatile energy is hydraulic pulsatile energy. In such instances, the proximal connector may include a proximal chamber and a distal chamber separated by a membrane, e.g., where the membrane hermetically seals the distal chamber from the proximal chamber. The proximal chamber may be configured to receive pneumatic pulsatile energy from the pulse generator. The volume of the proximal chamber may vary, ranging in some instances from 0.1 mL to 100 mL, such as 1 mL to 4 mL, where in some instances the proximal chamber is occupied by a gas. In certain instances, the proximal chamber forms a minimum volume chamber while still being large enough to accommodate the volume change required to fill a distal balloon. In this case, the time to fill this minimum volume chamber to a certain pressure is minimized, which allows the frequency of the pulsatile intravascular lithotripsy procedure to be increased. The distal chamber is fluidically coupled to the fluidic passageway of the catheter component. The volume of the distal chamber may vary, ranging in some instances from 0.1 mL to 100 mL, such as 1 mL to 4 mL, where in some instances the distal chamber is occupied by a liquid.

The membrane separating the proximal and distal chambers is configured to move in response to the first pulsatile energy and, in doing so, produces a second pulsatile energy in the distal chamber of the connector. The dimensions of the membrane may vary, where in some instances the membrane has an area ranging from 100 mm² to 5000 mm², such as 500 mm² to 2000 mm². The membrane may be fabricated from any convenient elastic (e.g., pliant) material, where in some instances the material has a hardness ranging from Shore 10 A to Shore 90 A, such as Shore 50 A, and a thickness between 0.5 mm to 5 mm, such as 1.0 mm to 2.5 mm. Examples of suitable membrane materials include, but are not limited to: silicone, rubber, and the like and in some cases may be strengthened by adding a reinforcing component, such as a braid. Where desired, a biasing component, such as a spring, may be provided to provide for a default or baseline membrane position. For example, a spring may be provided on the distal chamber side of the membrane which urges the membrane back to an initial position when force is removed from the proximal chamber side of the membrane. In other instances, the system may be controlled such that the pulse generator provides a constant pressure (albeit, small such as between 0.1 and 2 atm) to the proximal chamber to counteract the priming pressure. This counterforce would allow the diaphragm to be set to an appropriate position to start pulsatile intravascular lithotripsy treatment and to begin measurements.

While the form of proximal connectors of embodiments may vary, in some instances the proximal chamber is defined by a proximal flange and the distal chamber is defined by a distal flange, where the proximal and distal flanges are positioned on either side of the membrane to define the proximal and distal chambers, which may be sealed (e.g., hermetically) from each other by the separating membrane. In such instances, the proximal flange may include a proximal port normal to (e.g., axial to) the proximal flange configured to receive the first pulse energy, e.g., pneumatic pulsatile energy, generated by the pulse generator. While the dimensions of the proximal port may vary as desired, in some instances the port has an outer diameter ranging from 1 mm to 30 mm, such as 3 mm to 8 mm and inner diameter ranging from 1 mm to 30 mm, such as 2 mm to 7 mm. In such instances where the proximal flange has a proximal port the port may have a length ranging from 1 mm to 50 mm, such as 3 mm to 10 mm. In such instances, the distal flange may include a distal port fluidically coupling the distal chamber with the fluidic passage of the catheter. While the dimensions of the distal port may vary as desired, in some instances the port has a luminal diameter ranging from 0.1 mm to 10 mm, such as 1 mm to 3 mm.

In instances where the proximal chamber includes a proximal port, the proximal chamber is fluidically coupled to the port. In such instances, the junction between the proximal port and the proximal chamber may include a nozzle and/or diffuser, which, in some cases, may be formed geometrically by the proximal flange. In such instances, the nozzle or diffuser may act to increase or decrease velocity of the flow at the expense of fluid pressure. With such increase or decrease of velocity of the flow, characteristics of the energy conversion may be improved, such as ramp up time or smoothness of energy conversion. In cases of pneumatic flow, the speed of the gas may be high enough to induce compressible fluid phenomena such as in sonic or supersonic flows. In such cases, specialized flow nozzles such as a convergent-divergent nozzle may be used to optimize flow velocity.

Where desired, the proximal connector may include one or more sensors, e.g., configured to provide data regarding one or more components of the connector and/or the balloon catheter assembly. Any convenient type of sensor may be included in the proximal connector, where sensors of interest include, but are not limited to: pressure sensors, positional sensors, displacement sensors, proximity sensors, flow sensors, temperature sensors and the like. In some instances, the proximal connector includes a pressure sensor operably coupled to the distal chamber. In such instances, the pressure sensor may detect pressure and changes thereof in the liquid in the distal chamber. When included, any convenient type of pressure sensor may be present, where examples of pressure sensors that may be present include, but are not limited to: resistive, capacitive, piezoelectric, optical, and MEMS-based pressure sensors, and the like. In some cases, these pressure sensors may measure pressure at the proximal connectors and in other cases, the pressure may be read at or along the length of the catheter and/or distal balloon. To measure such pressures at the distal balloon, a fiber optic based sensor, for example, may be employed. In some instances, the proximal connector includes a membrane positional sensor configured to provide spatial data regarding the position of the membrane at a given time, e.g., during use of the subsystem. When present, any convenient membrane position sensor may be employed. In some instances, the membrane positional sensor is a Hall sensor, e.g., which may be employed in conjunction with one or more magnets (e.g., permanent or electromagnet) present at a fixed location relative to the membrane, such as a fixed location of the proximal connector such that the one or more fixed magnets are positioned to modulate voltage of the Hall Sensor upon membrane movement. In other instances, the membrane positional sensor may be an optical sensor, electric field potential sensor, resistive sensor, magnetic sensor, angle sensor, or acceleration sensor. Further, any combination of these sensors may be used to gather positional data of the membrane or diaphragm. In cases in which a combination of membrane positional sensors is employed, e.g., to ensure sensors provide correct data across a variety of frequencies, sensor data may be combined through “sensor fusion” techniques, such as those known in the art. When present, a membrane positional sensor may be employed for a variety of different purposes, e.g., to assess vessel compliance and treatment (such as described below), to assess proper filling of the proximal connector, catheter and/or distal balloon, to provide for a way to assess whether the membrane has been stretched beyond desired thresholds, etc. Fabrication methods of the membrane sensor may include, but are not limited to: adhesives, direct printing, welding, embedding and the like.

Where desired, the proximal connector may further include an electrical assembly. The electrical assembly may be configured to perform a number of functions, such as but not limited to, powering of one or more sensors, control of one or more sensors, storage of data obtained from one or more sensors, transmission of sensor data from one or more sensors to another location, storage of information about the balloon catheter assembly, writing and/or reading data and the like. The electrical assembly may vary, and in some instances may include circuitry and/or memory. When present, the memory may store a variety of different types of information, including but not limited to: information about the balloon catheter assembly and/or components thereof, e.g., the distal balloon, e.g., expiration date, batch number, balloon size (e.g., balloon diameter and length), balloon rated burst and nominal pressure, cycle limit (e.g., number of allowable cycles the balloon is rated for), and cycles used for, allowable pulse frequency or duration, previous use, balloon reference pressure-volume curve, and/or indication for use, etc. The electrical assembly, when present, may further include a connector, e.g., for operably connecting the electrical assembly to the pulse generator. The electrical assembly may be present in any convenient configuration, such as a printed circuit board, including a flexible printed circuit board. In some cases, the sensors may transmit data wirelessly, such as through Bluetooth RF.

The various components of the proximal connector, e.g., as described above, may be present in a housing or over-mold, e.g., configured to protect the proximal connector components, e.g., during an accidental fall or during packaging. The housing, if present, may be fabricated from a suitably rigid material, e.g., polymeric material, and may be transparent or opaque, as desired.

As summarized above, the pulsatile intravascular lithotripsy subsystem may include a catheter component positioned between the proximal connector and the distal balloon. The catheter component is configured to propagate or convey the second pulsatile energy from the proximal connector to the distal balloon, e.g., with minimal, if any, attenuation, such as described above. The catheter component includes a portion, e.g., a shaft, that is configured to be employed as a catheter, such that it may be introduced into a lumen of a human or another animal, e.g., mammal. While the dimensions of this portion may vary, in some instances this catheter portion has an outer diameter (OD) ranging from 1.50 mm to 2.50 mm, such as 1.75 mm to 2.20 mm.

While the structure of the catheter component may vary, in some instances the catheter component includes a proximal flexible tube; a distal catheter shaft (which is the catheter portion, e.g., as described above); and a connector connecting the distal end of the proximal flexible tube to the proximal end of the distal catheter shaft. The proximal flexible tube is made of a pliant material, e.g., braided or unbraided polyvinyl chloride (PVC), silicone, polycarbonate (PC), and the like, where the dimensions of the tube may vary. In some instances, the flexible tube has an inner lumen with a diameter ranging from 0.1 mm to 10 mm, such as 1 mm to 3 mm, and a wall thickness ranging from 0.1 mm to 5 mm, such as 0.5 mm to 2 mm. The length of the proximal flexible tube may also vary, ranging in some instances from 1 cm to 100 cm, such as 5 cm to 20 cm.

The distal catheter shaft may also vary. The distal catheter shaft may be fabricated from any suitable physiologically acceptable material, including but not limited to a polyimide, such as a polyimide braid, or a polyimide-type material and the like. In some instances, the distal catheter shaft has a length ranging from 10 cm to 5 m, such as 100 cm to 300 cm. The outer diameter of the distal catheter shaft may also vary, in some instances ranging from 1.50 to 2.50, such as 1.75 mm to 2.20 mm. The distal catheter shaft may include a first liquid passageway lumen, where the dimensions of this first liquid passageway lumen may vary. In some instances, the diameter of this first liquid passageway lumen ranges from 1.3 to 2.2 mm, such as 1.6 to 2.1 mm. The first liquid passageway may include one or more openings at the distal end for establishing liquid communication between the interior of the liquid passageway lumen and the interior of the distal balloon. The one or more openings, when present, are configured so as to not substantially attenuate, and in some instances, not attenuate at all, the second pulsatile energy as it enters the balloon from the liquid passageway. In some instances, these openings may be configured to be a nozzle and/or diffuser. In such instances, the nozzle or diffuser may act to increase or decrease velocity of the flow at the expense of fluid pressure. With such increase or decrease of velocity of the flow, characteristics of the balloon expansion may be altered, such as ramp up time, impact, force, and the like. The distal catheter shaft may also include a second guidewire lumen. When present, the dimensions of this second guidewire lumen may vary, where in some instances the diameter of the guidewire lumen ranges from 0.25 to 0.5 mm, such as 0.37 to 0.42 mm. The distal catheter shaft may be configured to traverse the entire length of the distal balloon or end at the proximal connection of the distal balloon. In the case where the distal catheter shaft spans the entire balloon length, the distal catheter shaft may be ported to enable fluid communication between the inside of the catheter shaft and the distal balloon. The porting may be performed using laser processes or other special machining processes. The pattern or distribution of ports in the catheter shaft may be arranged to ensure that the distal catheter shaft has the required stiffness to push through a narrow, calcified lesion and to prevent kinking but also the flexibility to traverse a long, tortuous lesion. Ported holes may be 0.05 to 1 mm in diameter such as 0.2 mm. The holes may be patterned in a helical or linear pattern or may follow the inner braid of the material. The number of holes can be between 100 and 500 such as 200. The total area of the holes should exceed the cross-sectional area of the flow channel lumen. By porting the entire length of the distal catheter shaft on which the distal balloon is present, the entire balloon surface receives an equal amount of pulsatile energy during treatment. This configuration ensures that, if a portion of the balloon is constricted, that portion as well as the other parts of the balloon receive an equivalent amount of energy. The drawback of this configuration is that the crossing profile (i.e., the overall diameter of the distal catheter shaft) is larger and is more difficult to pass through narrow lesions, or narrow volumes drilled in connection with an atherectomy procedure. To narrow the distal catheter shaft, the distal catheter shaft may end at the proximal balloon connection. In this instance, only the guidewire lumen traverses the length of the balloon. The balloon, in this case, may have a proximal neck diameter that matches the distal catheter shaft and a distal neck diameter that matches the guidewire lumen. Because the guidewire lumen has a smaller diameter than the distal catheter shaft, the crossing profile of this configuration may be improved, e.g., compared to the previously described configuration. However, there is only one port transferring fluid from the distal catheter shaft to the balloon, which can unevenly distribute energy to the walls of the balloon and the calcified lesion. These two configurations may be used in different instances such as for different indications, anatomical locations, etc.

Also present in the catheter component of these embodiments is a connector connecting the distal end of the proximal flexible tube to the proximal end of the distal catheter shaft. The connector may vary as desired. In some instances, the connector includes a first branch configured to provide guidewire access to a guidewire channel of the catheter shaft and a second branch configured to fluidically couple the lumen of the proximal flexible tube and liquid passageway lumen of the distal catheter shaft. An example of a suitable connector is a Y connector or a similar connector, in some embodiments, comprising a sufficient number of ports that a lateral transmission assembly of an atherectomy subsystem may be coupled to the catheter of the pulsatile intravascular lithotripsy subsystem. In such embodiments, the catheter of the pulsatile intravascular lithotripsy subsystem may comprise one or more channels, e.g., lumen, for housing aspects of a lateral transmission assembly of an atherectomy subsystem. For example, the catheter may comprise a channel or lumen that houses a guidewire operably connected to a rotational assembly and atherectomy tool. For example, such configuration may comprise a fluid bearing for the lateral transmission assembly of the atherectomy subsystem.

As reviewed above, the pulsatile intravascular lithotripsy subsystem further includes a distal balloon. Any convenient balloon may be employed. Suitable balloons include, but are not limited to, standard angioplasty balloons, such as compliant and non-compliant angioplasty balloons. In one embodiment, the balloon is a composite balloon that includes two distinct layers, which layers include a non-compliant layer and compliant layer. To describe the improvements of the current composite balloon structure over the prior art, the two layers of the composite balloon as individual units will be described. Non-compliant angioplasty balloons are typically used in percutaneous procedures because the set diameter of the balloon distributes its force equally to the surrounding vessel without bulging into the less stiff, healthy tissue surrounding a stenosis. When the non-compliant balloon material is pressurized, the balloon first fills, which yields a low pressure and high stretch state. When the non-compliant balloon reaches its nominal diameter, balloon pressure increases significantly for a correspondingly low stretch. When pressure is released in the balloon, the balloon remains at its nominal stretch because of the lack of elasticity in the balloon. This lack of elasticity is problematic for three reasons: (1) unless vacuum is generated, a de-pressurized balloon can remain filled, which can occlude blood flow, (2) it can lead to difficulty in removing the balloon catheter through the sheath after treatment, and (3) during pulsatile treatment, the balloon does not force fluid out during the low-pressure phase, which prevents the required stress relaxation in the surrounding tissues. Therefore, non-compliant balloons are useful at high pressures but have limitations at lower pressures. Compliant angioplasty balloons typically have a linear pressure-stretch curve. The use of these balloons is limited in percutaneous procedures because the balloon stretches non-uniformly around hardened segments of an artery, which may cause damage to the healthy, soft tissues surrounding the hardened diseased tissues. With a compliant balloon, there is typically a linear increase in balloon pressure. Compared to non-compliant balloons, compliant balloons have a “short” initial fill region, and therefore, when the pressure in the balloon is released, the balloon returns to its initial stretch state without requiring additional vacuum. This return to its initial state benefits certain pulsatile intravascular lithotripsy procedures because, upon return to the initial stretch state, blood flow is immediately restored, and the balloon can be more easily retracted through the sheath. Further, during pulsatile angioplasty, the compliance of the balloon serves as the impetus for forcing fluid out of the balloon, which is required to allow the surrounding tissue to relax with low stress during the low-pressure phases. Therefore, compliant balloons are helpful at lower pressures but are limited in their ability to treat at high pressures. Alone, non-compliant and compliant angioplasty balloons are not optimal for the various stages of pulsatile and standard percutaneous angioplasty. Together as a composite, though, they can meet important needs of both treatments. In one embodiment of the composite angioplasty balloon, a non-compliant balloon is covered with a compliant sleeve to achieve an “arrowed” pressure-stretch, e.g., as further described in pending PCT application serial no. PCT/US2020/055458; the disclosure of which is herein incorporated by reference. The compliant layer may be a rubber, silicone, polyurethane, or nitinol material or another material that can stretch up to 100-500% before failure, can withstand thousands of cycles before failure, and encounters minimal, if any, plastic deformation during expansion. During use, the exemplary composite balloon performs as follows. During the low-pressure phase, the compliant material dominates the response. The composite balloon follows the compliant material curve until the balloon stretch intersects with the non-compliant balloon stretch. At this intersection and higher pressures, the non-compliant material dominates the balloon response. Immediately upon pressure release, the balloon returns along the arrowed response to the initial, or zero, stretch state. This exemplary composite balloon has the low-pressure benefits of compliant angioplasty balloons and the high-pressure benefits of non-compliant angioplasty balloons. Other benefits include self-folding and deflating of the balloon, tear and pin-hole mitigation, increased oscillation frequency during pulsatile angioplasty, and increased ability of the balloon to withstand being pushed or otherwise forced in a direction, such as a longitudinal direction, while crossing a lesion. Further details regarding composite angioplasty balloons, etc. finding use in embodiments of the invention may be found in U.S. Pat. No. 11,464,949, United States Published Patent Application Publication No. 20200046949 as well as pending PCT Application Serial No. PCT/US2020/055458 and pending PCT Application Serial No. PCT/US2022/014785; the disclosures of which are herein incorporated by reference.

In other instances, the balloon may have external features that generate pulsatile stress concentrations in the surrounding materials. These features may be incorporated into the general balloon shape (i.e., when pressurized the balloon assumes the shape of the original mold) or via additional components (e.g., strips or cage) surrounding the balloon. In certain instances, the additional components traverse the length of the balloon (i.e., from proximal to distal end of the balloon). In this case, the stress concentration in the surrounding material is generated such that radial cracks are generated in the calcium structure. In other instances, the additional components traverse circumferentially around the balloon such that when the balloon lengthens during each pulse, longitudinal cracks form in the calcium structure. Further, the additional components may be distributed orthogonally such that both radial and longitudinal stress concentrations in the surrounding materials are generated. Thereby, both longitudinal and radial cracks can be generated for full calcium shattering.

The pulsatile intravascular lithotripsy subsystem may or may not be “sealed.” In some instances, the pulsatile intravascular lithotripsy subsystem is not sealed, such that a fluid, e.g., liquid, may be introduced into the liquid passageway(s) of the assembly at the time of use and/or gas may be removed from the subsystem (e.g., via de-bubbling). In yet other instances, the pulsatile intravascular lithotripsy subsystem is a sealed or closed assembly, such that the liquid passageways and distal balloon are pre-filled with a liquid prior to use, and the liquid is sealed in the assembly. In either instance, the liquid that is introduced into the lumen(s) and distal balloon may vary, where in some instances the liquid is saline. Where desired, the liquid may include a suitable contrast agent, where examples of contrast agents include, but are not limited to radiocontrast agents, such as but not limited to, iodine contrast agents, barium contrast agents, etc.

In some instances, the above embodiments may be configured such that a pulsatile intravascular lithotripsy procedure may be performed completely autonomously and/or remotely. In these cases, the catheter and distal balloon may be inserted into the patient manually or using a robotic catheterization system (as described in published application WO 2010/025338, the disclosure of which is herein incorporated by reference). With such a subsystem, equipment such as guidewires and the catheter and distal balloon can be advanced to the site of the lesion, and, once at the lesion, the balloon may be inflated or deflated. With the embodiments described above, the balloon may be pre-filled with fluid so that the user does not have to fill the balloon prior to pressurization. In other instances, the composite balloon embodiment may be used to ensure that the balloon deflates and wraps post-procedure so it may be easily removed. In such instances, the operator may be situated at a console to control the procedure including the pressure, frequency, and/or duty cycle. At the same time, the operator may be able see X-ray imaging at the same time to visualize the inflated balloon and procedural efficacy. In some instances, feedback, e.g., visual, audio, feel and the like, may be provided to the operator to indicate procedural characteristics such as volume and/or pressure change in the balloon, frequency, duty cycle, balloon expansion, balloon position and the like.

The pulsatile intravascular lithotripsy subsystem may be configured for single or one-time use, such that it is disposable. Prior to use, the pulsatile intravascular lithotripsy subsystem may be sterile, as desired.

Further details regarding aspects of, or components relevant to, systems of the present invention, including consoles, regulators, potential sources, handles, manifold assemblies, oscillators, pulse generators, balloon catheter assemblies, atherectomy subsystems, pulsatile intravascular lithotripsy subsystems, distal balloons, composite angioplasty balloons, etc., that may be incorporated into, or used in conjunction with, embodiments of the present invention are provided in U.S. Pat. No. 11,464,949; United States Published Patent Application Publication No. 20200046949; U.S. application Ser. No. 17/897,604; pending PCT Application Serial No. PCT/US2019/027139; pending PCT Application Serial No. PCT/US2020/055458; U.S. Application Ser. No. 63/274,832; U.S. application Ser. No. 17/827,169; pending PCT Application Serial No. PCT/US2022/014785; U.S. Application Ser. No. 63/238,381; pending PCT Application Serial No. PCT/US2022/040586; U.S. Application Ser. No. 63/346,703; pending PCT Application Serial No. PCT/US23/23533; U.S. Application Ser. No. 63/346,704; pending PCT Application Serial No. PCT/US23/22685; U.S. Application Ser. No. 63/444,414; U.S. Application Ser. No. 63/545,060; the disclosures of each of which are herein incorporated by reference.

### Certain Additional Features:

Certain embodiments of systems of the invention comprise an inner guidewire lumen present within a pulsatile intravascular lithotripsy catheter (such as, for example, where guidewire 257 is present within a guidewire lumen of catheter 254 of FIG. 2B). Such embodiments may be configured to allow low friction rotation even at high speeds, i.e., high frequency rotations, of the guidewire. Example techniques include employing a fluid bearing (using pressurized fluid such as saline, contrast, or other lubricant) such that, for example, fluid is present within such guidewire lumen. In embodiments, such fluid bearing may further work to dissipate heat generated by rotation of the guidewire within the guidewire lumen of the catheter. Other techniques comprise utilizing a self-lubricating material on an inner surface of the guidewire lumen of the catheter.

Where desired, a Y-hub or similar hub of the pulsatile intravascular lithotripsy catheter (e.g., connection 258 of FIG. 2B) may be connected to the atherectomy subsystem, e.g., ultimately connected to the atherectomy tool, in order to prevent windup or lashing of aspects of the system during atherectomy grinding, i.e., while the atherectomy tool is rotated via lateral transmission assembly comprising a guidewire (i.e., while atherectomy tool 269 is rotated by the rotation of guidewire 257).

Where desired, gas used to supply energy to the atherectomy subsystem and/or the pulsatile intravascular lithotripsy subsystem can be exhausted from, for example, a switch present within the system, back to the console. Such configuration may help to ensure that gas is exhausted into desired volumes and does not enter a sterile area, for example. Such routing of exhaust gas may be accomplished by including, for example, exhaust tubing within the handle and/or a tether and/or atherectomy subsystem and/or pulsatile intravascular lithotripsy subsystem.

In embodiments, the atherectomy subsystem utilizes rotational motion in order to rotate or orbit the atherectomy tool to thereby drill or grind a lesion. In some cases, such rotational motion is generated via compressed gas turning a turbine. Such turbine may be operably connected to a lateral transmission assembly comprising, e.g., a guidewire. In other cases, such rotational motion is generated via a motor and motor controller, e.g., an electromagnetic motor. When present, the motor may be located in the atherectomy subsystem, the handle, the console or another aspect of an embodiment of a system. The motor controller can be located in any of such locations but needn't be directly adjacent to the motor. When present, the motor may be connected directly to the lateral transmission assembly or a guidewire thereof. Alternatively, the motor can be connected to a lateral transmission assembly or a guidewire thereof that can be coupled to the atherectomy subsystem via a rotor connection or gear set. That is, the lateral transmission assembly may comprise gearing or other techniques for modulating or adjusting the rotation of the drive shaft such that the atherectomy tool rotates as desired.

Embodiments of systems of the invention may comprise one or more sensors configured to determine aspects of the rotation of the lateral transmission assembly and/or the atherectomy tool, e.g., aspects of the rotation of a guidewire operably connected to a griding burr. Sensors of interest include a light-based rotational sensor; a flow meter operably connected to the rotational assembly, e.g., a motor, e.g., operably connected to an outlet of a turbine of a rotational assembly; a current sensor on a rotational assembly, e.g., on a motor (i.e., generator) attached to an outlet of a rotational assembly; a Hall sensor, e.g., configured to sense a distance of position of an aspect of the atherectomy subsystem, e.g., the atherectomy tool or lateral transmission assembly; or the like.

Embodiments may be further configured to ascertain compliance measurements before, during or after treatment with the pulsatile intravascular lithotripsy subsystem. Such compliance measurements may be made based off of therapy with the atherectomy subsystem combined with compliance measurements from pulsatile intravascular lithotripsy therapy pre- and post-therapy.

### Methods

Systems of the invention find use in a variety of applications. In some instances, the systems find use in fracturing hardened materials embedded within an elastic conduit. For embodiments presented herein, the present disclosure describes applications related to treating atherosclerotic calcifications within an arterial conduit, such as a coronary or peripheral artery, for example. However, the present system and teachings are not solely limited to atherosclerotic calcifications nor arterial conduits and may be generally applied to other applications as determined by those skilled in the art. For example, this is especially true for circumstances that alter arterial compliance (vessel compliance, as described herein, of an artery) or for cases that involve medical interventions, such as the presence of a previous stent with subsequent blockage. The compliance of the vessel is altered by the intra-luminal placement of a previous stent. Data and feedback of vessel compliance curves can be used in connection with future therapies as well as for prediction techniques, such as machine learning techniques described herein.

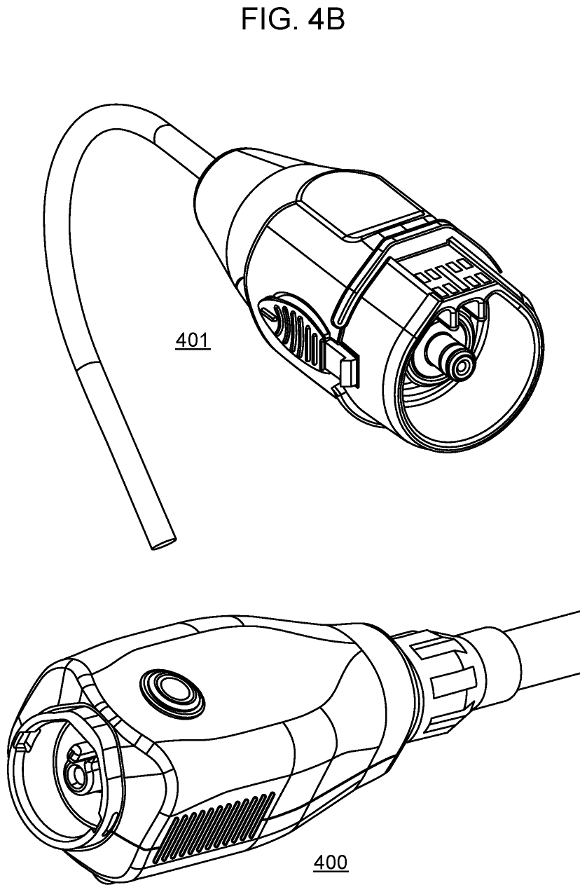

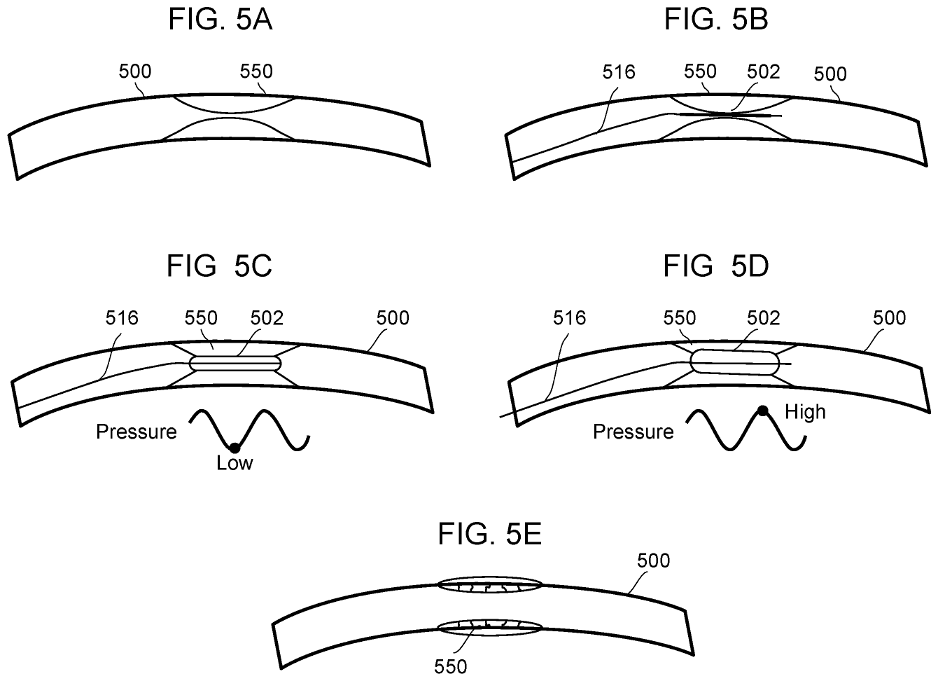

In some instances, the various embodiments of the systems described herein are employed in methods of pulsatile intravascular lithotripsy, e.g., a technique that uses pressure oscillations with a generalized waveform (in some embodiments, harmonic, or frequency-specific, pressure waveform oscillations) to effectively and safely fracture calcified lesions during angioplasty. Systems may find use in methods of pulsatile intravascular lithotripsy after such system is used to perform an atherectomy procedure, where such atherectomy procedure enables aspects of the pulsatile intravascular lithotripsy subsystem to access a lesion by drilling or grinding the lesion. The concept of DBA for treating arterial calcified plaque is illustrated in FIGS. 5A-E. In DBA, a catheter 516 with balloon 502 is deployed to the vessel 500 with calcification 550 (FIG. 5B), e.g., with the assistance of a guidewire using any convenient protocol, such as those known in the art.

In embodiments of the invention, an atherectomy subsystem of the system is employed first to provide or expand a pass through the occlusion 550. In such cases, an atherectomy tool may be utilized to provide or expand a pass through the occlusion 550. The atherectomy tool may, as described herein, comprise a griding burr, such that the atherectomy tool is rotated to grind aspects of lesion 550 such that balloon 502 can access vessel 500 via lesion 550. In embodiments of systems of the invention comprising combined tools, the atherectomy tool may be operably connected to, e.g., located distal to, balloon 502; for example, such elements may be connected via a guidewire.

Through the angioplasty balloon, the plaque is subjected to high-frequency pressure oscillations (FIGS. 5C-D). In the low-pressure phase of the oscillations (FIG. 5C), the balloon pressure is reduced to near the minimum pressure needed to achieve balloon inflation, typically 1-2 atm. In the high-pressure phase of the oscillations (FIG. 5D), the balloon is inflated to a peak pressure, which can be set by the operator, e.g., a physician, or automatically determined and set by the system. Typical peak inflation pressures may be from above the low-pressure range to the balloons' max rated pressure, which could be 25 atm or more. Pressure cycling of the balloon in this manner induces cyclical loading of the calcified plaque 550 below the calcified plaque's rupture stress yet in the plastic deformation zone. The heterogeneity of the calcified plaque interior is composed of many microfractures with sharp corners. Through the fracture mechanisms described herein, the cyclic loading described in FIGS. 5A-E causes cyclic stresses at these sharp corners near plaque microfractures and irregular surfaces. The cyclic stress initiates and grows these sharp corners, which expand the microfractures into larger macroscopic fractures. The growth of these microfractures leads to the more complete fracture of plaque at lower inflation pressures compared to static pressure. Higher frequency pressure cycles and higher-pressure differences between the cycles is expected to increase the effectiveness of this crack growth mechanism. By generating controlled high-frequency pressures cycles in an angioplasty balloon, DBA lowers the required balloon pressure for fracturing calcified plaque (e.g., in some instances between 1 to 50%, such as 20 to 30%, as compared to a suitable non-DBA control), improves stent deployment, improves and controls drug delivery for drug-coated balloons, stress-softens soft, lipid-core atheromatous plaques, expands calcified in-stent restenosis, and fracture calcifications on diseased cardiac valve leaflets and improves balloon-based expansion and deployment of prosthesis and devices. Further details regarding embodiments of DBA methods in which the systems described herein may be employed are provided in U.S. Pat. No. 11,464,949, United States Published Patent Application Publication No. 20200046949 as well as pending PCT Application Serial No. PCT/US2020/055458 and pending PCT Application Serial No. PCT/US2022/014785; the disclosures of which are herein incorporated by reference.

In some instances, systems of the invention, e.g., as described above, are employed in a manner sufficient to achieve a four-part pulsatile treatment plan, e.g., as illustrated in FIG. 6, which four-part pulsatile treatment plan provides for safe, controlled expansion of CP and surrounding healthy soft tissues. To treat these multi-compositional vessels, a four-part treatment algorithm may be employed, which four-part treatment algorithm includes the following steps: (1) soft-tissue low-stress expansion phase 680 via Mullins' Effect; (2) plastic deformation phase 670 in the calcified plaque until plaque fracture; (3) plaque fracture detection and immediate reduction in pressure phase 660 to reduce surrounding tissue stress; and (4) soft-tissue low-stress expansion phase 635 via Mullins' Effect to expand soft-tissue post-calcium fracture. An embodiment in which the force applied to the vessel over time by this four-part algorithm is illustrated in FIG. 6. Because of the significant attenuation across the catheter and the lack of pressure control in prior art approaches, the input pressure to the catheter is not successfully transmitted to the balloon (the system output). Therefore, tissue stress in prior art systems is not returned to a low state (i.e., there is no tissue relaxation period) during high frequency oscillation, which limits the effect of Mullins' stress cycling. Through attenuation minimization and pressure control, embodiments of the present system induce pressure oscillations in the distal balloon (the system output) that follows the pressure input from the proximal source with pressure oscillations between 0-50 ATM, frequencies between 0-25 Hz, and duty cycles between 60-80%. This advancement is important for two reasons: (1) it allows the tissue to relax during the depressurization cycle (following the Mullins' Effect) and (2) it allows sufficient oscillations to be applied to the vessel within the limited time during which an artery is occluded during treatment. Further details regarding methods in which embodiments of the invention may be used are found in U.S. Pat. No. 11,464,949, United States Published Patent Application Publication No. 20200046949 as well as pending PCT Application Serial No. PCT/US2020/055458 and pending PCT Application Serial No. PCT/US2022/014785; the disclosures of which are herein incorporated by reference.

In certain instances, embodiments of the present invention may be applied to assess vessel compliance. Blood vessels are naturally compliant, elastic structures. Their compliance is required to convert the pulsatile flow from the heart into steady flow in the capillaries. Over time, however, the aging and atherosclerotic process can diminish the compliance of vessels and reduce lumen area, creating flow mismatches and additional stress to the vascular system. Vessel compliance is especially diminished during and after the formation of intimal and medial calcified plaque in the vessel wall.

Improving vessel compliance is a prerequisite to a more definitive treatment of atherosclerosis. See Dattilo R, Himmelstein S I, Cuff R F. The COMPLIANCE 360 Trial: a randomized, prospective, multicenter, pilot study comparing acute and long-term results of orbital atherectomy to balloon angioplasty for calcified femoropopliteal disease. J Invasive Cardiol. 2014; 26 (8): 355-360. http://www.ncbi.nlm.nih.gov/pubmed/25091093. To maximize vessel wall compliance after calcium buildup, angioplasty or pulsatile intravascular lithotripsy therapy can be used to shatter intimal and medial calcium rings to expose the more elastic components of the tubular vessel and to release it from the mummifying calcium. As noted above, embodiments of the present invention may be used to apply pulsatile intravascular lithotripsy enabling both for cracking of calcium (i.e., cracking CP tissue as shown in FIGS. 5A-E) as well as to apply a final post-dilatation of the vessel, in each case after access is gained to a lesion by use of the atherectomy subsystem to perform an atherectomy procedure. In some cases, embodiments of the present invention may apply two applications, cracking CP tissue and applying a final post-dilation of the tissue, in a single treatment. That is, embodiments of the present invention can also be used to apply DBA first to apply pulses to a vessel to crack CP tissue and then to subsequently expand the vessel using, for example, a traditional, non-compliant balloon post-dilatation.

By vessel compliance, it is meant a measurable quantity defined by the following relationship:

$$C = \frac{Δ V}{Δ P}$$

where ΔV is the change in vessel volume for a given change in pressure ΔP. Because of tissue incompressibility, vessel volume can be converted to area by dividing by vessel length. Since the pressure-volume relationship in an artery is non-linear, compliance is often defined at a given pressure or volume.

Vessel compliance can be difficult to obtain because simultaneous in-vivo measurements of pressure change, ΔP, and vessel cross-sectional area or volume change, ΔA or ΔV, respectively, may be challenging. Systems according to the present invention find use in addressing this difficulty by accurately assessing vessel compliance in-vivo as described below.

As reviewed above, the proximal connector of the pulsatile intravascular lithotripsy subsystem may include a membrane positional sensor, such as a Hall sensor, which provides data regarding the spatial position of the membrane at any given time, as well as a pressure gauge for measuring pressure in the liquid passages and distal balloon of the balloon catheter assembly. In such instances, the systems may be employed to assess volume expansion of the balloon in real time, and/or vessel compliance at the site of the balloon.

As a consequence of measuring the diaphragm position, the change in volume in the distal balloon can be assessed in real time and the corresponding balloon pressure can be measured. That is, the system is configured to pressurize the distal balloon while simultaneously reading pressure and volume in the pulsatile intravascular lithotripsy subsystem. This volume-pressure relationship can provide a measure of vessel compliance, since, as described above, vessel compliance is the ratio of a change of vessel volume to a change in pressure. To enable this measurement, the balloon volume-pressure relationship can be measured when the distal balloon is uninhibited by a surrounding vessel. When located within a stiff vessel, the balloon requires a higher pressure for the equivalent balloon volume in its uninhibited, baseline state. Therefore, the balloon may be used to measure compliance of the vessel. With the ability to accurately record diaphragm position (a surrogate measurement for balloon volume) and balloon pressure, the compliance of the vessel can be measured easily in-vivo. This compliance measure can be used as a measure of a successful treatment with a lower compliance indicating adequate or therapeutic balloon expansion and a successful treatment. In some instances, the system is employed in methods analogous to those described in U.S. Published Application Publication No. 20150080747 (the disclosure of which is herein incorporated by reference), where membrane displacement is used as the measure of balloon volume.

Systems and methods for measuring vessel compliance according to the present invention may be configured to obtain pre-, during, and post-treatment pressure-volume measurements. Using the data obtained during these measurements, change in vessel compliance can be obtained to determine treatment efficacy. Change in vessel compliance may also be used to adjust therapeutic intensity and/or duration.

In addition, embodiments of the present invention may be used to generate pressure-volume (i.e., compliance curves) at various instances during treatment. A relative change in compliance pre- and post-treatment may be obtained. These changes may be compared amongst similar vessel segments to understand an appropriate level of compliance change.

In addition, methods according to the present invention may also comprise obtaining concomitant measures of intraarterial cross-section using other available measuring techniques such as ultrasound, cineangiography, computed tomography, intravascular ultrasound (IVUS), and/or optical coherence tomography (OCT). In some instances, systems according to the present invention may be configured to incorporate information obtained from such measurements, i.e., sensor fusion techniques. Volume and/or area measurements obtained through such visualization techniques may be combined with pressure and volume readings of embodiments of a pulsatile intravascular lithotripsy subsystem according to the present invention (i.e., measurements of changes or relative volume and/or pressure) to generate absolute compliance measurements of vessels with increased accuracy. Such absolute compliance measurements may then be used to compare treatments across vascular beds for optimizing treatments for both short- and long-term success. In addition, an absolute measure of the compliance curve of a vessel may be obtained and compared across treatment groups.

While systems and methods of measuring vessel compliance have been described in the context of systems according to the present invention, such systems and methods for measuring vessel compliance may be applied to other systems as well, such as systems configured to deliver static balloon angioplasty, pulsatile intravascular lithotripsy, cavitation-based intravascular lithotripsy, and/or externally-applied lithotripsy pulses, and/or atherectomy-based treatment alone or in conjunction with any such treatment. Additional details regarding methods in which systems of the invention may be employed include those described in U.S. Pat. No. 11,464,949, United States Published Patent Application Publication No. 20200046949 as well as pending PCT Application Serial No. PCT/US2020/055458 and pending PCT Application Serial No. PCT/US2022/014785; the disclosures of which are herein incorporated by reference.

As described, methods of embodiments of the present invention comprise using an embodiment a system of the invention, as such are described herein, for performing atherectomy, i.e., an atherectomy procedure. Methods of embodiments of the present invention comprise using an embodiment a system of the invention, as such are described herein, for performing pulsatile intravascular lithotripsy, i.e., a pulsatile intravascular lithotripsy procedure. Methods of embodiments of the present invention comprise using an embodiment a system of the invention, as such are described herein, for performing atherectomy and pulsatile intravascular lithotripsy, i.e., an atherectomy and pulsatile intravascular lithotripsy procedure. Embodiments of methods of the invention comprise performing atherectomy using the atherectomy subsystem of a system according to an embodiment of the present invention, and performing pulsatile intravascular lithotripsy using the pulsatile intravascular lithotripsy subsystem of a system according to an embodiment of the present invention.

Other embodiments of methods of the invention comprise deploying a system according to an embodiment of the invention so that an atherectomy tool of the system is adjacent to an occlusion of a diseased vessel, actuating the system such that the atherectomy tool creates a channel in the occlusion of the diseased vessel, guiding a balloon of the system through the channel, and actuating the system to impart pulsatile energy to the diseased vessel. In some cases, guiding the balloon comprises guiding the balloon to a region adjacent to the channel. In other cases, guiding the balloon comprises guiding the balloon to an interior region of the channel. In embodiments, actuating the system such that the atherectomy tool creates a channel comprises grinding a channel with the atherectomy tool. In other embodiments, actuating the system such that the atherectomy tool creates a channel comprises causing the atherectomy tool to rotate or orbit. In certain cases, creating a channel comprises creating a new channel, such as drilling a hole, or expanding an existing channel, in each case, such that a balloon of the pulsatile intravascular lithotripsy subsystem can be guided into the channel as desired to perform pulsatile intravascular lithotripsy therein.

Further details regarding methods in which embodiments of the invention may be used, including further details regarding pulsatile intravascular lithotripsy, dynamic balloon angioplasty (DBA), vessel compliance, etc. utilizing embodiments of systems of the present invention are provided in U.S. Pat. No. 11,464,949; United States Published Patent Application Publication No. 20200046949; U.S. application Ser. No. 17/897,604; pending PCT Application Serial No. PCT/US2019/027139; pending PCT Application Serial No. PCT/US2020/055458; U.S. Application Ser. No. 63/274,832; U.S. application Ser. No. 17/827,169; pending PCT Application Serial No. PCT/US2022/014785; U.S. Application Ser. No. 63/238,381; pending PCT Application Serial No. PCT/US2022/040586; U.S. Application Ser. No. 63/346,703; pending PCT Application Serial No. PCT/US23/23533; U.S. Application Ser. No. 63/346,704; pending PCT Application Serial No. PCT/US23/22685; U.S. Application Ser. No. 63/444,414; U.S. Application Ser. No. 63/545,060; the disclosures of each of which are herein incorporated by reference.

Notwithstanding the appended claims, the disclosure is also defined by the following clauses:

1. A system comprising:
a console;
a handle;
an atherectomy subsystem; and
a pulsatile intravascular lithotripsy subsystem.

2. A system comprising:
a console;
a handle, wherein the handle is configured to be interchangeably operably connectable to:
   an atherectomy subsystem, and
   a pulsatile intravascular lithotripsy subsystem.

3. The system according to clause 2, wherein the handle is operably connected to the atherectomy subsystem.

4. The system according to clause 2, wherein the handle is operably connected to the pulsatile intravascular lithotripsy subsystem.

5. The system according to any of the preceding clauses, wherein the atherectomy subsystem and the pulsatile intravascular lithotripsy subsystem each comprises an interface configured to operably connect to an interface of the handle.

6. The system according to clause 5, wherein the atherectomy subsystem interface and the pulsatile intravascular lithotripsy subsystem interface each comprises interlocks configured to interlock with the handle interface.

7. The system according to any of the preceding clauses, wherein the atherectomy subsystem comprises:
an atherectomy tool.

8. The system according to clause 7, wherein the atherectomy tool comprises:
a rotational atherectomy tool.

9. The system according to any of clauses 7 to 8, wherein the atherectomy tool comprises:
an orbital atherectomy tool.

10. The system according to any of clauses 7 to 9, wherein the atherectomy tool comprises one or more of:

a laser tool, an ultrasound tool, an electrohydraulic lithotripsy (EHL) cavitation emitter tool or a mechanotransduction tool.

11. The system according to any of clauses 7 to 10, wherein the atherectomy tool is configured to grind within a lesion.

12. The system according to any of clauses 7 to 11, wherein the atherectomy tool is present at a distal region of the system.

13. The system according to any of clauses 7 to 12, wherein the atherectomy tool is configured to cross a lesion.

14. The system according to any of clauses 7 to 13, wherein the atherectomy tool is configured to cross an occluded lesion.

15. The system according to any of clauses 7 to 14, wherein the atherectomy tool is configured to cross a chronic total occlusion (CTO).

16. The system according to any of clauses 7 to 15, wherein the atherectomy tool is configured to create a new channel within an occlusion.

17. The system according to any of clauses 7 to 16, wherein the atherectomy tool is configured to drill a hole in an occlusion.

18. The system according to any of any of clauses 7 to 17, wherein the atherectomy tool is a burr.

19. The system according to any of clause 18, wherein the burr is a grinding burr.

20. The system according to any of the preceding clauses, wherein the atherectomy subsystem comprises:
a rotational assembly configured to produce rotational energy.

21. The system according to clause 20, wherein the rotational assembly is operably connected to the console.

22. The system according to any of clauses 20 to 21, wherein the rotational assembly is configured to transduce energy transmitted from the console into rotational energy.

23. The system according to any of clauses 20 to 22, wherein the rotational assembly is operably connected to a potential source of the console.

24. The system according to any of any of clauses 20 to 23, wherein the rotational assembly is a motor.

25. The system according to any of the preceding clauses, wherein the system is configured to rotate an atherectomy tool.

26. The system according to any of any of the preceding clauses, wherein the system is configured to orbit an atherectomy tool.

27. The system according to any of any of the preceding clauses, wherein the system is configured to both rotate and orbit an atherectomy tool.

28. The system according to any of the preceding clauses, wherein the atherectomy subsystem comprises:

a lateral transmission assembly configured to propagate rotational energy from the rotational assembly to the atherectomy tool.

29. The system according to clause 28, wherein the lateral transmission assembly is positioned distal to the rotational assembly and proximal to the atherectomy tool.

30. The system according to any of clauses 28 to 29, wherein the lateral transmission assembly comprises a rotational drive shaft.

31. The system according to clause 30, wherein the rotational drive shaft is a flexible drive shaft.

32. The system according to any of clauses 28 to 31, wherein the lateral transmission assembly is configured to receive a guidewire.

33. The system according to clauses 28 to 32, wherein the lateral transmission assembly comprises a guidewire lumen.

34. The system according to any of clauses 28 to 33, wherein the lateral transmission assembly comprises a guidewire.

35. The system according to any of clauses 28 to 34, wherein the lateral transmission assembly comprises a fluid bearing.

36. The system according to clause 35, wherein the fluid bearing is configured to dissipate heat.

37. The system according to any of clauses 35 to 36, wherein the fluid bearing comprises fluid present within a guidewire lumen.

38. The system according to clause 34, wherein the guidewire is configured to impart stability to the lateral transmission assembly while propagating rotational energy from the rotational assembly to the atherectomy tool.

39. The system according to any of the preceding clauses, wherein the atherectomy subsystem comprises an atherectomy tool configured to utilize mechanotransduction of a guidewire to bore through a lesion.

40. The system according to any of the preceding clauses, wherein the atherectomy subsystem comprises:
an advancer configured to advance and retract an atherectomy tool in distal and proximal directions.

41. The system according to clause 40, wherein the advancer is configured to pulse the atherectomy tool into and out of a lesion.

42. The system according to any of the preceding clauses, wherein the atherectomy subsystem comprises:
a filter located distal to an atherectomy tool.

43. The system according to clause 42, wherein the filter is configured to protect vasculature from distal emboli.

44. The system according to any of the preceding clauses, wherein the atherectomy subsystem comprises:
a sensor.

45. The system according to clause 44, wherein the sensor is configured to sense one or more of: current, rotational position, speed, acceleration, temperature, linear position, torque, pressure or flow.

46. The system according to any of clauses 44 to 45, wherein the sensor is configured to sense current associated with the atherectomy subsystem interfacing with a lesion.

47. The system according to any of clauses 44 to 46, wherein the sensor is configured to sense a current response to the atherectomy subsystem penetrating a lesion.

48. The system according to any of the preceding clauses, wherein the lesion comprises calcified plaque.

49. The system according to any of the preceding clauses, wherein the pulsatile intravascular lithotripsy subsystem comprises a balloon catheter assembly.

50. The system according to clause 49, wherein the balloon catheter assembly comprises:
(a) a proximal connector operably connecting the balloon catheter assembly to the handle and configured to transduce a first pulse energy generated by a pulse generator to a second pulse energy;
(b) a distal balloon; and
(c) a catheter component comprising a fluidic passage operably positioned between the proximal connector and the distal balloon configured to propagate the second pulse energy from the proximal connector along the fluid passage to the distal balloon.

51. The system according to any of the preceding clauses, wherein the pulsatile intravascular lithotripsy subsystem comprises:
a proximal connector;
a distal balloon; and
a catheter,
wherein the distal balloon is operably connected to the catheter, and the catheter is operably connected to the proximal connector.

52. The system according to any of clauses 50 to 51, wherein the proximal connector is configured to operably connect to the handle.

53. The system according to any of clauses 50 to 52, wherein the proximal connector and a connector of the atherectomy subsystem each comprise an identical handle interface.

54. The system according to any of clauses 50 to 53, wherein the atherectomy subsystem and pulsatile intravascular lithotripsy subsystem are integrated together.

55. The system according to any of the preceding clauses, further comprising an integrated atherectomy and pulsatile intravascular lithotripsy subsystem comprising:
the atherectomy subsystem, and
the pulsatile intravascular lithotripsy subsystem.

56. The system according to any of the preceding clauses, wherein the system is a unified atherectomy and pulsatile intravascular lithotripsy system.

57. The system according to any of the preceding clauses, wherein the atherectomy subsystem and the pulsatile intravascular lithotripsy subsystem comprise a unitary tool.

58. The system according to any of the preceding clauses, wherein the atherectomy subsystem and the pulsatile intravascular lithotripsy subsystem comprise a common distal region.

59. The system according to any of the preceding clauses, wherein the pulsatile intravascular lithotripsy subsystem comprises a guidewire lumen, the atherectomy subsystem comprises a lateral transmission assembly comprising a guidewire, and the guidewire is present within the guidewire lumen.

60. The system according to clause 59, wherein an atherectomy tool is present on a distal region of the guidewire.

61. The system according to any of the preceding clauses, wherein the atherectomy subsystem comprises a guidewire, and wherein the pulsatile intravascular lithotripsy subsystem comprises the guidewire.

62. The system according to any of the preceding clauses, wherein the system is an over the wire (OTW) system.

63. The system according to clause 62, wherein a guidewire is present over a substantial portion of a length of the atherectomy subsystem and the pulsatile intravascular lithotripsy subsystem.

64. The system according to any of the preceding clauses, wherein the system is a rapid exchange (RX) system.

65. The system according to clause 64, wherein a guidewire is present over only a distal region of the atherectomy subsystem and the pulsatile intravascular lithotripsy subsystem.

66. The system according to any of the preceding clauses, wherein the system comprises a guidewire and a distal region of the guidewire is coated with a grinding material.

67. The system according to clause 66, wherein the grinding material coating comprises an atherectomy tool.

68. The system according to any of clauses 66 to 67, wherein the grinding material coating comprises a distal burr.

69. The system according to any of clauses 66 to 68, wherein the grinding material coating has a predetermined diameter selected based on treatment efficacy.

70. The system according to any of clauses 66 to 69, wherein the grinding material coating has a predetermined diameter selected based on a diameter of a distal balloon of the pulsatile intravascular lithotripsy subsystem.

71. The system according to clause 70, wherein the predetermined diameter is selected such that a distal balloon of the pulsatile intravascular lithotripsy subsystem can be inserted into a hole created by the grinding material.

72. The system according to any of the preceding clauses, wherein the pulsatile intravascular lithotripsy subsystem comprises a guidewire lumen.

73. The system according to any of clause 72, wherein a guidewire is present within the guidewire lumen of the pulsatile intravascular lithotripsy subsystem.

74. The system according to any of the preceding clauses, further comprising: a filter present on a guidewire, distal to the atherectomy tool.

75. The system according to clause 74, wherein the filter is configured to protect vasculature from distal emboli.

76. The system according to any of the preceding clauses, wherein the atherectomy subsystem and the pulsatile intravascular lithotripsy subsystem have separate distal regions.

77. The system according to any of the preceding clauses, wherein a catheter component of the pulsatile intravascular lithotripsy subsystem is separate from the atherectomy subsystem.

78. The system according to any of clauses 76 to 77, wherein the separate distal regions of the atherectomy subsystem and the pulsatile intravascular lithotripsy subsystem are configured to interface with a guidewire.

79. The system according to any of the preceding clauses, further comprising:
a console operably connected to a potential source operably connected to a handle assembly.

80. The system according to any of the preceding clauses, further comprising a controller configured to receive input from a source that is external to the system.

81. The system according to any of the preceding clauses, further comprising a controller configured to receive input from at least one of: results of an electrocardiogram, an intravascular pressure monitor, a blood volume monitor or an imaging system.

82. The system according to any of clauses 79 to 81, wherein the console is a first console, and the system comprises a plurality of operably connected consoles.

83. The system according to any of clauses 79 to 82, wherein the console comprises:
a pulse generator.

84. The system according to clause 83, wherein the pulse generator is configured to generate pneumatic pulse energy.

85. The system according to any of clauses 83 to 84, wherein the pulse generator is configured to produce a pressure pulse having an amplitude that is selected based on treatment efficacy.

86. The system according to any of clauses 83 to 85, wherein the pulse generator is configured to generate static pneumatic energy.

87. The system according to any of the preceding clauses, further comprising: a potential source.

88. The system according to any of clause 87, wherein the potential source is a voltage potential or an electromagnetic potential or a pressure potential.

89. The system according to any of clauses 79 to 88, wherein the console comprises a regulator configured to regulate a first energy from the potential source to a second energy.

90. The system according to clause 89, wherein the regulator is an active regulator configured to be controlled by an electrical signal.

91. The system according to any of clauses 89 to 90, wherein the regulator is a passive regulator configured to be preset to a specific output.

92. The system according to any of the preceding clauses, wherein the system comprises:
a controller configured to control a console.

93. The system according to clause 92, wherein the console comprises the controller.

94. The system according to any of clauses 92 to 93, wherein the controller is configured to:
receive input from at least one of the console, the handle, the atherectomy subsystem or the pulsatile intravascular lithotripsy subsystem, and
adjust a configuration of at least one of the console, the handle, the atherectomy subsystem or the pulsatile intravascular lithotripsy subsystem based at least in part on the input received.

95. The system according to any of clauses 92 to 94, wherein the controller is configured to receive input from a source external to the system.

96. The system according to any of clauses 92 to 95, wherein the controller is configured to receive input from at least one of: results of an electrocardiogram, an intravascular pressure monitor, a blood volume monitor or an imaging system.

97. The system according to any of clauses 92 to 96, wherein the console is a first console, and the system comprises a plurality of operably connected consoles.

98. The system according to clause 97, wherein the plurality of consoles are operably connected to a switch.

99. The system according to any of the preceding clauses, further comprising: a switch.

100. The system according to clause 99, wherein the switch is operably connected to an output of a potential source.

101. The system according to any of clauses 99 to 100, wherein the switch is configured to controllably transmit energy received from the potential source to one or more outputs.

102. The system according to any of clauses 99 to 101, wherein the switch is configured to output an oscillating magnitude of energy.

103. The system according to any of clauses 99 to 102, wherein the switch is configured to output oscillating output pressure.

104. The system according to any of clauses 99 to 103, wherein the switch is an oscillator.

105. The system according to any of clauses 99 to 104, wherein the switch is configured to output a static magnitude of energy.

106. The system according to any of clauses 99 to 105, wherein the switch is configured to output a static output pressure.

107. The system according to any of clauses 104 to 106, wherein the oscillator is configured so that the oscillation frequency of the oscillator is synchronized with the results of an electrocardiogram.

108. The system according to any of clauses 99 to 107, wherein the switch comprises a mechanical switch or an electrical switch.

109. The system according to any of clauses 99 to 108, wherein the switch comprises a solenoid.

110. The system according to any of the preceding clauses, further configured to detect system states.

111. The system of any of the preceding clauses, wherein the handle is operably connected to the console.

112. The system of any of the preceding clauses, wherein the handle is configured to be operably connected to each of the atherectomy subsystem and the pulsatile intravascular lithotripsy subsystem.

113. The system of any of the preceding clauses, wherein the handle is configured to be operably connected to (a) a first connector of the atherectomy subsystem and (b) a second connector of the pulsatile intravascular lithotripsy subsystem.

114. The system of any of the preceding clauses, wherein the handle is configured to be releasably connected to each of the atherectomy subsystem and the pulsatile intravascular lithotripsy subsystem.

115. The system of any of the preceding clauses, wherein the handle is operably connected to the atherectomy subsystem and the pulsatile intravascular lithotripsy subsystem.

116. The system of any of the preceding clauses, wherein the handle comprises an interlock configured to interlock with each of the atherectomy subsystem and the pulsatile intravascular subsystem.

117. The system of any of any of the preceding clauses, wherein the handle comprises:
a single unit housing a switch configured to transmit energy to a subsystem connected to the handle.

118. The system of any of the preceding clauses, further comprising:
a rotational assembly configured to produce rotational energy for the atherectomy subsystem.

119. The system of any of the preceding clauses, wherein the handle is configured to be held by an operator.

120. The system of any of the preceding clauses, wherein the handle is configured to be held by an operator during use.

121. The system of any of the preceding clauses, wherein the handle weighs between 0.5 lbs. and 2.5 lbs.

122. The system of any of the preceding clauses, wherein the handle has a circumference of between 1.5 in and 5.0 in.

123. The system of any of the preceding clauses, wherein the handle has a length of between 4.0 in and 8.0 in.

124. The system of any of the preceding clauses, wherein the handle comprises one or more tactile features.

125. The system of clause 124, wherein the tactile features comprise groves or indentations.

126. A system comprising:
a console;
a handle; and
an integrated atherectomy and pulsatile intravascular lithotripsy subsystem comprising:
   an atherectomy subsystem; and
   a pulsatile intravascular lithotripsy subsystem.

127. A console according to any of the preceding clauses.

128. A handle according to any of the preceding clauses.

129. An atherectomy subsystem of a system according to any of clauses 1 to 126.

130. A pulsatile intravascular lithotripsy subsystem of a system according to any of clauses 1 to 126.

131. A method of performing pulsatile intravascular lithotripsy using a system according to any of any of clauses 1 to 126.

132. A method of performing atherectomy using a system according to any of any of clauses 1 to 126.

133. A method of performing atherectomy and pulsatile intravascular lithotripsy using a system according to any of any of clauses 1 to 126.

134. A method comprising:
performing an atherectomy procedure using the atherectomy subsystem of a system according to any of any of clauses 1 to 126; and
performing pulsatile intravascular lithotripsy using the pulsatile intravascular lithotripsy subsystem of the system.

135. A method comprising:
introducing into luminal tissue a combined tool of an atherectomy subsystem and pulsatile intravascular subsystem of a system according to any of clauses 1 to 126;
performing an atherectomy procedure using the atherectomy subsystem; and
performing a pulsatile intravascular lithotripsy procedure using the pulsatile intravascular lithotripsy subsystem.

136. A method comprising:
introducing a guidewire into luminal tissue;
using the guidewire to introduce into luminal tissue an atherectomy tool of an atherectomy subsystem of a system according to any of clauses 1 to 126;
removing the atherectomy subsystem from the luminal tissue; and
using the guidewire to introduce into luminal tissue a distal balloon of a pulsatile intravascular lithotripsy subsystem of the system.

137. A method for treating a diseased vessel, the method comprising:
deploying a system according to any of any of clauses 1 to 126 so that an atherectomy tool of the system is adjacent to an occlusion of a diseased vessel;
actuating the system such that the atherectomy tool creates a channel in the occlusion of the diseased vessel;
guiding a balloon of the system through the channel; and
actuating the system to impart pulsatile energy to the diseased vessel.

138. The method of clause 137 wherein guiding the balloon comprises guiding the balloon to a region adjacent to the channel.

139. The method of any of clauses 137 to 138 wherein guiding the balloon comprises guiding the balloon to an interior region of the channel.

140. The method of any of clauses 137 to 139 wherein actuating the system such that the atherectomy tool creates a channel comprises grinding a channel with the atherectomy tool.

141. The method of any of clauses 137 to 140 wherein actuating the system such that the atherectomy tool creates a channel comprises causing the atherectomy tool to rotate or orbit.

142. A kit comprising one or more components of a system according to any of clauses 1 to 126.

143. The kit according to clause 142 further comprising packaging for the one or more components.

144. The kit according to any of clauses 142 to 143, wherein one or more components of the kit are re-usable.

145. The kit according to any of clauses 142 to 144, wherein one or more components of the kit are sterile.

It is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

In at least some of the described embodiments, one or more elements used in an embodiment can interchangeably be used in another embodiment unless such a replacement is not technically feasible. It will be appreciated by those skilled in the art that various other omissions, additions and modifications may be made to the methods and structures described herein without departing from the scope of the claimed subject matter. All such modifications and changes are intended to fall within the scope of the subject matter, as defined by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference, including, but not limited to, disclosing and describing the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

Although the invention is described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the disclosure hereof merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

While the systems, devices, methods and kits have or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

What is claimed is:

1. A system comprising:
a console; and
a handle, wherein the handle is configured to be interchangeably operably connectable to:
an atherectomy subsystem, and
a pulsatile intravascular lithotripsy subsystem.

2. The system according to claim 1, wherein the handle is operably connected to the atherectomy subsystem.

3. The system according to claim 1, wherein the handle is operably connected to the pulsatile intravascular lithotripsy subsystem.

4. The system according to any of the preceding claims, wherein the atherectomy subsystem and the pulsatile intravascular lithotripsy subsystem each comprises an interface configured to operably connect to an interface of the handle.

5. The system according to any of the preceding claims, wherein the atherectomy subsystem comprises:
an atherectomy tool that is a rotational atherectomy tool or an orbital atherectomy tool or a laser tool or an ultrasound tool or an electrohydraulic lithotripsy (EHL) cavitation emitter tool or a mechanotransduction tool.

6. The system according to any of the preceding claims, wherein the atherectomy subsystem comprises:
a rotational assembly configured to transduce energy transmitted from the console into rotational energy.

7. The system according to any of the preceding claims, wherein the atherectomy subsystem comprises:
a lateral transmission assembly configured to propagate rotational energy from the rotational assembly to an atherectomy tool.

8. The system according to any of the preceding claims, wherein the atherectomy subsystem comprises:
a sensor configured to sense one or more of: current, rotational position, speed, acceleration, temperature, linear position, torque, pressure or flow.

9. The system according to any of the preceding claims, wherein the pulsatile intravascular lithotripsy subsystem comprises:
a proximal connector configured to operably connect to the handle;
a distal balloon; and
a catheter,
wherein the distal balloon is operably connected to the catheter, and the catheter is operably connected to the proximal connector.

10. The system according to claim 9, wherein the proximal connector and a connector of the atherectomy subsystem each comprise an identical handle interface.

11. The system according to any of the preceding claims, further comprising an integrated atherectomy and pulsatile intravascular lithotripsy subsystem comprising:
the atherectomy subsystem, and
the pulsatile intravascular lithotripsy subsystem.

12. The system according to any of the preceding claims,
wherein the pulsatile intravascular lithotripsy subsystem comprises a guidewire lumen, the atherectomy subsystem comprises a lateral transmission assembly comprising a guidewire, and the guidewire is present within the guidewire lumen.

13. The system according to claim 12, wherein the atherectomy tool is present on a distal region of the guidewire.

14. A method for treating a diseased vessel, the method comprising:
deploying a system according to any of any of claims 1 to 13 so that an atherectomy tool of the system is adjacent to an occlusion of a diseased vessel;
actuating the system such that the atherectomy tool creates a channel in the occlusion of the diseased vessel;
guiding a distal balloon of the system through the channel; and
actuating the system to impart pulsatile energy to the diseased vessel.

15. A method comprising:
introducing a guidewire into luminal tissue;
using the guidewire to introduce into luminal tissue an atherectomy tool of an atherectomy subsystem of a system according to any of claims 1 to 13;
removing the atherectomy subsystem from the luminal tissue; and
using the guidewire to introduce into luminal tissue a distal balloon of a pulsatile intravascular lithotripsy subsystem of the system.
